SEQUENCE LISTING (9 sequences)

SEQ ID NO: 1 - protein, 301 aa, Homo sapiens
MFSHLPFDCVLLLLLLLLTRSSEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTLQRDFTAAFPRMLTTRGHGPAETQTLGSLPDINLTQISTLANELRDSRLANDLRDSGATIRIGIYIGAGICAGLALALIFGALIFKWYSHSKEKIQNLSLISLANLPPSGLANAVAEGIRSEENIYTIEENVYEVEEPNEYYCYVSSRQQPSQPLGCRFAMP

SEQ ID NO: 2 - protein, 621 aa, Pongo pygmaeus
MGEPQQVSALPPPPMQYIKEYTDENIQEGLAPKPPPPIKDSYMMFGNQFQCDDLIIRPLESQGIERLHPMQFDHKKELRKLNMSILINFLDLLDILIRSPGNIKREEKLEDLKLLFVHVHHLINEYRPHQARETLRVMMEVQKRQRLETAERFQKHLERVIEVIQNCLASLPDDLPHSEAGMRVKTEPMDADDSNNCTGQNEHQRENSGSSEVKYIAEVGQNAYLPCFYTPAAPGNLVPVCWGKGACPVFECGSVVLRTDERDVNHRTSSRYWLNGDFRKGDVSLTIENVTLADSGIYCCRIQIPGIMNDEKFNLKLVIKPAKVTPAPTLQRDFTAAFPRMLTTGGHGPAETQTPWSLRDINLTQIPTLDKELRDSGLANELRDSTIRIGIYIGAGISAGLALALIFGALIFKWYSHSKEKIQNLSLISLANLPPSGLANAVAEGIRSEENIYTIEENVYEVEEPNEYYCYVSSGMKWRSPMSITAMSAVGSNPHNLWVVALQCHRSNHLIFELGVVFFRNYELCQLTGFGGSVHCYGAEFSHFQKIMTHMGIELGPALNLNRHVIASVFKPTELLNPETVNHGCMAQSRLTVTSASWVQAILLPQPPEWLGLQACTTMPN

SEQ ID NO: 3 - protein, 281 aa, Mus musculus
MFSGLTLNCVLLLLQLLLARSLEDGYKVEVGKNAYLPCSYTLPTSGTLVPMCWGKGFCPWSQCTNELLRTDERNVTYQKSSRYQLKGDLNKGDVSLIIKNVTLDDHGTYCCRIQFPGLMNDKKLELKLDIKAAKVTPAQTAHGDSTTASPRTLTTERNGSETQTLVTLHNNNGTKISTWADEIKDSGETIRTAIHIGVGVSAGLTLALIIGVLILKWYSCKKKKLSSLSLITLANLPPGGLANAGAVRIRSEENIYTIEENVYEVENSNEYYCYVNSQQPS

SEQ ID NO: 4 - protein, 280 aa, Bos taurus
MFSHLLFDCVLLMLLLLTSSLKGAYVSQVGQNADLPCTYSPATTENLVPVCWGKGPCPVFECYSLVLRTDGRNVTYQTSSRYLLKRDLHKGDVTLTIKNVTLADSGTYCCRIQFPGLMNDRKSNLELIIKPAKVTPAWTPWRDITTAFPRMLTTKGPVSETRTLKTLHDKNQTEISTLATELQDMGATTRTGLYIGAGVFAGLALILISGGLILKWYSDRKEKIQNSSLITLANLSPSGLANTAAEGMHPVENIYIIEENIYEVEDPYECYCSVNSGHQS

SEQ ID NO: 5 - protein, 355 aa, Homo sapiens
MAFSGSQAPYLSPAVPFSGTIQGGLQDGLQITVNGTVLSSSGTRFAVNFQTGFSGNDIAFHFNPRFEDGGYVVCNTRQNGSWGPEERKTHMPFQKGMPFDLCFLVQSSDFKVMVNGILFVQYFHRVPFHRVDTISVNGSVQLSYISFQNPRTVPVQPAFSTVPFSQPVCFPPRPRGRRQKPPGVWPANPAPITQTVIHTVQSAPGQMFSTPAIPPMMYPHPAYPMPFITTILGGLYPSKSILLSGTVLPSAQRFHINLCSGNHIAFHLNPRFDENAVVRNTQIDNSWGSEERSLPRKMPFVRGQSFSVWILCEAHCLKVAVDGQHLFEYYHRLRNLPTINRLEVGGDIQLTHVQT

SEQ ID NO: 6 - RNA, 16 nt, Artificial Sequence
  Description of Artificial Sequence Synthetic oligonucleotide
caaaccaggguauucu

SEQ ID NO: 7 - DNA, 33 bp, Artificial Sequence
  Description of Artificial Sequence Synthetic oligonucleotide
agacactggtgaccctccataataacaatggaa

SEQ ID NO: 8 - DNA, 23 bp, Artificial Sequence
  Description of Artificial Sequence Synthetic oligonucleotide
cggagagaaatggttcagagaca

SEQ ID NO: 9 - DNA, 21 bp, Artificial Sequence
  Description of Artificial Sequence Synthetic oligonucleotide
ttcatcagcccatgtggaaat

(19) United States
(12) Patent Application Publication
(10) Pub. No.: US 2010/0061992 A1
(43) Pub. Date: Mar. 11, 2010

(54) THERAPEUTIC USES OF TIM-3 MODULATORS

(75) Inventors: David E. Anderson, Brookline, MA (US); Ana C. Anderson, Brookline, MA (US); Vijay K. Kuchroo, Newton, MA (US); David A. Hafler, Boston, MA (US)

Correspondence Address: DAVID S. RESNICK, NIXON PEABODY LLP, 100 SUMMER STREET, BOSTON, MA, 02110-2131, US

(73) Assignee: THE BRIGHAM AND WOMEN'S HOSPITAL, INC., Boston, MA (US)

(21) Appl. No.: 12/514,602

(22) PCT Filed: Nov. 15, 2007
(86) PCT No.: PCT/US07/24067

§ 371 Date: May 13, 2009

Related U.S. Application Data
(60) Provisional application No. 60/859,391, filed on Nov. 15, 2006.
(60) Provisional application No. 60/923,945, filed on Apr. 17, 2007.

(51) Int. Cl. A61K 39/395; A61K 38/17; A61K 48/00; A61P 37/00; A61K 39/00; A61P 35/00

(52) U.S. Cl. 424/139.1; 424/130.1; 514/12; 514/44 R; 424/184.1

(57) ABSTRACT

The invention provides novel methods of treating neurological disorders, including neurodegenerative disorders such as MS. The invention also provides novel methods of treating cancers, including glial tumors such as glioblastoma multiforme. The invention further provides vaccines and related uses.

### STATEMENT REGARDING FEDERALLY-SPONSORED RESEARCH OR DEVELOPMENT

The invention described herein was supported, in whole or in part, by the National Institute of Health Grant Nos P01NS038087 and NS045937. The United States government has certain rights in the invention.

### BACKGROUND OF THE INVENTION

Multiple sclerosis (“MS”) affects approximately 1 out of 1,600 people in the United States and is a common cause of persistent disability in young adults. MS involves repeated episodes of inflammation of central nervous system tissue in any area of the brain and spinal cord. The inflammation destroys the myelin sheath covering the nerve cells in the affected area. This leaves multiple areas of scar tissue (sclerosis) along the covering of the nerve cells. Sclerosis slows or blocks the transmission of nerve impulses in that area, resulting in the development of the symptoms of MS.

The location of the inflammation varies from person to person and from episode to episode causing a variety of neurological pathologies that vary between individuals. Neurological signs associated with MS encompass a wide array of symptoms including limb weakness, compromised motor and cognitive function, sensory impairment, bladder disorders, sexual dysfunction, fatigue, ataxia, deafness and dementia.

The majority of patients with MS follow a relapsing-remitting course in the early stages of the disease, characterized by increased severity of existing symptoms and the appearance of new symptoms, followed by variable periods of total or partial recovery. Such relapsing-remitting MS may be inactive for several years between attacks. However, most patients with relapsing-remitting MS ultimately enter a secondary chronic progressive phase, characterised by progressive disability and classified as secondary progressive MS.

The secondary progressive phase of MS is characterized by continuous demyelination and progressive neurodegeneration. It is hypothesized that chronic demyelination is mediated by macrophages/microglia, continuing to attack myelin. This continuous demyelination leads to more dystrophic neurons that over time can no longer be remyelinated, leading to progressive neurodegeneration.

There is presently no known cure for MS. Treatment is aimed at controlling symptoms and maintaining function to give the maximum quality of life.

Glioblastoma is the most common primary CNS malignant neoplasm in adults, and accounts for nearly 75% of the cases. Although there has been steady progress in their treatment due to improvements in neuro-imaging, microsurgery and radiation, glioblastomas remain incurable. The average life expectancy is less than one year from diagnosis, and the five-year survival rate following aggressive therapy including gross tumor resection is less than 10%. Glioblastomas cause death due to rapid, aggressive, and infiltrative growth in the brain. The infiltrative growth pattern is responsible for the un-resectable nature of these tumors. Glioblastomas are also relatively resistant to radiation and chemotherapy, and therefore post-treatment recurrence rates are high. In addition, the immune response to the neoplastic cells is mainly ineffective in completely eradicating residual neoplastic cells following resection and radiation therapy (Roth, 1999; Dix, 1999; Sablotzki, 2000).

The extent of activation of either the humoral or cell-mediated branch of the immune system can determine the effectiveness of a vaccine against a particular disease. Furthermore, the development of immunologic memory by inducing memory-cell formation can be important for an effective vaccine against a particular disease (see for example, Paul, Fundamental Immunology, 4th Edition, 1999). The effectiveness of a vaccine at preventing or ameliorating the symptoms of a particular disease can depend on the type and strength of immune response generated by the vaccine.

Immune responses to many different antigens (e.g., antigens derived from infectious organisms, autoantigens or tumor antigens), while detectable, are frequently of insufficient magnitude or type to afford protection against a disease process mediated by agents (e.g., infectious microorganisms or tumor cells) expressing those antigens. In such situations, it is often desirable to administer to an appropriate subject, together with the antigen, an adjuvant that serves to enhance the immune response to the antigen in the subject. There remains an urgent need to provide better vaccines which can elicit systemic, non-specific as well as antigen-specific immune responses that are safe, can be repeatedly administered, and which are effective to prevent and/or treat diseases amenable to treatment by elicitation of an immune response, such as infectious disease, allergy and cancer.

### SUMMARY OF THE INVENTION

The present invention broadly relates to reagents, compositions and methods for the treatment of disorders. In one aspect the invention provides adjuvants and methods to increase immune response to an antigen. Another aspect relates to the treatment of nervous system disorders having an inflammatory component. Another aspect relates to the treatment of tumors, and in particular tumors of the central nervous system. Another aspect relates to the improved formulation of vaccines and their uses to treat, prevent or reduce the likelihood of viral and bacterial infections.

Each of these aspects is based upon the discovery of a relationship between TIM-3 and immune or inflammatory pathways.

Particular aspects of the present invention are based on the discovery that the TIM-3/galectin-9 pathway regulates the inflammatory activity of CD11b⁺ microglia in the central nervous system.

Aspects of the present invention relate to a method of treating inflammatory disease of the CNS in a subject, comprising administering to the subject a therapeutically effective amount of an agent that decreases TIM-3 activity in the subject. In one embodiment, the disease or disorder is Multiple Sclerosis. In another embodiment, the agent decreases the TIM-3 activity in antigen presenting cells (APCs). In another embodiment, the APCs are dendritic cells (DCs). In another embodiment, the APCs are CD11b⁺ microglia cells. In another embodiment, the CD11b⁺ microglia cells are located in the central nervous system. In another embodiment, the therapeutically-effective amount is an amount which decreases the inflammatory activity of APCs. In another embodiment, the subject is afflicted with secondary progressive multiple sclerosis and is not afflicted with relapsing remitting multiple sclerosis. In another embodiment, the subject is a human. In another embodiment the method improves at least one symptom of multiple sclerosis in the subject.

In one aspect, there is provided a method of treating multiple sclerosis in a subject in need of such treatment, the method comprising a) assessing whether the subject is in the remitting/relapsing or the secondary, progressive stage of multiple sclerosis, and, b) if the subject is determined to be in the secondary, progressive stage of multiple sclerosis, administering to the subject a therapeutically effective amount of an agent that decreases TIM-3 activity in the subject.

In one embodiment, administration of the agent decreases the TIM-3 activity in antigen presenting cells (APCs).

In another embodiment, the APCs comprise i) CD11b⁺ microglia cells, ii) CD11b⁺ monocytes, iii) dendritic cells (DCs), iii) or each of these populations. In another embodiment, the CD11b⁺ microglia cells are located in the central nervous system.

In another embodiment, the therapeutically-effective amount is an amount which decreases the inflammatory activity of APCs.

In another embodiment, the subject is a human.

In another embodiment, the agent is an antibody or an antigen-binding fragment thereof. In another embodiment, the antibody or fragment thereof binds to TIM-3. In another embodiment, the antibody or fragment thereof binds to the extracellular domain of TIM-3. In another embodiment, the agent is an antibody or antibody fragment that binds to a polypeptide comprising amino acids 30-128 of SEQ ID NO: 1. In another embodiment, the agent reduces the binding of galectin-9 to TIM-3.

In another embodiment, the agent comprises a polypeptide comprising amino acids 30-128 of SEQ ID NO: 1; or an amino acid sequence that is at least 90% identical to amino acids 30-128 of SEQ ID NO: 1 and which binds to galectin-9, inhibits release of TNF-α in APCs or both.

In another embodiment, the polypeptide agent is pegylated.

In another embodiment, the polypeptide comprises a human serum albumin polypeptide or fragment thereof; or an Fc domain of an immunoglobulin.

In another embodiment, the agent decreases the expression level a TIM-3 polypeptide or a galectin-9 polypeptide in the subject.

In another embodiment, the agent is a double stranded RNA oligonucleotide.

In another embodiment, the agent inhibits binding of full-length TIM-3 to a galectin-9 polypeptide.

In another embodiment, the agent inhibits binding of a polypeptide comprising amino acids 30-128 of SEQ ID NO: 1 to galectin-9. In another embodiment, the galectin-9 polypeptide comprises the amino acid sequence set forth in SEQ ID NO:5.

In another embodiment, the agent comprises a carbohydrate. In another embodiment, the carbohydrate is lactose or β-galactoside.

In another embodiment, the agent comprises a glycosylated polypeptide.

In another embodiment, the agent comprises pectin or modified pectin.

In another aspect, provided is a method of enhancing an immune response, the method comprising administering a Toll-like Receptor (TLR) agonist and an agent that increases a TIM-3 activity to a subject in need of an enhanced immune response.

In one embodiment, the method further comprises administering an antigen to which an immune response is to be generated with the TLR agonist and the agent that increases a TIM-3 activity.

In another embodiment, the TIM-3 activity is increased in antigen presenting cells (APCs) in the central nervous system. In another embodiment, the APCs comprise i) CD11b⁺ microglia cells, ii) CD11b+ monocytes, iii) dendritic cells (DCs), iii) or each of these populations.

In another embodiment, the agent is an antibody, or antigen-binding fragment thereof, or a polypeptide.

In another embodiment, the agent is a TIM-3 ligand.

In another embodiment, the TIM-3 ligand comprises a galectin-9 polypeptide.

In another embodiment, the agent is a polypeptide comprising at least one of the two carbohydrate recognition domains (CRD) of galectin-9. In another embodiment, the polypeptide comprises two CRD domains of galectin-9.

In another embodiment, the ligand comprises an amino acid sequence which is at least 90% identical to the amino acid sequence set forth in SEQ ID NO:5 and retains the capacity to bind TIM-3.

In another embodiment, the subject has a tumor, and the method treats the tumor. In another embodiment, the tumor is a central nervous system tumor. In another embodiment, the tumor is a glial tumor selected from astrocytomas, oligodendrogliomas, ependymoma, mixed gliomas, oligoastrocytomas, gangliogliomas, and glioblastoma multiforme.

Another aspect provided includes a vaccine composition comprising an agent that increases TIM-3 activity and a TLR ligand. In one embodiment, the agent increases TIM-3 activity in an antigen presenting cell (APC). This aspect is based, in part, on the observation that TIM-3 activation enhances the secretion of TNF-a by monocytes treated with lipopolysacharide (LPS). Where TLR agonists are being examined or used for the treatment of chronic conditions and/or cancer, the addition of a TIM-3 activator can further stimulate the immune response mediated by the TLR agonist. This effect can occur with, or without concurrent administration of antigen.

In another embodiment, the vaccine composition further comprises an antigen to which an immune response is to be generated, wherein the antigen is not the agent that increases TIM-3 activity.

In another embodiment, the agent is an antibody, or antigen-binding fragment thereof, or a polypeptide. In another embodiment, the antibody or antibody fragment binds TIM-3 and increases TIM-3 signaling.

In another embodiment, the agent is a TIM-3 ligand.

In another embodiment, the TIM-3 ligand comprises a galectin-9 polypeptide.

In another embodiment, the agent is a polypeptide comprising at least one of the two carbohydrate recognition domains (CRD) of galectin-9. In another embodiment, the polypeptide comprises two CRD domains of galectin-9.

In another embodiment, the polypeptide comprises an amino acid sequence which is at least 90% identical to the amino acid sequence set forth in SEQ ID NO:5 and retains the capacity to bind TIM-3.

In another embodiment, the vaccine composition further comprises a pharmaceutically acceptable carrier, excipient or diluent.

In another embodiment, the vaccine comprising the TLR ligand and the agent that increases TIM-3 activity are formulated in the same composition. Alternatively, in another embodiment, the vaccine comprising the TLR ligand and the agent that increases TIM-3 activity are formulated in a separate composition.

In another aspect, provided is a method of vaccinating an animal against an antigen, the method comprising administering to the animal an agent that increases TIM-3 activity, and a TLR ligand.

In one embodiment, the method further comprises administering the antigen to the animal, wherein the antigen is not the agent that increases TIM-3 activity.

In another embodiment, the agent increases TIM-3 in APCs upon vaccination of the animal.

In another embodiment, the agent is an antibody, or antigen-binding fragment thereof, or a polypeptide. In another embodiment, the antibody or antibody fragment binds TIM-3 and agonizes TIM-3 signaling.

In another embodiment, the agent is a TIM-3 ligand.

In another embodiment, the TIM-3 ligand comprises a galectin-9 polypeptide. In another embodiment, the agent is a polypeptide comprising at least one of the two carbohydrate recognition domains (CRD) of galectin-9. In another embodiment, the polypeptide comprises two CRD domains of galectin-9.

In another embodiment, the polypeptide comprises an amino acid sequence which is at least 90% identical to the amino acid sequence set forth in SEQ ID NO:5.

In another embodiment, the animal is a mammal. In another embodiment, the mammal is a human.

The invention further provides agents for the manufacture of medicaments to treat any of the disorders described herein. Any methods disclosed herein for treating or preventing a disorder by administering an agent to a subject may be applied to the use of the agent in the manufacture of a medicament to treat that disorder. For example, in one specific embodiment, a galectin-9 polypeptide may be used in the manufacture of a medicament for the treatment of a central nervous system tumor.

### BRIEF DESCRIPTION OF THE DRAWINGS

FIG. 1. TIM-3 expression on dendritic cells. a) Single cell suspensions from collagenase digested spleens from wild type and TIM-3^{-/-} mice were stained with anti-CD11b, anti-CD11c and anti-TIM-3 or RatIgG1. TIM-3 (open histogram) and isotype control (shaded histogram) staining on gated populations is shown. b) real-time PCR analysis of TIM-3 expression in sorted cell populations: Th1 cells (48 hrs post activation with anti-CD3, anti-CD28), CD4⁺ T cells, macrophages (CD11b⁺CD11c⁻), dendritic cells (CD11c⁺), and TIM-3 Chinese hamster ovary (CHO) cell transfectants. The mean of two independent experiments is shown. c) ex vivo PBMCs from a healthy subject were stained with antibodies to identify CD11b⁺ monocytes and CD11c⁺CD11b⁻ myeloid DCs as well as antibody to human TIM-3 or relevant isotype control. Staining with isotype control antibody is shown in shaded histogram. Similar observations have been seen in 6 independent experiments.

FIG. 2. TIM-3 on APCs promotes Th1 differentiation. a) Naïve CD4⁺ T cells were sorted from wild type and TIM-3^{-/-} DO11.10 transgenic mice and cultured with OVA 323-339 in the presence of either wild type or TIM-3^{-/-} APCs as indicated. Intracytoplasmic staining of CD4⁺ T cells for IFN-γ, IL-4 and IL-10 after one round of stimulation is shown. Similar results were obtained in 3 independent experiments. b) IFN-γ/IL-10 and IFN-γ/IL-4 ratio of wild type and TIM-3^{-/-} DO11.10 T cells cultured with wild type APCs (closed bars) and TIM-3^{-/-} APCs (open bars).

FIG. 3. TIM-3 function in dendritic cells. a) ex vivo splenic DCs from either wild type or TIM-3^{-/-} mice were isolated and cultured (3×10⁵/well) with galectin-9 (2 μg/ml), LPS (1 ng/ml), LPS⁺ galectin-9 or medium. Culture supernatant was collected after 18 hours and TNF-α, IL-6, IL-10 and IFN-γ production were measured by cytometric bead array. Similar results were obtained in three independent experiments. b) agonistic TIM-3 antibody activates TNF-α and NFkB in a dendritic cell line. D2SC1 cells were cultured (2.5×10⁵/well) with 10 μg/ml agonistic anti-TIM-3 antibody, Mouse IgG1 isotype control or LPS (1 ng/ml) c) Stimulation of ex vivo human monocytes with galectin-9 induced TNF-α in 10 independent experiments. Addition of anti-TIM-3 antibody alone had no effect on cytokine secretion. Error bars represent standard deviation in cytokine secretion from a representative experiment.

FIG. 4. TIM-3 expression in human microglia in white but not grey matter regions of the CNS. a) Tissue sections from white and grey matter regions of human non-inflamed CNS tissue were stained with TIM-3-specific monoclonal antibody. b) Dual immunofluorescence of non-inflamed CNS white matter tissue using monoclonal antibodies against CD11b and TIM-3. c) Quantitative RT-PCR analysis of TIM-3 mRNA levels in microglia isolated using LCM from white and grey matter regions of CNS tissue. Error bars represent standard deviation in TIM-3 mRNA levels among 5 experiments. Grey matter microglia express significantly lower levels of TIM-3 (p=0.02) based on a two-tailed t test. d) Comparative immunohistochemical staining of white and grey matter regions of human non-inflamed CNS tissue using HLA DR-specific monoclonal antibody. The more diffuse staining observed in grey matter is frequently observed with monoclonal antibody staining of grey matter microglia.

FIG. 5. TIM-3 expression in microglia differs depending on the nature of CNS inflammation. a) Microglia were isolated from non-inflamed (control) human CNS tissue (n=2), normal appearing white matter (NAWM) regions of MS tissue (n=2), the center (n=4) or border (n=3) regions of active MS plaques, or from glioblastoma multiforme (GBM) tumor specimens (n=3) and levels of TIM-3 were determine using quantitative RT-PCR. Error bars represent SEM. b) Microglia were isolated by FACS from 2 viable ex vivo preparations of MS plaque tissue and 2 GBM specimens and TIM-3 mRNA levels were determined by RT-PCR. Error bars represent SEM. c) Astrocytes were isolated by LCM from non-inflamed white matter (n=2) and from MS plaques (n=5) from two different brain specimens. Levels of the TIM-3 ligand galectin-9 were determined by RT-PCR. Error bars represent SEM.

FIG. 6. Analysis of TIM-3 on murine monocytes/macrophages and microglia. a) Splenocytes from immunized SJL were stained with monoclonal antibodies against CD11b, CD11c and TIM-3 (solid line) or Rat IgG1 isotype control (filled histogram). TIM-3 expression on splenic macrophages (CD11b⁺CD11c⁻) is shown. b) CNS mononuclear cells from a mouse with EAE were stained with monoclonal antibodies against CD11b, CD45 and TIM-3 or Rat IgG1 isotype control. TIM-3 expression on CNS microglia)(CD45^{lo}) and infiltrating macrophages (CD45^{hi}) during the course of EAE is illustrated relative to isotype control staining in shaded histogram. c) Mice were immunized for EAE and sacrificed at the indicated stages of disease. Each bar represents the mean of 2-3 individual mice.

FIG. 7. In vivo affect of agonistic anti-TIM-3 antibody. a) SJL mice were immunized with 100 μg of myelin proteolipid protein (PLP) 139-151 emulsified in incomplete Freund's adjuvant (IFA), IFA containing 100 μg Mouse IgG1, IFA containing 100 μg of anti-TIM-3 (5D12), or complete Freund's adjuvant (CFA) supplemented with 4 mg/ml Mycobacterium tuberculosis. Immunized mice (n=4/group) were monitored for the development of EAE. The mean clinical disease score in each group is shown. Results for two independent experiments are represented. b) Linear regression curves for anti-TIM-3 and mouse Ig groups are shown for the experiments represented in a). The slopes are significantly different between these groups for experiments one and two (p=0.0002 and p=0.0003, respectively). The 95% confidence intervals for each curve are represented with dashed lines.

FIG. 8 depicts an amino acid sequence alignment of the IgV domain of TIM-3 homologs from human TIM-3 (amino acid residues 22-131 of SEQ ID NO:1), Pongo pygmaeus (amino acid residues 211-321 of SEQ ID NO:2), Mus musculus (amino acid residues 22-132 of SEQ ID NO:3), and Bos taurus (amino acid residues 21-131 of SEQ ID NO:4).

FIG. 9 shows the effect of the stimulation of human monocytes with LPS (a TLR4 ligand/agonist) in the absence and presence of increasing amounts of galectin-9 on TNF-α secretion. Synergy between TIM-3 agonist and TLR agonist is demonstrated.

### DETAILED DESCRIPTION OF THE INVENTION

### I. Overview

CD4⁺ T helper cell subsets influence a wide array of human inflammatory diseases, and considerable effort has been devoted to elucidating the molecules and pathways that may regulate them. TIM-3 was originally identified as a molecule expressed specifically on terminally differentiated IFN-γ-secreting CD4⁺ Th1 cells that limits their function. TIM-3 is selectively expressed on fully differentiated Th1 but not Th2 cells (Monney, Nature. 2002 415 (6871):536-41), and has been shown in mice to regulate both the function of Th1 cells and the ability to induce tolerance (Sabatos, Nat. Immunol. 2003 November; 4 (11):1102-10; Sanchez-Fueyo, Nat. Immunol. 2003 November; 4 (11):1093-101). Galectin-9 is a ligand for TIM-3 and is disclosed in US Publication No. 2005/0191721, which is incorporated by reference. Galectin-9 is a member of the galectin family, is ubiquitously expressed on a variety of cell types and binds β-galactoside.

The galectin-9:TIM-3 interaction negatively regulates Th1 immunity by specifically inducing cell death in effector Th1 cells. In humans, Applicants recently demonstrated that potentially pathogenic Th1 T cell clones isolated from the cerebral spinal fluid of patients with multiple sclerosis (MS) express lower levels of TIM-3 than do those obtained from control subjects, consistent with a loss of T cell tolerance in the CNS. Thus, TIM-3 may critically regulate Th1 cells and maintenance of tolerance in the context of self-reactive T cells in human autoimmune disease.

Antigen presenting cells, including dendritic cells and macrophages, play a critical role in dictating the outcome of immune responses and are able to stimulate or suppress T cell activation depending on the manner in which they are stimulated. Microglia are the antigen presenting cells of the CNS, and are capable of activating infiltrating T cells. Indeed, microglial activation is associated with a variety of neurodegenerative and inflammatory diseases of the CNS.

Applicants have discovered that, in the naïve state, TIM-3 is constitutively expressed at high levels on cells of the innate immune system in both mouse and man, and that TIM-3 expression on these cells paradoxically promotes Th1 differentiation. Moreover, demonstrated herein is synergy between TIM-3 and TLR (Toll-like receptors) signaling. The significance of these results is examined in the context of two distinct CNS diseases. Significantly higher TIM-3 expression was found in APCs, specifically CD11b⁺ microglia, isolated from active MS lesions, relative to those isolated from resected glioblastoma tissue. In a murine model of multiple sclerosis, disease was exacerbated in the presence of agonistic antibody directed against TIM-3. Collectively, this disclosure demonstrates that in the innate immune system the presence of TIM-3 initially augments generation of Th1 immunity but can later terminate adaptive immunity, when its expression predominates on differentiated Th1 cells. These findings have relevance for a wide array of peripheral and organ-specific inflammatory human diseases as disclosed herein.

One aspect of the invention generally provides novel methods and agents for the treatment of nervous system disorders, particularly those disorders involving or characterized by inflammation. Preferred nervous system disorders include multiple sclerosis and glial tumors. The methods of the invention modulate TIM-3 activity in a subject to treat the disorders. The methods of the invention for decreasing TIM-3 activity can be useful for subjects who have a nervous system disorder such as a neurodegenerative disorder, while methods of increasing TIM-3 activity in a subject can be useful for subjects who have a glial tumor or other nervous system tumors.

Aspects of the invention are based in part on the unexpected finding that TIM-3 is present on APCs, including dendritic cells, human monocytes, and CD11b⁺ microglia, and that stimulation by a TIM-3 ligand such as galectin-9 results in TFNα secretion. Additionally, TIM-3 expression is increased in the active border regions of MS lesions, while TIM-3 expression is decreased in tissue obtained from a CNS tumor, glioblastoma multiforme.

One further aspect of the invention is based in part on the unexpected finding that TIM-3 expression is significantly lower in microglia isolated from glioblastoma multiforme brain tumor tissue compared to control tissue. The invention provides a method of treating a tumor in a subject, the method comprising administering to a subject a therapeutically effective amount of an agent that increases TIM-3 activity in the subject. In one embodiment the tumor is a central nervous system tumor. Central nervous system tumors include, but are not limited to meningiomas, pituitary adenomas, neuromas, and gliomas such as astrocytomas, oligodendrogliomas, ependymoma, mixed gliomas, oligoastrocytomas, gangliogliomas, and glioblastoma multiforme. In one embodiment, the tumor is a glial tumor.

One further aspect of the invention is based in part on the unexpected finding that TIM-3 activation in antigen presenting cells, or APCs, induces TNF-α secretion. Accordingly, the invention provides vaccine compositions comprising agents that increases TIM-3 activity as an adjuvant. These can be useful for viral, bacterial or cancer vaccines.

### II. Definitions

For convenience, certain terms employed in the specification, examples, and appended claims, are collected here. Unless defined otherwise, all technical and scientific terms used herein have the same meaning as commonly understood by one of ordinary skill in the art to which this invention belongs.

The articles “a” and “an” are used herein to refer to one or to more than one (i.e., to at least one) of the grammatical object of the article. By way of example, “an element” means one element or more than one element.

The term “antigen” includes all substances that can be recognized by the adaptive immune system. This includes, for example, viruses, bacteria, tumor-specific antigens, toxoids, polysaccharide conjugates, RNA, and DNA.

The term “including” is used herein to mean, and is used interchangeably with, the phrase “including but not limited” to.

The term “or” is used herein to mean, and is used interchangeably with, the term “and/or,” unless context clearly indicates otherwise.

The term “such as” is used herein to mean, and is used interchangeably, with the phrase “such as but not limited to”.

The term “nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides.

The term “preventing” is art-recognized, and when used in relation to a condition, such as a local recurrence (e.g., pain), a disease such as cancer, a syndrome complex such as heart failure or any other medical condition, is well understood in the art, and includes administering, prior to onset of the condition, a composition that reduces the frequency of, reduces the severity of, or delays the onset of symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount. Prevention of an infection includes, for example, reducing the number of diagnoses of the infection in a treated population versus an untreated control population, and/or delaying the onset of symptoms of the infection in a treated population versus an untreated control population. Prevention of pain includes, for example, reducing the frequency of, reducing the severity of, or alternatively delaying, pain sensations experienced by subjects in a treated population versus an untreated control population.

The term “effective amount” as used herein is defined as an amount effective, at dosages and for periods of time necessary to achieve the desired result. The effective amount of a compound of the invention may vary according to factors such as the disease state, age, sex, and weight of the animal. Dosage regimens may be adjusted to provide the optimum therapeutic response. For example, several divided doses may be administered daily or the dose may be proportionally reduced as indicated by the exigencies of the therapeutic situation.

A “subject” as used herein refers to any vertebrate animal, preferably a mammal, and more preferably a human. Examples of subjects include humans, non-human primates, rodents, guinea pigs, rabbits, sheep, pigs, goats, cows, horses, dogs, cats, birds, and fish.

A “variant” of a polypeptide of interest, as used herein, refers to an amino acid sequence that is altered by one or more amino acids. The variant may have “conservative” changes, wherein a substituted amino acid has similar structural or chemical properties, (e.g., replacement of leucine with isoleucine). More rarely, a variant may have “nonconservative” changes (e.g., replacement of a glycine with a tryptophan). Similar minor variations may also include amino acid deletions or insertions, or both. Guidance in determining which amino acid residues may be substituted, inserted, or deleted without abolishing biological or immunological activity may be found using computer programs well known in the art, for example, DNA-STAR software.

A “variant” as the term is used herein will retain at least one biological activity of the parent polypeptide. As used in this context, a “biological activity” excludes merely raising an immune response that generates antibodies to the parent or variant polypeptide.

As used herein, the term “inflammatory disorder of the central nervous sytem” refers to a disease or disorder of the CNS, the pathology of which involves or is characterized by an inflammatory component. The hallmarks of inflammation and activation of inflammatory pathways are recognized by those of skill in the art.

The term “adjuvant” refers to a substance that enhances, augments or potentiates the host's immune response to a vaccine antigen.

As used herein, the term “enhancing an immune response” refers to the improvement of an immune response upon administration of an agent or treatment, relative to the immune response in the absence of such agent or treatment. The skilled artisan can readily measure an immune response, and an “enhanced” immune response will be increased in a statistically significant amount, whether measured, e.g., by generation of antigen-specific antibodies, cell killing, or another measure of immune response. To avoid doubt, an “enhanced” response will generally be at least 10% greater than the response observed in the absence of the agent.

As used herein, “vaccine” means an organism or material that contains an antigen in an innocuous form. The vaccine is designed to trigger an immunoprotective response. The vaccine may be recombinant or non-recombinant. When inoculated into a non-immune host, the vaccine will provoke active immunity to the organism or material, but will not cause disease. Vaccines may take the form, for example, of a toxoid, which is defined as a toxin that has been detoxified but that still retains its major immunogenic determinants; or a killed organism, such as typhoid, cholera and poliomyelitis; or attenuated organisms, that are the live, but non-virulent, forms of pathogens, or it may be antigen encoded by such organism, or it may be a live tumor cell or an antigen present on a tumor cell.

As used herein, the term “Toll-like receptor agonist” or “TLR agonist” refers to an agent that activates a signalling activity of a Toll-like receptor.

It should be noted that where ranges are described herein, the ranges include and describe all integer values therebetween, as well as all sub-ranges, as if they were specifically recited herein. Thus, for example, the range 0 to 50% includes, not only 1%, 2%, 3%, 4% . . . 50%, but also as non-limiting examples, 0-40%, 0-30%, 5-45%, 5-40%, 5-10%, 10-30%, and 40-45%.

### III. TIM-3 and Galectin-9 Sequences

### A. Reference Sequences

In certain aspects, the present disclosure makes available isolated and/or purified forms of TIM-3 polypeptides and fragments thereof, which are isolated from, or are otherwise substantially free of, other proteins which might normally be associated with the protein or a particular complex including the protein.

In certain embodiments, a TIM-3 polypeptide is a polypeptide having an amino acid sequence that is at least 90%, at least 95%, at least 97%, at least 99% or 100% identical to SEQ ID NO: 1, which retains one or more signaling activities of the TIM-3 polypeptide of SEQ ID NO: 1. The term TIM-3 polypeptide also encompasses portions of such a polypeptide that retain one or more signaling activities of the polypeptide of SEQ ID NO: 1, as well as conservative substitution variants of TIM-3 polypeptide that retain one or more signaling activities of the polypeptide of SEQ ID NO: 1. At a minimum, a TIM-3 polypeptide can mediate the activation of CD11b⁺ microglia or bind galectin-9 or both. In one embodiment, the portion comprises the IgV domain of TIM-3. In some embodiments the TIM-3 polypeptide comprises amino acids 22-131 of SEQ ID: 1. In some embodiments, the soluble TIM-3 polypeptides contain one or more conservative amino acid substitutions relative to SEQ ID NO: 1.

In certain embodiments, a TIM-3 polypeptide is a polypeptide that is at least 90%, at least 95%, at least 97%, at least 99% or 100% identical to amino acids 22-131 of SEQ ID NO: 1. The amino acid identity between two polypeptides can be determined by first aligning the two polypeptide sequences using an alignment algorithm, such as one based on the PAM250 matrix.

In one embodiment of the methods described herein, the agent which increases TIM-3 activity is a TIM-3 ligand. As used herein, the term “TIM-3 ligand” refers to a molecule that binds an extracellular domain of TIM-3 and activates or inhibits one or more signaling activities of TIM-3. An example of such a ligand is galectin-9. In alternative embodiments, a TIM-3 ligand can compete for binding of a galectin-9 polypeptide. The polypeptide sequence of human galectin-9 may be found as Genbank Accession No. NP_{—}033665 and is also shown as SEQ ID NO:5. Accordingly, in some embodiments said agent comprises a galectin-9 polypeptide. Galectin-9 is known to bind TIM-3 via carbohydrates present or the TIM-3 IgV domain. The interaction involves the galectin-9 carbohydrate-recognition domain. In another embodiment, said agent comprises a polypeptide comprising at least one of the two carbohydrate recognition domains (CRD) of galectin-9 i.e. at least the N-terminal or the C-terminal, or both.

As used herein, the term “galectin-9 polypeptide” refers to a polypeptide having an amino acid sequence that is at least 90%, at least 95%, at least 97%, at least 99% or 100% identical to the polypeptide of SEQ ID NO: 5 and retains the ability to bind a TIM-3 polypeptide as that term is defined herein. The term “galectin-9 polypeptide” also encompasses fragments or conservative substitution variants of a galectin-9 polypeptide that retain the ability to bind a TIM-3 polypeptide. The term specifically encompasses polypeptides comprising one or both carbohydrate recognition domains of the galectin-9 polypeptide of SEQ ID NO: 5 and conservative substitution variants thereof that retain the ability to bind TIM-3 polypeptide. The crystal structures of both TIM-3 and galectin-9 (and particularly the CRDs) of galectin-9 are known. This knowledge provides guidance regarding the structures of galectin that are critical for TIM-3 binding. In a specific embodiment, the polypeptide comprises an amino acid sequence which is at least 80%, 90% or 95% identical to the amino acid sequence of at least one CRD of human galectin-9.

### B. Polypeptides Having Amino Acid Identity to Reference Sequences

The invention provides methods using polypeptides sharing a specified degree of sequence identity or similarity to a polypeptide. To determine the percent identity of two amino acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, at least 30%, at least 40%, at least 50%, at least 60%, at least 70%, at least 80%, at least 90%, at least 95% or more of the length of a reference sequence is aligned for comparison purposes. The amino acid residues at corresponding amino acid positions are then compared. When a position in the first sequence is occupied by the same amino acid residue as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences.

The comparison of sequences and determination of percent identity and similarity between two sequences can be accomplished using a mathematical algorithm. (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991).

In one embodiment, the percent identity between two amino acid sequences is determined using the Needleman and Wunsch (J Mol. Biol. (48):444-453 (1970)) algorithm which has been incorporated into the GAP program in the GCG software package (available at http://www.gcg.com). In a specific embodiment, the following parameters are used in the GAP program: either a Blossom 62 matrix or a PAM250 matrix, and a gap weight of 16, 14, 12, 10, 8, 6, or 4 and a length weight of 1, 2, 3, 4, 5, or 6. In yet another embodiment, the percent identity between two nucleotide sequences is determined using the GAP program in the GCG software package (Devereux, J., et al., Nucleic Acids Res. 12 (1):387 (1984)) (available at http://www.gcg.com). Exemplary parameters include using a NWSgapdna.CMP matrix and a gap weight of 40, 50, 60, 70, or 80 and a length weight of 1, 2, 3, 4, 5, or 6.

In another embodiment, the percent identity between two amino acid sequences is determined using the algorithm of E. Myers and W. Miller (CABIOS, 4:11-17 (1989)) which has been incorporated into the ALIGN program (version 2.0), using a PAM120 weight residue table, a gap length penalty of 12 and a gap penalty of 4.

Another embodiment for determining the best overall alignment between two amino acid sequences can be determined using the FASTDB computer program based on the algorithm of Brutlag et al. (Comp. App. Biosci., 6:237-245 (1990)). In a sequence alignment the query and subject sequences are both amino acid sequences. The result of said global sequence alignment is presented in terms of percent identity. In one embodiment, amino acid sequence identity is performed using the FASTDB computer program based on the algorithm of Brutlag et al. (Comp. App. Biosci., 6:237-245 (1990)). In a specific embodiment, parameters employed to calculate percent identity and similarity of an amino acid alignment comprise: Matrix=PAM 150, k-tuple=2, Mismatch Penalty=1, Joining Penalty=20, Randomization Group Length=0, Cutoff Score=1, Gap Penalty=5 and Gap Size Penalty=0.05.

Some aspects of the invention provide polypeptides, or provide therapeutic methods for employing those polypeptides, wherein said polypeptides are defined, at least in part, to a reference sequence. Accordingly, such polypeptides may have a certain percentage of amino acid residues which are not identical to a reference sequence. In one preferred embodiment, the non-identical residues have similar chemical properties to the residues to which they are not identical. Groups that have similar properties include the following amino acids: E, D, N, Q; H, K, R; Y, F and W; I, L, V, M, C, A; and S, T, C, P, A.

In another embodiment, the residues which are not identical are those which are not evolutionarily conserved between the reference sequence and an orthologous sequence in at least one evolutionarily related species, such as in species within the same order. In the case of a mammalian reference sequence, the amino acids that may be mutated in a preferred embodiment are those that are not conserved between the reference sequence and the orthologous sequence in another mammal species. For example, if a polypeptide used in a method of the present invention is said to comprise an amino acid sequence that is at least 90% identical to the IgV domain of human TIM-3, then said polypeptide may have non-identical residues to those positions in which the IgV domain of TIM-3 and that of mouse, cattle and/or orangutan differ. In another embodiment, the polypeptide used in a method of the present invention is at least 90% identical to the IgV domain of human TIM-3 and comprising one or more substitutions in the following positions: 32, 35, 39, 41-47, 51, 57, 60-62, 64-67, 72, 74, 76-79, 83, 85-86, 88-89, 94, 96, 98, 103, 105, 107, 114, 117, 121, 123-124, 126, and 128.

FIG. 8 depicts an amino acid sequence alignment of the IgV domain of TIM-3 homologs from human (SEQ ID NO:1), Pongo pygmaeus (SEQ ID NO:2, Genbank Accession No. CAH92001), Mus musculus (SEQ ID NO:3, Genbank Accession No. NP_{—}599011), and Bos taurus (SEQ ID NO:4, Genbank Accession No. NP_{—}001070573) As is apparent from FIG. 8, there are multiple residues along the IgV domain of TIM-3 that are not conserved amongst the mammalian TIM-3 polypeptides, including positions 22-25, 27-29, 32, 35, 39, 41-47, 51, 57, 60-62, 64-67, 72, 74, 76-79, 83, 85-86, 88-89, 94, 96, 98, 103, 105, 107, 114, 117, 121, 123-124, 126, 128, and 131, numbered according to SEQ ID NO:1. Polypeptides sharing at least 90% identity with the IgV domain of TIM-3 includes polypeptides having conservative substitutions in these areas of divergence. Typically seen as conservative substitutions are the replacements, one for another, among the aliphatic amino acids Ala, Val, Leu, and Ile, interchange of the hydroxyl residues Ser and Thr, exchange of the acidic residues Asp and Glu, substitution between the amide residues Asn and Gln, exchange of the basic residues Lys and Arg and replacements among the aromatic residues Phe, Tyr. Additional guidance concerning which amino acid changes are likely to be phenotypically silent are found in Bowie et al., Science 247:1306-1310 (1990).

Polypeptides that are at least 90%, at least 95%, at least 97%, at least 99% or 100% identical to amino acids 30-128 of SEQ ID NO: 1 preferably retain the function of the TIM-3 IgV domain. In some embodiments the polypeptides that are at least 90%, at least 95%, at least 97%, at least 99% or 100% identical to amino acids 30-128 of SEQ ID NO: 1 bind to a TIM-3 ligand and/or modulate activation of APCs. In some embodiments the TIM-3 ligand is galectin-9. In some embodiments the polypeptides that are at least 90% identical to amino acids 30-128 of SEQ ID NO: 1 inhibit the release of TNF-α by APCs, in particular CD11b⁺ microglia cells

Polypeptides that are at least 90% identical to SEQ ID NO: 5 preferably retain the ability to bind TIM-3 and/or modulate activation of APCs. In some embodiments the polypeptides that are at least 90% identical to SEQ ID NO: 5 stimulate the release of TNF-α by APCs

### C. Modified Polypeptides Having Amino Acid Identity to Reference Sequences

The invention further encompasses fusion polypeptides comprising a TIM-3 or galectin-9 polypeptide or fragments thereof, and a heterologous polypeptide. In one embodiment, the soluble TIM-3 polypeptide comprises the IgV domain but lacks at least part of the mucin domain, and lacks the transmembrane, and optionally the intracellular domain. In certain embodiments, fusion polypeptides comprising a soluble TIM-3 or a galectin 9 polypeptide and an immunoglobulin element are provided. An exemplary immunoglobulin element is a constant region like the Fc domain of a human IgG1 heavy chain (Browning et al., J. Immunol., 154, pp. 33-46 (1995)). Soluble receptor-IgG fusion polypeptides are common immunological reagents and methods for their construction are known in the art (see e.g., U.S. Pat. Nos. 5,225,538, 5,766,883 and 5,876,969), all of which are incorporated by reference. In some embodiments, soluble peptides of the present invention are fused to Fc variants.

In a related embodiment, the modified polypeptides of the invention comprise TIM-3 or galectin 9 fusion polypeptides with an Fc region of an immunoglobulin. As is known, each immunoglobulin heavy chain constant region comprises four or five domains. The domains are named sequentially as follows: CH1-hinge-CH2-CH3(-CH4). The DNA sequences of the heavy chain domains have cross-homology among the immunoglobulin classes, e.g., the CH2 domain of IgG is homologous to the CH2 domain of IgA and IgD, and to the CH3 domain of IgM and IgE. As used herein, the term, “immunoglobulin Fc region” is understood to mean the carboxyl-terminal portion of an immunoglobulin chain constant region, preferably an immunoglobulin heavy chain constant region, or a portion thereof. For example, an immunoglobulin Fc region may comprise 1) a CH1 domain, a CH2 domain, and a CH3 domain, 2) a CH1 domain and a CH2 domain, 3) a CH1 domain and a CH3 domain, 4) a CH2 domain and a CH3 domain, or 5) a combination of two or more domains and an immunoglobulin hinge region. In a preferred embodiment the immunoglobulin Fc region comprises at least an immunoglobulin hinge region a CH2 domain and a CH3 domain, and preferably lacks the CH1 domain.

In one embodiment, the class of immunoglobulin from which the heavy chain constant region is derived is IgG (Igγ) (γ subclasses 1, 2, 3, or 4). Other classes of immunoglobulin, IgA (Igα), IgD (Igδ), IgE (Igε) and IgM (Igμ), may be used. The choice of appropriate immunoglobulin heavy chain constant regions is discussed in detail in U.S. Pat. Nos. 5,541,087, and 5,726,044. The choice of particular immunoglobulin heavy chain constant region sequences from certain immunoglobulin classes and subclasses to achieve a particular result is considered to be within the level of skill in the art. The portion of the DNA construct encoding the immunoglobulin Fc region preferably comprises at least a portion of a hinge domain, and preferably at least a portion of a CH₃ domain of Fc γ or the homologous domains in any of IgA, IgD, IgE, or IgM.

Furthermore, it is contemplated that substitution or deletion of amino acids within the immunoglobulin heavy chain constant regions may be useful in the practice of the invention. One example would be to introduce amino acid substitutions in the upper CH2 region to create a Fc variant with reduced affinity for Fc receptors (Cole et al. (1997) J. IMMUNOL. 159:3613). One of ordinary skill in the art can prepare such constructs using well known molecular biology techniques.

In a further embodiment, the fusion polypeptides comprise a soluble TIM-3 or a galectin 9 polypeptide and a second heterologous polypeptide to increase the in vivo stability of the fusion polypeptide, or to modulate its biological activity or localization, or to facilitate purification of the fusion polypeptide. Other exemplary heterologous polypeptides that can be used to generate TIM-3 or galectin 9 soluble fusion polypeptides include, but not limited to, polyhistidine, Glu-Glu, glutathione S transferase (GST), thioredoxin, polypeptide A, polypeptide G, and an immunoglobulin heavy chain constant region (Fc), maltose binding polypeptide (MBP), which are particularly useful for isolation of the fusion polypeptides by affinity chromatography. For the purpose of affinity purification, relevant matrices for affinity chromatography, such as glutathione-, amylase-, and nickel- or cobalt-conjugated resins are used. Another fusion domain well known in the art is green fluorescent polypeptide (GFP). Fusion domains also include “epitope tags,” which are usually short peptide sequences for which a specific antibody is available. Well known epitope tags for which specific monoclonal antibodies are readily available include FLAG, influenza virus haemagglutinin (HA), and c-myc tags. In some cases, the fusion domains have a protease cleavage site, such as for Factor Xa or Thrombin, which allows the relevant protease to partially digest the fusion polypeptides and thereby liberate the recombinant polypeptides therefrom. The liberated polypeptides can then be isolated from the fusion domain by subsequent chromatographic separation.

Preferably, stable plasma polypeptides, which typically have a half-life greater than 20 hours in the circulation, are used to construct fusions polypeptides with TIM-3. Such plasma polypeptides include but are not limited to: immunoglobulins, serum albumin, lipopolypeptides, apolipopolypeptides and transferrin. Sequences that can target the soluble TIM-3 or galectin 9 molecules to a particular cell or tissue type may also be attached to the soluble TIM-3 or galectin 9 to create a specifically-localized soluble TIM-3 or galectin 9 fusion polypeptide.

In one preferred embodiment, the invention provides TIM-3 or galectin 9 fusions to albumin. As used herein, “albumin” refers collectively to albumin polypeptide or amino acid sequence, or an albumin fragment or variant, having one or more functional activities (e.g., biological activities) of albumin. In particular, “albumin” refers to human serum albumin or fragments thereof (see EP 201239, EP 322094 WO 97/24445, WO95/23857) especially the mature form of human albumin, or albumin from other vertebrates. In particular, the albumin fusion polypeptides of the invention may include naturally occurring polymorphic variants of human albumin and fragments of human albumin (See WO95/23857), for example those fragments disclosed in EP 322094 (namely HA (Pₙ), where n is 369 to 419). The albumin may be derived from any vertebrate, especially any mammal, for example human, cow, sheep, or pig. Non-mammalian albumins include, but are not limited to, hen and salmon. The albumin portion of the albumin fusion polypeptide may be from a different animal than the TIM-3 or galectin-9 polypeptide.

In some embodiments, the albumin polypeptide portion of an albumin fusion polypeptide corresponds to a fragment of serum albumin. Fragments of serum albumin polypeptides include polypeptides having one or more residues deleted from the amino terminus or from the C-terminus. Generally speaking, an HA fragment or variant will be at least 100 amino acids long, preferably at least 150 amino acids long. The HA variant may consist of or alternatively comprise at least one whole domain of HA. Domains, of human albumin are described in U.S. Patent Publication No. 2004/0171123.

It is also possible to modify the structure of the subject TIM-3 or galectin 9 polypeptides for such purposes as enhancing therapeutic or prophylactic efficacy, or stability (e.g., ex vivo shelf life and resistance to proteolytic degradation in vivo). Such modified polypeptides, when designed to retain at least one activity of the naturally-occurring form of the polypeptide, are considered functional equivalents of the TIM-3 or galectin 9 polypeptides described in more detail herein. Such modified polypeptides can be produced, for instance, by amino acid substitution, deletion, or addition.

For instance, it is reasonable to expect, for example, that an isolated replacement of a leucine with an isoleucine or valine, an aspartate with a glutamate, a threonine with a serine, or a similar replacement of an amino acid with a structurally related amino acid (i.e. conservative mutations) will not have a major effect on the biological activity of the resulting molecule. Conservative replacements are those that take place within a family of amino acids that are related in their side chains. Genetically encoded amino acids are can be divided into four families: (1) acidic=aspartate, glutamate; (2) basic=lysine, arginine, histidine; (3) nonpolar=alanine, valine, leucine, isoleucine, proline, phenylalanine, methionine, tryptophan; and (4) uncharged polar=glycine, asparagine, glutamine, cysteine, serine, threonine, tyrosine. Phenylalanine, tryptophan, and tyrosine are sometimes classified jointly as aromatic amino acids. In similar fashion, the amino acid repertoire can be grouped as (1) acidic=aspartate, glutamate; (2) basic=lysine, arginine histidine, (3) aliphatic=glycine, alanine, valine, leucine, isoleucine, serine, threonine, with serine and threonine optionally be grouped separately as aliphatic-hydroxyl; (4) aromatic=phenylalanine, tyrosine, tryptophan; (5) amide=asparagine, glutamine; and (6) sulfur-containing=cysteine and methionine. (see, for example, Biochemistry, 2nd ed., Ed. by L. Stryer, W.H. Freeman and Co., 1981). Whether a change in the amino acid sequence of a polypeptide results in a functional homolog can be readily determined by assessing the ability of the variant polypeptide to produce a response in cells in a fashion similar to the wild-type polypeptide. For instance, such variant forms of a TIM-3 or galectin 9 polypeptide can be assessed, e.g., for their ability to modulate the secretion of TNF-α by CD11b⁺ microglia cells. Polypeptides in which more than one replacement has taken place can readily be tested in the same manner.

Some of the TIM-3 or galectin 9 polypeptides provided by the invention, or used in the methods of the present invention, may further comprise post-translational modifications. Exemplary post-translational polypeptide modifications include phosphorylation, acetylation, methylation, ADP-ribosylation, ubiquitination, glycosylation, carbonylation, sumoylation, biotinylation or addition of a polypeptide side chain or of a hydrophobic group. As a result, the modified soluble polypeptides may contain non-amino acid elements, such as lipids, poly- or mono-saccharide, and phosphates.

A chimeric or fusion polypeptide for use in the present invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary-overhangs between two consecutive gene fragments that can subsequently be annealed and reamplified to generate a chimeric gene sequence (see, for example, Ausubel et al. (eds.) CURRENT PROTOCOLS IN MOLECULAR BIOLOGY, John Wiley & Sons, 1992). Moreover, many expression vectors are commercially available that encode a fusion moiety (e.g., an Fc region of an immunoglobulin heavy chain). A TIM-3 or galectin-9 encoding nucleic acid can be cloned into such an expression vector such that the fusion moiety is linked in-frame to the immunoglobulin polypeptide.

In one specific embodiment of the present invention, modified forms of the subject TIM-3 or galectin 9 polypeptides, comprise linking the subject soluble polypeptides to nonpolypeptide polymers. In one specific embodiment, the polymer is polyethylene glycol (“PEG”), polypropylene glycol, or polyoxyalkylenes, in the manner as set forth in U.S. Pat. No. 4,640,835; 4,496,689; 4,301,144; 4,670,417; 4,791,192 or 4,179,337. PEG is a well-known, water soluble polymer that is commercially available or can be prepared by ring-opening polymerization of ethylene glycol according to methods well known in the art (Sandler and Karo, Polymer Synthesis, Academic Press, New York, Vol. 3, pages 138-161). The term “PEG” is used broadly to encompass any polyethylene glycol molecule, without regard to size or to modification at an end of the PEG, and can be represented by the formula: X—O(CH₂CH₂O)ₙ₋₁CH₂CH₂OH (1), where n is 20 to 2300 and X is H or a terminal modification, e.g., a C₁₋₄ alkyl. In one embodiment, the PEG of the invention terminates on one end with hydroxy or methoxy, i.e., X is H or CH3 (“methoxy PEG”). A PEG can contain further chemical groups which are necessary for binding reactions; which results from the chemical synthesis of the molecule; or which is a spacer for optimal distance of parts of the molecule. In addition, such a PEG can consist of one or more PEG side-chains which are linked together. PEGs with more than one PEG chain are called multiarmed or branched PEGs. Branched PEGs can be prepared, for example, by the addition of polyethylene oxide to various polyols, including glycerol, pentaerythriol, and sorbitol. For example, a four-armed branched PEG can be prepared from pentaerythriol and ethylene oxide. Branched PEG are described in, for example, EP-0473084 and U.S. Pat. No. 5,932,462. One form of PEGs includes two PEG side-chains (PEG2) linked via the primary amino groups of a lysine (Monfardini, C., et al., Bioconjugate Chem. 6 (1995) 62-69).

PEG conjugation to peptides or polypeptides generally involves the activation of PEG and coupling of the activated PEG-intermediates directly to target polypeptides/peptides or to a linker, which is subsequently activated and coupled to target polypeptides/peptides (see Abuchowski, A. et al, J. Biol. Chem., 252, 3571 (1977) and J. Biol. Chem., 252, 3582 (1977), Zalipsky, et al., and Harris et. al., in: Poly(ethylene glycol) Chemistry: Biotechnical and Biomedical Applications; (J. M. Harris ed.) Plenum Press: New York, 1992; Chap. 21 and 22).

One skilled in the art can select a suitable molecular mass for PEG, e.g., based on how the pegylated TIM-3 or galectin 9 polypeptide will be used therapeutically, the desired dosage, circulation time, resistance to proteolysis, immunogenicity, and other considerations. For a discussion of PEG and its use to enhance the properties of polypeptides, see N. V. Katre, Advanced Drug Delivery Reviews 10: 91-114 (1993).

In one embodiment of the invention, PEG molecules may be activated to react with amino groups on TIM-3 or galectin 9 polypeptides, such as with lysines (Bencham C. O. et al., Anal. Biochem., 131, 25 (1983); Veronese, F. M. et al., Appl. Biochem., 11, 141 (1985); Zalipsky, S. et al., Polymeric Drugs and Drug Delivery Systems, adrs 9-110 ACS Symposium Series 469 (1999); Zalipsky, S. et al., Europ. Polym. J., 19, 1177-1183 (1983); Delgado, C. et al., Biotechnology and Applied Biochemistry, 12, 119-128 (1990)). In another embodiment, PEG molecules may be coupled to sulfhydryl groups on tim-4 or tim-1 (Sartore, L., et al., Appl. Biochem. Biotechnol., 27, 45 (1991); Morpurgo et al., Biocon. Chem., 7, 363-368 (1996); Goodson et al., Bio/Technology (1990) 8, 343; U.S. Pat. No. 5,766,897). U.S. Pat. Nos. 6,610,281 and 5,766,897 describes exemplary reactive PEG species that may be coupled to sulfhydryl groups. In some embodiments, the pegylated TIM-3 or galectin 9 polypeptides comprise a PEG molecule covalently attached to the alpha amino group of the N-terminal amino acid. Site specific N-terminal reductive amination is described in Pepinsky et al., (2001) JPET, 297, 1059, and U.S. Pat. No. 5,824,784. The use of a PEG-aldehyde for the reductive amination of a polypeptide utilizing other available nucleophilic amino groups is described in U.S. Pat. No. 4,002,531, in Wieder et al., (1979) J. Biol. Chem. 254, 12579, and in Chamow et al., (1994) Bioconjugate Chem. 5, 133.

### IV. Agents that Modulate TIM-3 Activity

As used herein, “TIM-3 activity” refers to a signalling activity mediated by TIM-3, and includes activation of downstream effectors of TIM-3 as well as modulation of inflammatory cytokine expression by T-cells and/or TIM-3 dependent activation of APCs. Thus, TIM-3 activity can be measured by measuring TIM-3 dependent changes in downstream effectors of TIM-3.

In one embodiment, the agent that decreases TIM-3 activity inhibits binding of galectin-9 to TIM-3. In one embodiment, the agent which inhibits binding of full-length TIM-3 to galectin-9 comprises a carbohydrate, such as lactose or pectin/modified pectin. Modified pectins are described in U.S. Patent Pub. Nos. 2003/0004132 and 2002/0187959.

In other embodiments, the agent which increases TIM-3 activity is a peptide mimetic or a small molecule which can functionally replace galectin-9 in activating the TIM-3 receptor. The peptide or small molecule can structurally resemble the surface of galectin-9 that binds TIM-3, such that the peptide or small molecule can activate TIM-3 upon binding it, leading to increased TIM-3 activity. In a specific embodiment, the agent which increases TIM-3 activity promotes the tyrosine phosphorylation of the intracellular domain of TIM-3.

As used herein, TIM-3 activity is “decreased” if one or more signalling activities or downstream read-outs of TIM-3 activity is reduced by a statistically significant amount, and preferably by at least 10% in the presence of an agent or stimulus relative to the absence of such agent or stimulus.

As used herein, TIM-3 activity is “increased” if one or more signalling activities or downstream read-outs of TIM-3 activity is increased by a statistically significant amount, and preferably by at least 10% in the presence of an agent or stimulus, relative to the absence of such agent or stimulus.

### A. Antisense Oligonucleotides

In some embodiments, TIM-3 activity is modulated with TIM-3 or galectin-9 antagonists. In some embodiments, these antagonists comprise an RNAi/antisense oligonucleotide such as a double stranded RNA molecule or a DNA construct capable of generating double stranded RNA. In yet another embodiment, the agent which increases TIM-3 activity reduces the expression or function of soluble TIM-3, but does not directly affect that of full-length TIM-3. In one embodiment, the agent is a double stranded RNA species which specifically inhibits the expression of soluble TIM-3, such as the one comprising the nucleotide sequence according to SEQ ID NO: 6. Double stranded RNA includes, but is not limited to, hairpin RNA and RNA formed by two complementary single stranded RNA molecules. Antisense oligonucleotides are relatively short nucleic acids that are complementary (or antisense) to the coding strand (sense strand) of the mRNA encoding a particular polypeptide. Although antisense oligonucleotides are typically RNA based, they can also be DNA based. Additionally, antisense oligonucleotides are often modified to increase their stability.

Without being bound by theory, the binding of these relatively short oligonucleotides to the mRNA is believed to induce stretches of double stranded RNA that trigger degradation of the messages by endogenous RNAses. Additionally, sometimes the oligonucleotides are specifically designed to bind near the promoter of the message, and under these circumstances, the antisense oligonucleotides may additionally interfere with translation of the message. Regardless of the specific mechanism by which antisense oligonucleotides function, their administration to a cell, tissue or organism allows the degradation of the mRNA encoding a specific polypeptide. Accordingly, antisense oligonucleotides decrease the expression and/or activity of a particular polypeptide. In this case, they would be specifically desired to target TIM-3 and/or galectin-9.

The oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors), or compounds facilitating transport across the cell membrane (see, e.g., Letsinger et al., 1989, Proc. Natl. Acad. Sci. USA. 86:6553-6556; Lemaitre et al., 1987, Proc. Natl. Acad. Sci. 84:648-652; PCT Publication No. WO88/09810, published Dec. 15, 1988) or the blood-brain barrier (see, e.g., PCT Publication No. WO89/10134, published Apr. 25, 1988), hybridization-triggered cleavage agents (See, e.g., Krol et al., 1988, BioTechniques 6:958-976) or intercalating agents. (See, e.g., Zon, 1988, Pharm. Res. 5:539-549). To this end, the oligonucleotide may be conjugated to another molecule.

The antisense oligonucleotide may comprise at least one modified base moiety which is selected from the group including but not limited to 5-fluorouracil, 5-bromouracil, 5-chlorouracil, 5-iodouracil, hypoxanthine, xanthine, 4-acetylcytosine, 5-(carboxyhydroxytriethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methyl ester, uracil-5-oxyacetic acid (v), 5-methyl-2-thiouracil, 3-(3-amino-3-N-2-carboxypropyl) uracil, (acp3)w, and 2,6-diaminopurine.

The antisense oligonucleotide may also comprise at least one modified sugar moiety selected from the group including but not limited to arabinose, 2-fluoroarabinose, xylulose, and hexose. The antisense oligonucleotide can also contain a neutral peptide-like backbone. Such molecules are termed peptide nucleic acid (PNA)-oligomers and are described, e.g., in Perry-O'Keefe et al. (1996) Proc. Natl. Acad. Sci. USA. 93:14670 and in Eglom et al. (1993) Nature 365:566. One advantage of PNA oligomers is their capability to bind to complementary DNA essentially independently from the ionic strength of the medium due to the neutral backbone of the DNA. In yet another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet a further embodiment, the antisense oligonucleotide is an α-anomeric oligonucleotide. An α-anomeric oligonucleotide forms specific double-stranded hybrids with complementary RNA in which, contrary to the usual α-units, the strands run parallel to each other (Gautier et al., 1987, Nucl. Acids Res. 15:6625-6641). The oligonucleotide can be a 2′-0-methylribonucleotide (Inoue et al., 1987, Nucl. Acids Res. 15:6131-6148), or a chimeric RNA-DNA analogue (Inoue et al., 1987, FEBS Lett. 215:327-330).

Oligonucleotides for use in the invention may be synthesized by standard methods known in the art, e.g., by use of an automated DNA synthesizer (such as are commercially available from Biosearch, Applied Biosystems, etc.). As examples, phosphorothioate oligonucleotides may be synthesized by the method of Stein et al. (1988, Nucl. Acids Res. 16:3209), methylphosphonate oligonucleotides can be prepared by use of controlled pore glass polymer supports (Sarin et al., 1988, Proc. Natl. Acad. Sci. USA. 85:7448-7451), etc.

The selection of an appropriate oligonucleotide can be readily performed by one of skill in the art. Given the nucleic acid sequence encoding a particular polypeptide, one of skill in the art can design antisense oligonucleotides that bind said sequence and test these oligonucleotides in an in vitro or in vivo system to confirm that they bind to and mediate the degradation of the mRNA encoding the particular polypeptide. To design an antisense oligonucleotide that specifically binds to and mediates the degradation of a particular mRNA, it is important that the sequence recognized by the oligonucleotide is unique or substantially unique to that particular mRNA. For example, sequences that are frequently repeated across mRNA may not be an ideal choice for the design of an oligonucleotide that specifically recognizes and degrades a particular message. One of skill in the art can design an oligonucleotide, and compare the sequence of that oligonucleotide to nucleic acid sequences that are deposited in publicly available databases to confirm that the sequence is specific or substantially specific for a particular polypeptide.

In another example, it may be desirable to design an antisense oligonucleotide that binds to and mediates the degradation of more than one message. In one example, the messages may encode related polypeptide such as isoforms or functionally redundant polypeptide. In such a case, one of skill in the art can align the nucleic acid sequences that encode these related polypeptides, and design an oligonucleotide that recognizes both messages.

A number of methods have been developed for delivering antisense DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systematically.

However, it may be difficult to achieve intracellular concentrations of the antisense sufficient to suppress translation on endogenous TIM-3 or galectin-9 mRNAs in certain instances. Therefore another approach utilizes a recombinant DNA construct in which the antisense oligonucleotide is placed under the control of a strong pol III or pol II promoter. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, preferably human cells. Such promoters can be inducible or constitutive. Such promoters include but are not limited to: the SV40 early promoter region (Bernoist and Chambon, 1981, Nature 290:304-310), the promoter contained in the 3′ long terminal repeat of Rous sarcoma virus (Yamamoto et al., 1980, Cell 22:787-797), the herpes thymidine kinase promoter (Wagner et al., 1981, Proc. Natl. Acad. Sci. USA. 78:1441-1445), the regulatory sequences of the metallothionein gene (Brinster et al, 1982, Nature 296:39-42), etc. Any type of plasmid, cosmid, YAC or viral vector can be used to prepare the recombinant DNA construct that can be introduced directly into the tissue site. Alternatively, viral vectors can be used which selectively infect the desired tissue, in which case administration may be accomplished by another route (e.g., systematically).

RNAi constructs comprise double stranded RNA that can specifically block expression of a target gene. “RNA interference” or “RNAi” is a term initially applied to a phenomenon observed in plants and worms where double-stranded RNA (dsRNA) blocks gene expression in a specific and post-transcriptional manner. Without being bound by theory, RNAi appears to involve mRNA degradation via activation of a specific set of nucleases, however the biochemical mechanisms are currently an active area of research. Despite some uncertainty regarding the mechanism of action, RNAi provides a useful method of inhibiting gene expression in vitro or in vivo. As used herein, the term “dsRNA” refers to siRNA molecules, or other RNA molecules including a double stranded feature and able to be processed to siRNA in cells, such as hairpin RNA moieties. The term “loss-of-function,” as it refers to genes inhibited by the subject RNAi method, refers to a diminishment in the level of expression of a gene when compared to the level in the absence of RNAi constructs.

As used herein, the phrase “mediates RNAi” refers to (indicates) the ability to distinguish which RNAs are to be degraded by the RNAi process, e.g., degradation occurs in a sequence-specific manner rather than by a sequence-independent dsRNA response, e.g., a PKR response.

As used herein, the term “RNAi construct” is a generic term used throughout the specification to include small interfering RNAs (siRNAs), hairpin RNAs, and other RNA species which can be cleaved in vivo to form siRNAs. RNAi constructs herein also include expression vectors (also referred to as RNAi expression vectors) capable of giving rise to transcripts which form dsRNAs or hairpin RNAs in cells, and/or transcripts which can produce siRNAs in vivo.

“RNAi expression vector” (also referred to herein as a “dsRNA-encoding plasmid”) refers to replicable nucleic acid constructs used to express (transcribe) RNA which produces siRNA moieties in the cell in which the construct is expressed. Such vectors include a transcriptional unit comprising an assembly of (1) genetic element(s) having a regulatory role in gene expression, for example, promoters, operators, or enhancers, operatively linked to (2) a “coding” sequence which is transcribed to produce a double-stranded RNA (two RNA moieties that anneal in the cell to form an siRNA, or a single hairpin RNA which can be processed to an siRNA), and (3) appropriate transcription initiation and termination sequences. The choice of promoter and other regulatory elements generally varies according to the intended host cell. In general, expression vectors of utility in recombinant DNA techniques are often in the form of “plasmids” which refer to circular double stranded DNA loops which, in their vector form are not bound to the chromosome. In the present specification, “plasmid” and “vector” are used interchangeably as the plasmid is the most commonly used form of vector. However, the invention is intended to include such other forms of expression vectors, e.g., viral vectors or others, which serve equivalent functions and vectors which become known in the art subsequently hereto.

The RNAi constructs contain a nucleotide sequence that hybridizes under physiologic conditions of the cell to the nucleotide sequence of at least a portion of the mRNA transcript for the gene to be inhibited (i.e., the “target” gene, e.g., TIM-3). The double-stranded RNA need only be sufficiently similar to natural RNA that it has the ability to mediate RNAi. Thus, the invention has the advantage of being able to tolerate sequence variations that might be expected due to genetic mutation, strain polymorphism or evolutionary divergence. The number of tolerated nucleotide mismatches between the target sequence and the RNAi construct sequence is no more than 1 in 5 basepairs, or 1 in 10 basepairs, or 1 in 20 basepairs, or 1 in 50 basepairs. Mismatches in the center of the siRNA duplex are most critical and may essentially abolish cleavage of the target RNA. In contrast, nucleotides at the 3′ end of the siRNA strand that is complementary to the target RNA do not significantly contribute to specificity of the target recognition.

Sequence identity can be optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). Greater than 90% sequence identity, or even 100% sequence identity, between the inhibitory RNA and the portion of the target gene is preferred. Alternatively, the duplex region of the RNA can be defined functionally as a nucleotide sequence that is capable of hybridizing with a portion of the target gene transcript (e.g., 400 mM NaCl, 40 mM PIPES pH 6.4, 1 mM EDTA, 50° C. or 70° C. hybridization for 12-16 hours; followed by washing).

Production of RNAi constructs can be carried out by chemical synthetic methods or by recombinant nucleic acid techniques. Endogenous RNA polymerase of the treated cell can mediate transcription in vivo, or cloned RNA polymerase can be used for transcription in vitro. The RNAi constructs can include modifications to either the phosphate-sugar backbone or the nucleoside, e.g., to reduce susceptibility to cellular nucleases, improve bioavailability, improve formulation characteristics, and/or change other pharmacokinetic properties. For example, the phosphodiester linkages of natural RNA may be modified to include at least one of an nitrogen or sulfur heteroatom. Modifications in RNA structure can be tailored to allow specific genetic inhibition while avoiding a general response to dsRNA. Likewise, bases can be modified to block the activity of adenosine deaminase. The RNAi construct can be produced enzymatically or by partial/total organic synthesis; any modified ribonucleotide can be introduced by in vitro enzymatic or organic synthesis.

Methods of chemically modifying RNA molecules can be adapted for modifying RNAi constructs (see, for example, Heidenreich et al. (1997) Nucleic Acids Res, 25:776-780; Wilson et al. (1994) J Mol Recog 7:89-98; Chen et al. (1995) Nucleic Acids Res 23:2661-2668; Hirschbein et al. (1997) Antisense Nucleic Acid Drug Dev 7:55-61). Merely to illustrate, the backbone of an RNAi construct can be modified with phosphorothioates, phosphoramidate, phosphodithioates, chimeric methylphosphonate-phosphodiesters, peptide nucleic acids, 5-propynyl-pyrimidine containing oligomers or sugar modifications (e.g., 2′-substituted ribonucleosides, a-configuration).

The double-stranded structure may be formed by a single self-complementary RNA strand or two complementary RNA strands. RNA duplex formation may be initiated either inside or outside the cell. The RNA may be introduced in an amount which allows delivery of at least one copy per cell. Higher doses (e.g., at least 5, 10, 100, 500 or 1000 copies per cell) of double-stranded material may yield more effective inhibition, while lower doses may also be useful for specific applications. Inhibition is sequence-specific in that nucleotide sequences corresponding to the duplex region of the RNA are targeted for genetic inhibition.

In certain embodiments, the subject RNAi constructs are “small interfering RNAs” or “siRNAs.” These nucleic acids are around 19-30 nucleotides in length, and even more preferably 21-23 nucleotides in length, e.g., corresponding in length to the fragments generated by nuclease “dicing” of longer double-stranded RNAs. The siRNAs are understood to recruit nuclease complexes and guide the complexes to the target mRNA by pairing to the specific sequences. As a result, the target mRNA is degraded by the nucleases in the polypeptide complex. In a particular embodiment, the 21-23 nucleotides siRNA molecules comprise a 3′ hydroxyl group.

The siRNA molecules of the present invention can be obtained using a number of techniques known to those of skill in the art. For example, the siRNA can be chemically synthesized or recombinantly produced using methods known in the art. For example, short sense and antisense RNA oligomers can be synthesized and annealed to form double-stranded RNA structures with 2-nucleotide overhangs at each end (Caplen, et al. (2001) Proc Natl Acad Sci USA, 98:9742-9747; Elbashir, et al. (2001) EMBO J, 20:6877-88). These double-stranded siRNA structures can then be directly introduced to cells, either by passive uptake or a delivery system of choice, such as described below.

In certain embodiments, the siRNA constructs can be generated by processing of longer double-stranded RNAs, for example, in the presence of the enzyme dicer. In one embodiment, the Drosophila in vitro system is used. In this embodiment, dsRNA is combined with a soluble extract derived from Drosophila embryo, thereby producing a combination. The combination is maintained under conditions in which the dsRNA is processed to RNA molecules of about 21 to about 23 nucleotides.

The siRNA molecules can be purified using a number of techniques known to those of skill in the art. For example, gel electrophoresis can be used to purify siRNAs. Alternatively, non-denaturing methods, such as non-denaturing column chromatography, can be used to purify the siRNA. In addition, chromatography (e.g., size exclusion chromatography), glycerol gradient centrifugation, affinity purification with antibody can be used to purify siRNAs.

In certain preferred embodiments, at least one strand of the siRNA molecules has a 3′ overhang from about 1 to about 6 nucleotides in length, though may be from 2 to 4 nucleotides in length. More preferably, the 3′ overhangs are 1-3 nucleotides in length. In certain embodiments, one strand having a 3′ overhang and the other strand being blunt-ended or also having an overhang. The length of the overhangs may be the same or different for each strand. In order to further enhance the stability of the siRNA, the 3′ overhangs can be stabilized against degradation. In one embodiment, the RNA is stabilized by including purine nucleotides, such as adenosine or guanosine nucleotides. Alternatively, substitution of pyrimidine nucleotides by modified analogues, e.g., substitution of uridine nucleotide 3′ overhangs by 2′-deoxythyinidine is tolerated and does not affect the efficiency of RNAi. The absence of a 2′ hydroxyl significantly enhances the nuclease resistance of the overhang in tissue culture medium and may be beneficial in vivo.

In other embodiments, the RNAi construct is in the form of a long double-stranded RNA. In certain embodiments, the RNAi construct is at least 25, 50, 100, 200, 300 or 400 bases. In certain embodiments, the RNAi construct is 400-800 bases in length. The double-stranded RNAs are digested intracellularly, e.g., to produce siRNA sequences in the cell. However, use of long double-stranded RNAs in vivo is not always practical, presumably because of deleterious effects which may be caused by the sequence-independent dsRNA response. In such embodiments, the use of local delivery systems and/or agents which reduce the effects of interferon or PKR are preferred.

In certain embodiments, the RNAi construct is in the form of a hairpin structure (named as hairpin RNA). The hairpin RNAs can be synthesized exogenously or can be formed by transcribing from RNA polymerase III promoters in vivo. Examples of making and using such hairpin RNAs for gene silencing in mammalian cells are described in, for example, Paddison et al., Genes Dev, 2002, 16:948-58; McCaffrey et al., Nature, 2002, 418:38-9; McManus et al., RNA, 2002, 8:842-50; Yu et al., Proc Natl Acad Sci USA, 2002, 99:6047-52). Preferably, such hairpin RNAs are engineered in cells or in an animal to ensure continuous and stable suppression of a desired gene. It is known in the art that siRNAs can be produced by processing a hairpin RNA in the cell.

In yet other embodiments, a plasmid is used to deliver the double-stranded RNA, e.g., as a transcriptional product. In such embodiments, the plasmid is designed to include a “coding sequence” for each of the sense and antisense strands of the RNAi construct. The coding sequences can be the same sequence, e.g., flanked by inverted promoters, or can be two separate sequences each under transcriptional control of separate promoters. After the coding sequence is transcribed, the complementary RNA transcripts base-pair to form the double-stranded RNA.

PCT application WO01/77350 describes an exemplary vector for bi-directional transcription of a transgene to yield both sense and antisense RNA transcripts of the same transgene in a eukaryotic cell. Accordingly, in certain embodiments, the present invention provides a recombinant vector having the following unique characteristics: it comprises a viral replicon having two overlapping transcription units arranged in an opposing orientation and flanking a transgene for an RNAi construct of interest, wherein the two overlapping transcription units yield both sense and antisense RNA transcripts from the same transgene fragment in a host cell.

RNAi constructs can comprise either long stretches of double stranded RNA identical or substantially identical to the target nucleic acid sequence or short stretches of double stranded RNA identical to substantially identical to only a region of the target nucleic acid sequence. Exemplary methods of making and delivering either long or short RNAi constructs can be found, for example, in WO01/68836 and WO01/75164.

### B. Ribozyme Molecules

In another embodiment, the TIM-3 or galectin-9 antagonists are ribozyme molecules which reduce the expression levels of TIM-3 or galectin-9. Ribozyme molecules designed to catalytically cleave an mRNA transcript can be used to prevent translation of TIM-3 or galectin-9 mRNA (See, e.g., PCT International Publication WO90/11364, published Oct. 4, 1990; Sarver et al., 1990, Science 247:1222-1225 and U.S. Pat. No. 5,093,246). While ribozymes that cleave mRNA at site-specific recognition sequences can be used to destroy particular mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Haseloff and Gerlach, 1988, Nature, 334:585-591.

### C. Antibodies

In another embodiment of the methods described herein, the agent that decreases or increases TIM-3 activity comprises an anti-TIM-3 antibody. Antibodies which bind the TIM-3 extracellular domain, such as monoclonal antibodies, can be generated by one skilled in the art, and those antibodies can be further tested for their ability to block binding of TIM-3 and TIM-3 ligands using the methods provided by the instant invention. Antagonist antibodies block the binding interactions between TIM-3 and TIM-3 ligands (e.g. galectin-9) without themselves acting as an activator of TIM-3 activity. In one embodiment, the antibody binds to the IgV domain of TIM-3 (amino acids 30-128 of SEQ ID NO:1) and blocks binding of TIM-3 ligands to full-length TIM-3. One skilled in the art can determine if a candidate antibody is an antagonist of TIM-3 activity by using assays to monitor a reduced Th1 response or an increase in Th2 response. Such testing, for example, can be performed by administering the antibody to an immunized mouse and testing for in vitro proliferation and cytokine production by T cells isolated from the spleen of the mouse.

In other embodiments, agonist antibodies to TIM-3 are used. Antibodies can be generated which bind to TIM-3 and mimic the binding of a ligand, resulting in intracellular signaling. Upon generating an antibody that binds to the extracellular domain of TIM-3, a skilled artisan may test whether the antibody activates TIM-3, which would lead to a suppression of Th1 cell proliferation, reduced cytokine release, and increased tolerance. The methods described in the experimental procedure can be used to determine if antibody binding activates full-length TIM-3. Activation of TIM-3 may be monitored, for example, by monitoring the intracellular tyrosine phosphorylation of TIM-3. In a specific embodiment, the antibody which increases TIM-3 activity is a bispecific antibody specific for TIM-3 and galectin-9.

The term “antibody” as used herein refers to immunoglobulin molecules and immunologically active portions of immunoglobulin (Ig) molecules, i.e., molecules that contain an antigen binding site that specifically binds (immunoreacts with) an antigen. Such antibodies include, e.g., polyclonal, monoclonal, chimeric, single chain, F_{ab}, F_{ab}′ and F(ab′)₂ fragments, and an Fab expression library. In general, an antibody molecule obtained from humans relates to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain. Reference herein to antibodies includes a reference to all such classes, subclasses and types of human antibody species. Antibodies to TIM-3 polypeptides also include antibodies to fusion polypeptides containing TIM-3 polypeptides or fragments of TIM-3 polypeptides.

A TIM-3 polypeptide can be used as an antigen, or a portion or fragment thereof, and additionally can be used as an immunogen to generate antibodies that immunospecifically bind the antigen, using standard techniques for polyclonal and monoclonal antibody preparation. Antigenic peptide fragments of the antigen for use as immunogens include, e.g., at least 7 amino acid residues of the amino acid sequence of the amino terminal region, such as an amino acid sequence shown in SEQ ID NO:1 and encompass an epitope thereof such that an antibody raised against the peptide forms a specific immune complex with the full length polypeptide or with any fragment that contains the epitope. Preferably, the antigenic peptide comprises at least 10 amino acid residues, or at least 15 amino acid residues, or at least 20 amino acid residues, or at least 30 amino acid residues. Preferred epitopes encompassed by the antigenic peptide are regions of the polypeptide that are located on its surface; commonly these are hydrophilic regions. In a preferred embodiment, the antigenic peptide comprises a segment of, or the entire, IgV and/or mucin domains of TIM-3.

In some embodiments, at least one epitope encompassed by the antigenic peptide is a region of TIM-3 polypeptide that is located on the surface of the polypeptide, e.g., a hydrophilic region. A hydrophobicity analysis of a TIM-3 polypeptide will indicate which regions of TIM-3 polypeptide are particularly hydrophilic and, therefore, are likely to encode surface residues useful for targeting antibody production. As a means for targeting antibody production, hydropathy plots showing regions of hydrophilicity and hydrophobicity can be generated by any method well known in the art, including, for example, the Kyte Doolittle or the Hopp Woods methods, either with or without Fourier transformation. See, e.g., Hopp and Woods (1981) Proc. Nat. Acad. Sci. USA 78: 3824-3828; Kyte and Doolittle (1982) J. Mol. Biol. 157: 105-142. Antibodies that are specific for one or more domains within an antigenic polypeptide, or derivatives, fragments, analogs or homologs thereof, are also provided herein. In some embodiments, a derivative, fragment, analog, homolog or ortholog of TIM-3 may be utilized as an immunogen in the generation of antibodies that immunospecifically bind these polypeptide components.

Various procedures known within the art may be used for the production of polyclonal or monoclonal antibodies directed against a polypeptide of the invention, or against derivatives, fragments, analogs homologs or orthologs thereof. See, for example, ANTIBODIES: A LABORATORY MANUAL, Harlow and Lane (1988) Cold Spring Harbor Laboratory Press, Cold Spring Harbor, N.Y. Some of these antibodies are discussed below.

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs thus contain an antigen binding site capable of immunoreacting with a particular epitope of the antigen characterized by a unique binding affinity for it.

Monoclonal antibodies can be prepared using hybridoma methods, such as those described by Kohler and Milstein (1975) Nature, 256:495. In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The antibodies directed against the polypeptide antigens of the invention can further comprise humanized antibodies or human antibodies. These, antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al. (1986) Nature, 321:522-525; Riechmann et al. (1988) Nature, 332:323-327; Verhoeyen et al. (1988) Science, 239:1534-1536), by substituting rodent CDRs or CDR sequences for the corresponding sequences of a human antibody. (See also U.S. Pat. No. 5,225,539.)

Antibody fragments that contain the idiotypes to a the TIM-3 may be produced by techniques known in the art including, but not limited to: (i) an F(ab′)₂ fragment produced by pepsin digestion of an antibody molecule; (ii) an Fab fragment generated by reducing the disulfide bridges of an F(ab′)₂ fragment; (iii) an Fab fragment generated by the treatment of the antibody molecule with papain and a reducing agent and (iv) Fv fragments.

### D. Method to Determine Effect on TIM-3 Activity

In some embodiments, an agent that decreases TIM-3 activity reduces the binding of TIM-3 to TIM-3 ligands. In one embodiment, the agent binds the extracellular domain of TIM-3 and prevents TIM-3 ligands from binding. In one embodiment, the agent is an anti-TIM-3 antagonist antibody. In one embodiment, the agent binds galectin-9. In one embodiment, the agent comprises a polypeptide comprising amino acids 30-128 of SEQ ID NO: 1 or an amino acid sequence that is at least 90% identical to amino acids 30-128 of SEQ ID NO: 1. In one embodiment the agent inhibits release of TNF-α in APCs. In one embodiment, the agent enhances IFN-γ secretion by CD4⁺ cells stimulated with anti-CD3 antibodies.

In some embodiments, the agent increases TIM-3 activity. In some embodiments, the agent binds TIM-3 and activates signaling. In some embodiments, the agent is a TIM-3 ligand. In some embodiments, the agent is a polypeptide comprising an amino acid sequence that is at least 90% identical to the amino acid sequence of SEQ ID NO:5. In some embodiments, the agent is an anti-TIM-3 agonist antibody. In some embodiments, the agent stimulates release of TNF-α by APCs. In one embodiment, the agent inhibits IFN-γ secretion by CD4⁺ cells stimulated with anti-CD3 antibodies. In one embodiment, the agent inhibits proliferation in CD4⁺ cells.

Simple binding assays can be used to detect agents which bind to TIM-3 or galectin-9 or disrupt the interaction between a TIM-3 polypeptide and a galectin-9 polypeptide. Because TIM-3 and galectin-9 are transmembrane proteins, assays that use the soluble forms of these proteins rather than full-length protein can be used. Soluble forms include those lacking the transmembrane domain and/or those comprising the IgV domain or fragments thereof which retain their ability to bind their cognate binding partners.

For example, a TIM-3 protein can be attached to the bottoms of wells in a multi-well plate (e.g., 96-well plate) by introducing a solution containing the protein into the plate and allowing the protein to bind to the plastic. The excess protein-containing solution is then washed out, and a blocking solution (containing, for example, bovine serum albumin (BSA)) is introduced to block non-specific binding sites. The plate is then washed several more times and a solution containing a galectin-9 protein and, in the case of experimental (vs. control) wells, an agent that effects TIM-3 activity. Alternatively, the wells of a multi-well plate may be coated with a polypeptide containing the galectin-9 protein, rather than the TIM-3 protein, and binding interactions assayed upon addition of a free TIM-3 protein. The wells may also be pre-coated with compound(s) that enhance attachment of the protein to be immobilized and/or decrease the level of non-specific binding. For example, the wells may be derivatized to contain glutathione and may be pre-coated with BSA, to promote attachment of the immobilized protein in a known orientation with the binding site(s) exposed.

As used herein, the term “reduces binding of galectin-9 to TIM-3” means that binding is reduced by at least 10% as measured in a TIM-3/galectin-9 binding assay as described herein (see the preceding two paragraphs).

In general, the term “inhibit binding” or “reduce binding” refers to a statistically significant reduction or inhibition of binding; to avoid doubt, however, the term generally refers to at least a 10% inhibition or reduction in binding.

Detection methods useful in such assays include antibody-based methods (i.e., an antibody directed against the “free” protein), direct detection of a reporter moiety incorporated into the “free” protein (such as a fluorescent label), and proximity energy transfer methods (such as a radioactive “free” protein resulting in fluorescence or scintillation of molecules incorporated into the immobilized protein or the solid support).

Yet another variation of assays to determine binding of a TIM-3 protein to a galectin-9 protein is through the use of affinity biosensor methods. Such methods may be based on the piezoelectric effect, electrochemistry, or optical methods, such as ellipsometry, optical wave guidance, and surface plasmon resonance (SPR).

Assays to determine the activity of TIM-3 are disclosed in US Application No. 2005/0191721. Examples 6-9 and 17 disclosed herein also describe assays to monitor TIM-3 activity. In an example of an ex vivo assay, human monocytes are isolated by negative selection from the peripheral blood of healthy subjects using magnetic beads (Miltenyi Biotech). Monocytes (2×10⁵/well) are stimulated with graded doses of the galectin-9 polypeptides and cytokine production is measured after 48 hours by ELISA and compared to cytokine production in monocytes stimulated with SEQ ID NO:5. Alternatively, monocytes are stimulated with SEQ ID NO:5 in the presence of TIM-3 polypeptides in order to determine if activation is affected.

### V. Methods of Treating Inflammatory Diseases or Disorders of the CNS

The invention is based in part on the surprising discovery that both TIM-3 and its ligand, galectin-9, are up-regulated on glial cells (microglia and astrocytes, respectively) in inflamed white matter tissue. Peripheral bone marrow-derived monocytes give rise to microglia, and peripheral monocytes infiltrate the CNS in a wide number of inflammatory CNS diseases. Thus, both resident microglia and the infiltrating monocytes from which they arise can contribute to CNS inflammation. Without wishing to be bound by theory, by regulating TIM-3 expression/activity on microglia, the inflammatory contribution of innate immunity to the CNS can be modulated.

One aspect of the invention provides a method for treating a nervous system disorder, the method comprising administering to a subject a therapeutically effective amount of an agent that decreases TIM-3 activity in the subject. The nervous system disorder includes, but is not limited to, inflammation in the central nervous system, demyelinating central nervous system diseases, encephalitis, meningitis, AIDS dementia, cerebral malaria, Alzheimer's disease, Parkinson's disease, multiple sclerosis (MS), and Huntington's disease. Each of these disorders has an inflammatory component that is expected to benefit from inhibition of TIM-3 activity. One aspect of the invention provides a method of treating multiple sclerosis in a subject, the method comprising administering to a subject a therapeutically effective amount of an agent that decreases TIM-3 activity in the subject.

In one embodiment, the method decreases TIM-3 activity in APCs. In one embodiment, the APCs are monocytes or CD11b⁺ microglia cells. In one embodiment, the APCs are DCs. In another embodiment the APCs are in the central nervous system.

In one embodiment, the MS being treated is classified as secondary progressive MS. It is routine for a person skilled in the art to diagnose subjects with a particular category of MS. Generally, symptoms are constant and do not improve in secondary progressive MS and are likely to become increasingly worse. While immunosuppression therapy, such as treatment with methotrexate, azathioprine, cyclophosphamide, or cladribine, has been shown to have affects in the early stages of MS, such as in relapsing remitting MS, subjects in secondary progressive stage are generally refractory to immunotherapy.

One aspect of the invention provides a method of prolonging life expectancy in a subject afflicted with multiple sclerosis, and in particular secondary progressive multiple sclerosis; the method comprising administering to a subject a therapeutically effective amount of an agent that decreases TIM-3 activity in the subject. In one embodiment, the agent that decreases TIM-3 activity prolongs the life expectancy of a subject by at least one day, at least one week, at least two weeks, at least three weeks, at least one month, at least two months, at least three months, at least 6 months, or at least one year. One aspect of the invention provides a method of prolonging health-adjusted life expectancy in a subject afflicted with multiple sclerosis, and in particular secondary progressive multiple sclerosis; the method comprising administering to a subject a therapeutically effective amount of an agent that decreases TIM-3 activity in the subject. In one embodiment, the agent that decreases TIM-3 activity prolongs the health-adjusted life expectancy of a subject by at least one day, at least one week, at least two weeks, at least three weeks, at least one month, at least two months, at least three months, at least 6 months, or at least one year.

Life expectancy can be defined as the average lifespan of a group of similar individuals, for example individuals suffering from the same disorder (Naimark D., et. al., J Gen Intern Med 9:702-707 (1994)). Factors that affect life expectancy include gender, genetic disorders, genetic background, obesity, health care, diet, age, and risky behaviors such as drug, alcohol, or tobacco use. Increase in life expectancy can be used as a means to measure and evaluate medical interventions intended to extend life (Wright J C and Weinstein M C, N Engl J Med, 339:380-386 (1998)).

Health-adjusted life expectancy is a measurement that accounts for both changes in mortality as well as changes in morbidity and disability and is an indicator of quality of life. Various measurements exist to assess quality of life and the effect of medical interventions on quality of life, such as years of potential life lost, disability-free life expectancy, health-adjusted life year, quality adjusted life year, healthy years equivalents, healthy days gained, episode-free day, Q-TWiST, Health Utilities Index, and years of healthy life.

In one example, health-adjusted life expectancy is measured by the health-adjusted life expectancy (HALE) index as described in Wilkins, R. and Adams, O B., Am J Public Health, 73:1073-1080 (1983). Health-adjusted life expectancy is an average of the quality-adjusted life years (QALY) for a given population and can be used to evaluate the therapeutic value of a medical intervention. Quality-adjusted life years is a health index that weighs each year of life on a scale from 1 to 0 (Weinstein M C and Stason W B, N Engl J Med, 296:716-721 (1977)). Perfect health is rated as 1, death is rated as 0, and disability and pain are rated based on severity. QALY is determined by multiplying the number of years at each health status.

QALY is determined by surveying individual subjects regarding their health status. Several surveys are used by clinicians including the Health Utilities Index (Mark I, Mark II, and Mark III), Short Form Health Status Survey (SF-36), Nottingham Health Profile, Sickness Impact Profile, EuroQoL (EQ-5D), and the Quality of Well-Being Scale. In general, all of these surveys attempt to measure the physical and emotional well-being of subjects.

In one embodiment, a method of treating multiple sclerosis is provided that reduces the severity or delays the onset of symptoms of the disease. Common symptoms of MS include visual symptoms such as optic neuritis, diplopis, and ocular dysmetria; motor symptoms including paresis, paralysis, spasms, and dysfunctional reflexes; sensory symptoms such as paraesthesia, neuralgia, and proprioceptive dysfunction; coordination symptoms such as ataxia, vertigo, dysmetria, and dystonia; cognitive symptoms including depression, cognitive dysfunction, and dementia; as well as other symptoms including fatigue, sleeping disorders, and erectile dysfunction. There are several methods clinicians use to measure the severity and progress of MS including the Scripps Neurologic Rating Scale (SNRS), Krupp Fatigue Severity Scale (FSS), Incapacity Status Scale (ISS), Functional Independence Measure (FIM), Ambulation Index (AI), Cambridge Multiple Sclerosis Basic Score (CAMBS), Functional Assessment of Multiple Sclerosis (FAMS), Profile of Mood States (POMS), Sickness Impact Profile (SIP), Kurtzke Expanded Disability Status Scale (EDSS), as well as MRI scans to observe MS lesions. A statistically significant change in score of one or more of these or other clinically accepted standard of disease severity following treatment is indicative of effective treatment.

In one embodiment, the agent used in the methods of treatment reduces TIM-3 activity. In one embodiment, the agent reduces TIM-3 signaling. In one embodiment, the agent used reduces TNF-α secretion by APCs, in particular CD11b⁺ microglia. In one embodiment, the agent used reduces TNF-α secretion by DCs.

In one embodiment, TIM-3 activity is decreased via a reduction in TIM-3 expression, such as, for example, as a result of treatment with TIM-3 RNAi or RNAi to a TIM-3 ligand. In one embodiment, TIM-3 activity is decreased via blocking the interaction of TIM-3 with one or more TIM-3 ligands. In one embodiment, the agent that blocks the interaction of TIM-3 with one or more TIM-3 ligands is an antagonist antibody or a polypeptide as disclosed herein. In one embodiment, the TIM-3 ligand is not galectin-9. In one embodiment, the agent is a carbohydrate or a pectin. In one embodiment, the carbohydrate is lactose or beta-galactoside.

Agents that inhibit TIM-3 can be tested for their ability to treat multiple sclerosis by any number of means appreciated by those skilled in the art. Experimental allergic encephalomyelitis (EAE) is a mouse model for human MS (old et al., Mol. Med. Today, 6:88-91, 2000; Anderton et al., Immunol. Rev., 169:123-137, 1999). MS is induced by immunizing mice with a peptide of the myelin protein MOG (myelin oligodendrocyte glycoprotein). This protein is present on the outside of the myelin sheath and acts as a protective layer for myelin. Mice are immunized subcutaneously with MOG peptide (MOG35-55) emulsified in RIBI adjuvant on day 0. Mice are then injected intravenously with pertussis toxin (PT) on day 2. The mice start showing symptoms of paralysis starting with a limp tail, wobbly motion, followed by hind limb and forelimb paralysis, which are scored according to several different parameters that measure the timing, extent and severity of disease. Delay in onset of disease indicates that the agent is modifying the disease process in mice. Decrease in incidence indicates that the agent is having an effect on the number of mice that are getting sick. Decrease in clinical score indicates that the agent has an effect on the severity of disease.

The effect of the agents in human subjects can be monitored by observation of the incidence and severity of common symptoms of multiple sclerosis, such as vertigo, visual dysfunction, fatigue, and cognitive slowing. Both the Kurtzke Extended Disability Status Score (EDSS) and the MS Functional composite (MSFC) score can be employed for monitoring disability. As noted above, a statistically significant improvement in either of these scores indicates effective therapy.

The preferred amount of the compounds of the invention is a therapeutically effective amount thereof which is also medically acceptable. Actual dosage levels of the pharmaceutical compositions of the present invention may be varied so as to obtain an amount which is effective to achieve the desired therapeutic response for a particular patient, pharmaceutical composition, and mode of administration, without being toxic to the patient. The selected dosage level and frequency of administration will depend upon a variety of factors including the route of administration, the time of administration, the duration of the treatment, other drugs, compounds and/or materials used in combination with the compounds of the invention, the age, sex, weight, condition, general health and prior medical history of the patient being treated, and the like factors well known in the medical arts. A physician having ordinary skill in the art can readily determine and prescribe the therapeutically effective amount of the pharmaceutical composition required.

Where relevant, effective amounts of a vaccine can be determined, for example, by measuring increases in the immune response, for example, by the presence of higher titers of antibody, the presence of higher affinity antibodies, the presence of a desired population of immune cells such as memory cells to a particular antigen, or the presence of particular antigen specific cytotoxic T cells. Effective amounts also can be measured by a reduction in microbial load in the case of an infection or in the size or progression of a tumor in the case of cancer. An effective amount also can be reflected in a reduction in the symptoms experienced by a particular subject being treated.

For the treatment of inflammatory disorders of the CNS, dosage can be adjusted appropriately to achieve desired drug levels, locally or systemically. Generally, doses of compounds will be from about 0.001 mg/kg per dose to 1000 mg/kg per dose. Administration of these levels can be, e.g., daily, up to several times per day, or at longer intervals, depending on the formulation administered. Where stabilized or slow-release formulations are administered, dosages can be given, e.g., daily, every other day, every 2 days, every 3 days, etc., weekly, bi-weekly, or even monthly. It is generally expected that doses in the range of about 0.1 to 50 mg/kg per day will be effective. In the event that the response in a subject is insufficient at such doses, even higher doses (or effective higher doses by a different, more localized delivery route) may be employed to the extent that patient tolerance permits.

A variety of administration routes are available. The particular mode selected will depend of course, upon the particular drug selected, the severity of the disease state being treated and the dosage required for therapeutic efficacy. The methods of this invention, generally speaking, may be practiced using any mode of administration that is medically acceptable, meaning any mode that produces effective levels of the active compounds without causing clinically unacceptable adverse effects. Such modes of administration include oral, rectal, sublingual, topical, nasal, transdermal or parenteral routes. The term “parenteral” includes subcutaneous, intravenous, intramuscular, or infusion. Oral and intravenous routes are preferred.

### VI. Methods of Treating Tumors

The invention further provides novel methods of treating tumors, of inhibiting tumor/cancer growth, prolonging the life of a subject afflicted with a tumor, or of reducing one or more symptoms associated with tumors. One aspect of the invention provides a method of treating a tumor/cancer in a subject in need of such treatment, the method comprising administering to the subject a therapeutically effective amount of an agent that increases TIM-3 activity in the subject.

One aspect of the invention provides a method of prolonging life expectancy in a subject afflicted with a tumor; the method comprising administering to a subject a therapeutically effective amount of an agent that increases TIM-3 activity in the subject. In one embodiment, the agent that increases TIM-3 activity prolongs the life expectancy of a subject by at least one day, at least one week, at least two weeks, at least three weeks, at least one month, at least two months, at least three months, at least 6 months, or at least one year.

One aspect of the invention provides a method of prolonging health-adjusted life expectancy in a subject afflicted with a tumor; the method comprising administering to a subject a therapeutically effective amount of an agent that decreases TIM-3 activity in the subject. In one embodiment, the agent that increases TIM-3 activity prolongs the health-adjusted life expectancy of a subject by at least one day, at least one week, at least two weeks, at least three weeks, at least one month, at least two months, at least three months, at least 6 months, or at least one year.

One aspect of the invention provides a method to treat subjects at risk of developing cancer. A subject at risk of developing a cancer is one who has a higher than normal probability of developing cancer, such as relative to the general population or to a population matched to one of more of risk factors such as age, gender, race, and family history. These subjects include, for instance, subjects having a genetic abnormality that has been demonstrated to be associated with a higher likelihood of developing a cancer, subjects having a familial disposition to cancer, subjects exposed to cancer causing agents (i.e., carcinogens) such as tobacco, asbestos, or other chemical toxins, and subjects previously treated for cancer and in apparent remission. One aspect of the invention provides a method of treating a subject at risk of developing a cancer, the method comprising administering to the subject a therapeutically effective amount of an agent that increases TIM-3 activity in the subject.

The methods of treating tumors are based in part on the surprising discovery that microglia obtained from glioblastoma multiforme (GBM) express TIM-3 at reduced levels compared to control tissue. It has been reported that microglia distributed throughout and around glial tumors are functionally impaired. In particular, MHC-II induction in response to activators, such as CpG oligodeoxynucleotide, interferon-gamma, and IFN-gamma/LPS, is significantly reduced in microglia obtained from brain tumors (Schartner, J. M. et al. Glia 51, 279-85 (2005)). This aspect of the invention provides a treatment for tumors based on increasing TIM-3 activity.

“Tumor”, as used herein, refers to all neoplastic cell growth and proliferation, whether malignant or benign, and all pre-cancerous and cancerous cells and tissues. In one embodiment, the tumor is a tumor of the central nervous system.

There are two types of cancers, benign and malignant. Nearly all benign cancers are encapsulated and are noninvasive; in contrast, malignant cancers are almost never encapsulated but invade adjacent tissue by infiltrative destructive growth. This infiltrative growth can be followed by cancer cells implanting at sites discontinuous with the original cancer. The agents of the invention can be used to treat cancers in humans, including but not limited to: sarcomas, carcinomas, fibromas, leukemias, lymphomas, melanomas, myelomas, neuroblastomas, rhabdomyosarcomas, retinoblastomas, and gliomas, as well as each of the other cancers described herein.

Cancers that migrate from their original location and seed vital organs (thereby giving rise to metastatic lesions) can eventually lead to the death of the subject through the functional deterioration of the affected organs. A metastasis is a region of cancer cells, distinct from the primary cancer location resulting from the dissemination of cancer cells from the primary cancer to other parts of the body. Thus, subjects with metastatic cancers can also be treated according to the invention. In some embodiments, the metastatic cancers are of epithelial origin. Carcinomas may metastasize to bone, as has been observed with breast cancer, and liver, as is sometimes the case with colon cancer. The methods of the invention are intended to treat metastatic cancers regardless of the site of the metastasis and/or the site of the primary cancer.

In one embodiment, the application provides a method of treating brain tumors including meningiomas; gliomas including ependymomas, oligodendrogliomas, and all types of astrcytomas (low grade, anaplastic, and glioblastoma multiforme or simply glioblastoma); medullablastomas, gangliogliomas, schwannomas, chordomas; and brain tumors primarily of children including primitive neuroectodermal tumors. Both primary brain tumors (i.e., arising in the brain) and secondary or metastatic brain tumors can be treated by the methods of the invention.

In one embodiment, the tumor is selected from astrocytomas, oligodendrogliomas, ependymoma, mixed gliomas, oligoastrocytomas, gangliogliomas, or glioblastoma multiforme, or neurofibromatosis.

In one embodiment, the tumor is an astrocytoma. Astrocytomas are glioma tumors that arise from brain cells called astrocytes or their precursors. Astrocytes are cells in the central nervous system that support neuronal function. Astrocytomas can be graded by histological features that signify increasing malignancy into astrocytoma, anaplastic astrocytoma, or glioblastoma multiforme. Anaplastic astrocytoma and glioblastoma multiforme are considered high-grade gliomas while the astrocytoma is considered to be a low-grade glioma. High-grade tumors grow rapidly and can easily infiltrate and spread through the brain. Low-grade astrocytomas can also infiltrate the brain but are usually more localized and grow slowly over a long period of time. High-grade tumors are much more aggressive and require very intense therapy. The majority of astrocytic tumors in children are low-grade, whereas the majority in adults are high-grade. Astrocytomas can occur anywhere in the brain and spinal cord, however the majority are located in the cerebral hemispheres.

In one embodiment, the tumor is an anaplastic glioma. The anaplastic gliomas are intermediate grade infiltrative gliomas—classified between low (localized, slow growing) and glioblastoma multiforme (rapidly growing and highly invasive). Anaplastic astrocytomas (AA) are tumors that arise from brain cells called astrocytes and/or their precursors.

In one embodiment, the tumor is a glioblastoma multiforme. Glioblastoma, also known as glioblastoma multiforme, is the glioma with the highest grade of malignancy, WHO grade IV (Kleihues and Cavenee, 2000). It represents 15% to 23% of intracranial tumors and about 50%-60% of astrocytomas. Most examples are generally considered to arise from astrocytes because glial fibrillary acidic protein can be identified in the cell cytoplasm. Some examples, however, apparently arise from other glial lineages, such as oligodendrocytes. Glioblastoma is the most frequently occurring astrocytoma. Autopsy and serial biopsy studies have shown that some astrocytomas progress through the grades of malignancy with transformation from low-grade to anaplastic astrocytoma to glioblastoma (Muller et al., 1977). But, because some examples of glioblastoma appear to arise rapidly in otherwise normal patients and are recognized when they are small, it is thought that this variety of glioblastoma can also arise directly from malignant transformation of astrocyte precursor cells without passing through the lower grades of malignancy (Kleihues and Ohgaki, 1997; 1999). The TIM-3 enhancing agents devised herein may be used in combination with additional methods of tumor treatment.

U.S. Patent Pub. Nos: 2007-0032453 and 2006-0281720 disclose additional methods of treating glial tumors that may be used in combination with the TIM-3 enhancing agents described herein.

Cancers include, but are not limited to, basal cell carcinoma, biliary tract cancer; bladder cancer; bone cancer; brain and CNS cancer; breast cancer; cervical cancer; choriocarcinoma; colon and rectum cancer; connective tissue cancer; cancer of the digestive system; endometrial cancer; esophageal cancer; eye cancer; cancer of the head and neck; gastric cancer; germ cell tumors; intra-epithelial neoplasm; Kaposi's sarcoma; kidney cancer; larynx cancer; leukemia (e.g., acute myeloid leukemia, acute lymphoid leukemia, chronic myeloid leukemia and chronic lymphoid leukemia); liver cancer; lung cancer (e.g. small cell and non-small cell); lymphoma including Hodgkin's and Non-Hodgkin's lymphoma; melanoma; myeloma; neuroblastoma; oral cavity cancer (e.g., lip, tongue, mouth, and pharynx); ovarian cancer; pancreatic cancer; prostate cancer; renal cell cancer; retinoblastoma; rhabdomyosarcoma; rectal cancer; renal cancer; cancer of the respiratory system; sarcoma; skin cancer; stomach cancer; stromal tumors; testicular cancer; thyroid cancer; uterine cancer; cancer of the urinary system, as well as other carcinomas and sarcomas.

The cancers to be treated may be refractory cancers. A refractory cancer as used herein is a cancer that is resistant to the ordinary standard of care prescribed. These cancers may appear initially responsive to a treatment (and then recur), or they may be completely non-responsive to the treatment. The ordinary standard of care will vary depending upon the cancer type, and the degree of progression in the subject. It may be a chemotherapy, surgery, or radiation, or a combination thereof. Those of ordinary skill in the art are aware of such standards of care. Subjects being treated according to the invention for a refractory cancer therefore may have already been exposed to another treatment for their cancer. Alternatively, if the cancer is likely to be refractory (e.g., given an analysis of the cancer cells or history of the subject), then the subject may not have already been exposed to another treatment. Examples of refractory cancers include but are not limited to leukemias, melanomas, renal cell carcinomas, colon cancer, liver cancers, pancreatic cancer, and lung cancer.

The compositions and methods of the invention in certain instances may be useful for replacing existing surgical procedures or drug therapies, although in most instances the present invention is useful in improving the efficacy of existing therapies for treating such conditions. Accordingly combination therapy may be used to treat the subjects that are undergoing or that will undergo a treatment for, inter alia, infectious disease or cancer. For example, the agents of the present invention can be administered in conjunction with anti-microbial agents or anti-proliferative agents. The agents of the invention also can be administered in conjunction with other immunotherapies, such as with antigens, adjuvants, immunomodulators, or passive immune therapy with antibodies. The agents of the invention also can be administered in conjunction with nondrug treatments, such as surgery, radiation therapy or chemotherapy. The other therapy may be administered before, concurrent with, or after treatment with the agents of the invention. There may also be a delay of several hours, days and in some instances weeks between the administration of the different treatments, such that the agents of the invention may be administered before or after the other treatment.

In one embodiment, administration of the agent to the cancer subject increases TIM-3 activity in APCs. In one embodiment, the APCs are monocytes or CD11b⁺ microglia cells. In another embodiment the cells are in the central nervous system.

In one embodiment, an agent is administered to a subject that increases TIM-3 signaling. In one embodiment, the agent used increases TNF-α secretion by APCs. In one embodiment, the APCs are monocytes, DCs, or CD11b⁺ microglia cells. In one embodiment, the agent increases phosphorylation of the intracellular domain of TIM-3. In one embodiment, an agent is administered to a subject that increases TIM-3 signaling with the proviso that the agent is not galectin-9.

In one embodiment, the agent is an antibody or antibody fragment, wherein the antibody acts as a TIM-3 agonist. In one embodiment, the agent is a TIM-3 ligand or a compound that mimics the effect of a TIM-3 ligand. In one embodiment the TIM-3 ligand comprises a galectin-9 polypeptide or is at least 90%, at least 95%, or at least 99% identical to a galectin-9 polypeptide. In another embodiment, the agent is a TIM-3 ligand with the proviso that the ligand is not galectin-9 and/or an anti-galectin-9 antibody.

To test agents of the invention for their ability to treat tumors in vivo, tumors can be explanted into nude mice (i.e., athymic mice). Various xenograft models are known in the art. After the tumors are established in mice, the agents that increase TIM-3 activity are administered to the mice in order to test for whether the agents can diminish the tumors or prolong median survival of the animals.

In one embodiment, the agent that increases TIM-3 activity is administered together in combination with (i.e., before, during or after) other anti-cancer therapy. For example, the agent may be administered together with any one or more of the chemotherapeutic drugs known to those of skill in the art of oncology, for example alkylating agents such as carmustine, chlorambucil, cisplatin, carboplatin, oxiplatin, procarbazine, and cyclophosphamide; antimetabolites such as fluorouracil, floxuridine, fludarabine, gemcitabine, methotrexate and hydroxyurea; natural products including plant alkaloids and antibiotics such as bleomycin, doxorubicin, daunorubicin, idarubicin, etoposide, mitomycin, mitoxantrone, vinblastine, vincristine, and Taxol (paclitaxel) or related compounds such as Taxotere™; agents specifically approved for brain tumors including temozolomide and Gliadel™; wafer containing carmustine; and other drugs including irinotecan and Gleevec™; and all approved and experimental anti-cancer agents listed in WO 2005/017107 A2 (which is herein incorporated by reference). The agent can be administered in combination with 1, 2, 3 or more of these agents, e.g., in a standard chemotherapeutic regimen. Other agents with which an agent that increases TIM-3 activity can be administered include biologics such as monoclonal antibodies, including Herceptin™ against the HER2 antigen, Avastin™ against VEGF, antibodies to the EGF receptor such as Erbitux™, or an anti-FGF mAb, as well as small molecule anti-angiogenic or EGF receptor antagonist drugs such as Iressa™ and Tarceva™. In addition, the agent can be administered together with any form of radiation therapy including external beam radiation, intensity modulated radiation therapy (IMRT) and any form of radiosurgery including Gamma Knife, Cyberknife, Linac, and interstitial radiation (e.g. implanted radioactive seeds, GliaSite balloon). In one embodiment, the agent is administered during a surgical procedure, such as, for example, during the removal of a tumor or a tumor biopsy.

The agents of the invention also are used with nondrug treatments for cancer, such as with surgical procedures to remove the cancer mass, chemotherapy or radiation therapy. The nondrug therapy may be administered before, concurrent with, or after treatment with the agents of the invention. There may also be a delay of several hours, days and in some instances weeks between the administration of the different treatments, such that the agents of the invention may be administered before or after the other treatment.

Surgical methods for treating cancer include intra-abdominal surgeries such as right or left hemicolectomy, sigmoid, subtotal or total colectomy and gastrectomy, radical or partial mastectomy, prostatectomy and hysterectomy. In one embodiment, the agents that increase TIM-3 activity are administered locally to an area of cancerous mass after or during surgical removal of a tumor.

The invention in one embodiment contemplates the use of agents of the invention in cancer subjects prior to surgery, radiation or chemotherapy in order to create memory immune cells to the cancer antigen. In this way, memory cells of the immune system can be primed with cancer antigens and thereby provide immune surveillance in the long term. Immune cells so primed can invade a tumor site and effectively clear any remaining tumor debris following the other treatment.

The agents of the invention can be used with cancer antigens. A cancer antigen as used herein is a compound differentially associated with a cancer, preferably at the cell surface of a cancer cell (or even at the surface of the neovasculature), that is capable of invoking an immune response. The antigen invokes an immune response when it is presented (in a digested form) on the surface of an antigen presenting cell in the context of an MHC molecule. Cancer antigens can be prepared from cancer cells either by preparing crude extracts of cancer cells, for example, as described in Cohen, et al., 1994, Cancer Research, 54:1055, by partially purifying the antigens, by recombinant technology, or by de novo synthesis of known antigens. Cancer antigens include but are not limited to antigens that are recombinantly expressed, an immunogenic portion of, or a whole tumor or cancer. Such antigens can be isolated or prepared recombinantly or by any other means known in the art.

A cancer antigen encompasses antigens that are differentially expressed between cancer and normal cells. Due to this differential expression, these antigens can be targeted in anti-tumor therapies. Cancer antigens may be expressed in a regulated manner in normal cells. For example, they may be expressed only at certain stages of differentiation or at certain points in development of the organism or cell. Some are temporally expressed as embryonic and fetal antigens. Still others are never expressed in normal cells, or their expression in such cells is so low as to be undetectable.

Other cancer antigens are encoded by mutant cellular genes, such as oncogenes (e.g., activated ras oncogene), suppressor genes (e.g., mutant p53), fusion proteins resulting from internal deletions or chromosomal translocations. Still other cancer antigens can be encoded by viral genes such as those carried on RNA and DNA tumor viruses.

Examples of cancer antigens include HER-2 (p185), CD20, CD33, GD3 ganglioside, GD2 ganglioside, carcinoembryonic antigen (CEA), CD22, milk mucin core protein, TAG-72, Lewis A antigen, ovarian associated antigens such as OV-TL3 and MOv18, high Mr melanoma antigens recognized by antibody 9.2.27, HMFG-2, SM-3, B72.3, PR5C5, PR4D2, and the like. Other cancer antigens are described in U.S. Pat. No. 5,776,427.

Further examples include MAGE, MART-1/Melan-A, gp100, Dipeptidyl peptidase IV (DPPIV), adenosine deaminase-binding protein (ADAbp), FAP, cyclophilin b, Colorectal associated antigen (CRC)-C017-1A/GA733, Carcinoembryonic Antigen (CEA) and its immunogenic epitopes CAP-1 and CAP-2, etv6, aml1, Prostate Specific Antigen (PSA) and its immunogenic epitopes PSA-1, PSA-2, and PSA-3, prostate-specific membrane antigen (PSMA), T-cell receptor/CD3-zeta chain, MAGE-family of tumor antigens (e.g., MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-05), GAGE-family of tumor antigens (e.g., GAGE-1, GAGE-2, GAGE-3, GAGE-4, GAGE-5, GAGE-6, GAGE-7, GAGE-8, GAGE-9), BAGE, RAGE, LAGE-1, NAG, GnT-V, MUM-1, CDK4, tyrosinase, p53, MUC family, HER2/neu, p21 ras, RCAS1, α-fetoprotein, E-cadherin, α-catenin, β-catenin and γ-catenin, p120ctn, gp100^{Pmell17}, PRAME, NY-ESO-1, cdc27, adenomatous polyposis coli protein (APC), fodrin, Connexin 37, Ig-idiotype, p15, gp75, GM2 and GD2 gangliosides, viral products such as human papilloma virus proteins, Smad family of tumor antigens, Imp-1, P1A, EBV-encoded nuclear antigen (EBNA)-1, brain glycogen phosphorylase, SSX-1, SSX-2 (HOM-MEL-40), SSX-1, SSX-4, SSX-5, SCP-1 and CT-7, CD20 and c-erbB-2.

Cancer or tumor antigens can also be classified according to the cancer or tumor they are associated with (i.e., expressed by). Cancers or tumors associated with tumor antigens include acute lymphoblastic leukemia (etv6; aml1; cyclophilin b), B cell lymphoma (Ig-idiotype); Burkitt's (Non-Hodgkin's) lymphoma (CD20); glioma (E-cadherin; α-catenin; β-catenin; γ-catenin; p120ctn), bladder cancer (p21ras), biliary cancer (p21ras), breast cancer (MUC family; HER2/neu; c-erbB-2), cervical carcinoma (p53; p21ras), colon carcinoma (p21ras; HER2/neu; c-erbB-2; MUC family), colorectal cancer (Colorectal associated antigen (CRC)-C017-1A/GA733; APC), choriocarcinoma (CEA), epithelial cell-cancer (cyclophilin b), gastric cancer (HER2/neu; c-erbB-2; ga733 glycoprotein), hepatocellular cancer (α-fetoprotein), Hodgkin's lymphoma (lmp-1; EBNA-1), lung cancer (CEA; MAGE-3; NY-ESO-1), lymphoid cell-derived leukemia (cyclophilin b), melanoma (p15 protein, gp75, oncofetal antigen, GM2 and GD2 gangliosides), myeloma (MUC family; p21ras), non-small cell lung carcinoma (HER2/neu; c-erbB-2), nasopharyngeal cancer (Imp-1; EBNA-1), ovarian cancer (MUC family; HER2/neu; c-erbB-2), prostate cancer (Prostate Specific Antigen (PSA) and its immunogenic epitopes PSA-1, PSA-2, and PSA-3; PSMA; HER2/neu; c-erbB-2), pancreatic cancer (p21ras; MUC family; HER2/neu; c-erbB-2; ga733 glycoprotein), renal (HER2/neu; c-erbB-2), squamous cell cancers of cervix and esophagus (viral products such as human papilloma virus proteins and non-infectious particles), testicular cancer (NY-ESO-1), T cell leukemia (HTLV-1 epitopes), and melanoma (Melan-A/MART-1; cdc27; MAGE-3; p21ras; gp100^{Pmell17}). In some preferred embodiments, the cancer antigen is VEGF, Anti-idiotypic mAb (GD3 ganglioside mimic), CD20, CD52, Anti-idiotypic mAb (CEA mimic), ERBB2, EGFR, CD22, ERBB2 X CD65 (fcγRI), EpCam, PEM and CD33.

### VII. Stimulating an Immune Response

Also disclosed herein are vaccine compositions comprising an antigen and as an adjuvant, an agent that increases TIM-3 activity. The disclosed compositions may also be useful for treating viral infections, enhancing tumor immunity, enhancing vaccination efficacy or in ameliorating immune suppression. Agents that increase TIM-3 activity can be useful for enhancing the efficacy of vaccines, such as to treat infectious agents and/or cancer.

In one embodiment, the agent that increases TIM-3 activity increases TNF-α secretion by APCs. In one embodiment, the agent increases phosphorylation of the intracellular domain of TIM-3.

In one embodiment, the agent is an antibody or antibody fragment, wherein the antibody acts as a TIM-3 agonist. In one embodiment, the agent is a TIM-3 ligand or a compound that mimics the effect of a TIM-3 ligand. In one embodiment the TIM-3 ligand comprises a galectin-9 polypeptide or is at least 90%, at least 95%, or at least 99% identical to a galectin-9 polypeptide. In another embodiment, the agent is a TIM-3 ligand with the proviso that the ligand is not galectin-9.

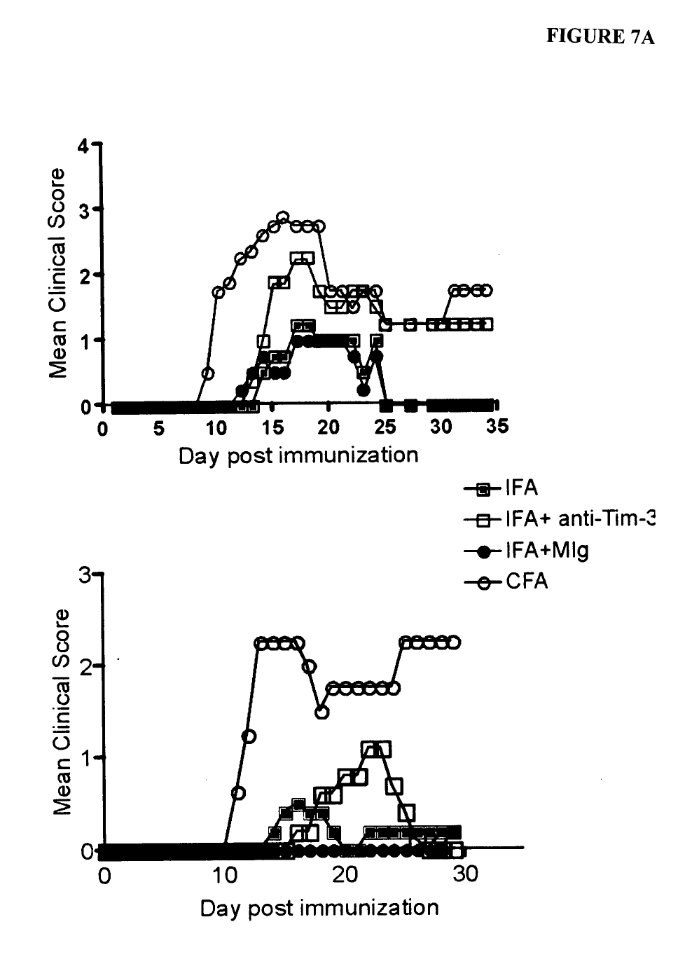

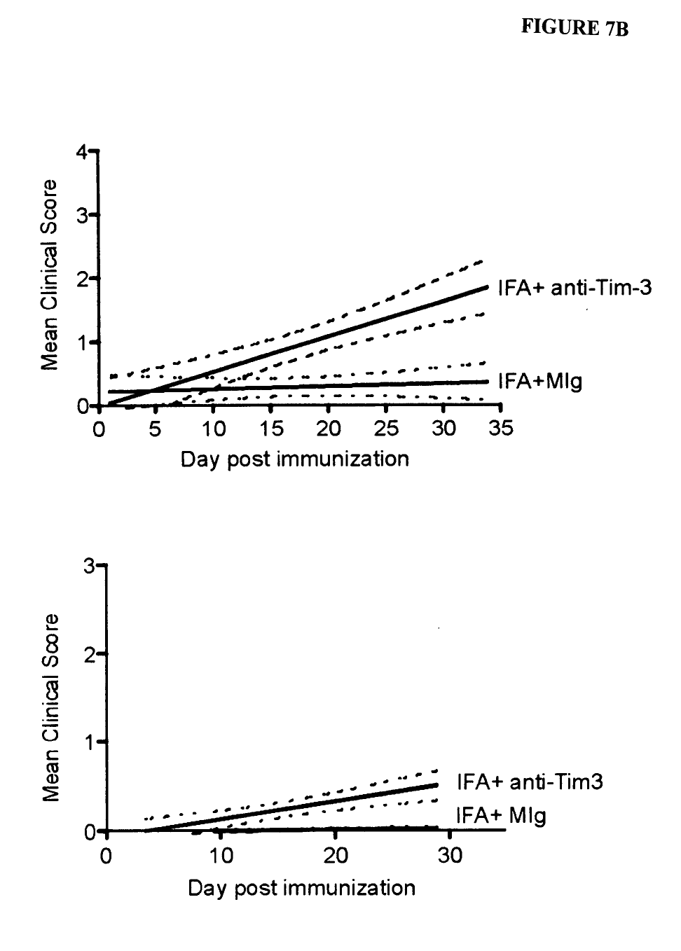

The methods for enhancing immune responses are based in part on the surprising discovery that TIM-3 expression on dendritic cells could be used to promote inflammatory Th1 responses in vivo. It was observed that mice immunized with IFA (incomplete Freund's adjuvant) containing agonistic anti-TIM-3 antibody developed more severe disease than mice immunized with IFA and control antibody (see e.g., FIG. 7).

In one embodiment, a vaccine composition comprises an antigen, and as an adjuvant, an agent that increases TIM-3 activity, and further comprises a TLR ligand. At least 10 human and 12 murine TLRs (Toll-like receptors) have been described (see, e.g., Takeda et. al., Annu. Rev. Immunol. 21:335, 2003; see also below). One or more ligands that interact with and subsequently activate certain TLRs have been identified. In one embodiment, the ligand binds to one or more of TLR1, TLR2, TLR3, TLR4, TLR5, TLR6, TLR7 or TLR9. Certain outer membrane proteins of Neisseria meningitidis, for example OMP 2 (also referred to as Por B), interact with TLR2, while LPS of most but not all Gram-negative bacteria interacts with TLR4. Examples of other TLR ligands include soluble factors (e.g., Neisseria meningitidis), tri-acyl lipopeptides (bacteria, mycobacteria), lipoproteins and lipopeptides, porins (Neisseria), atypical LPS (e.g., Leptospira interrogans, P. gingivalis), peptidoglycan (Gram-positive bacteria), lipoteichoic acid (Gram-positive bacteria), HSP70 (host), glycolipids (e.g., Treponema maltophilum), double-stranded RNA (e.g., viral), LPS (Gram-negative bacteria), taxol (plant), HSP60 (host), HSP70 (host), HSP60 (Chlamydia pneumoniae), fibrinogen (host), flagellin (bacteria), di-acyl lipopeptides (mycoplasma), imidazoquinoline (synthetic compounds), loxoribine (synthetic compounds), bropirimine (synthetic compounds), and CpG DNA (bacteria). In one embodiment, the agent may be formulated into a TLR ligand:liposome complex as described in US application 2005/0013812. A wide range of TLR ligands are available commercially, e.g., from Invivogen (San Diego, Calif.).

TLRs are type I transmembrane proteins, each characterized by an extracellular leucine-rich domain and a cytoplasmic tail that contains a conserved region referred to as the “Toll/IL-1 receptor” (TIR) domain. TLRs are generally expressed in tissues involved in immune function, e.g., spleen and peripheral blood leukocytes, as well as those exposed to the external environment, e.g., the gastrointestinal tract and the lung. The expression patterns of TLRs varies among tissues and cell types. TLRs are located on the plasma membrane, except for TLR3, TLR7 and TLR9, which are intracellular.

At least ten human and twelve murine TLRs have been characterized, TLR1 to TLR10 in humans, and TLR1 to TLR9, TLR11, TLR12 (aka TLR11) and TLR13 in mice, the homolog of TLR10 being a pseudogene. TLR2 is essential for the recognition of a variety of pathogen-associated molecular patterns from Gram-positive bacteria, including bacterial lipoproteins, lipomannans and lipoteichoic acids. TLR3 is implicated in recognition of virus-derived double-stranded RNA. TLR4 is predominantly activated by lipopolysaccharide. TLR5 detects bacterial flagellin and TLR9 is required for response to unmethylated CpG DNA. TLR7 and TLR8 have been reported to recognize small synthetic antiviral molecules (Jurk et al., 2002, Nat. Immunol., 3:499), and recently single-stranded RNA was reported to be their natural ligand (Heil et al., 2004, Science, 303:1526-9). TLR11 has been reported to recognize uropathogenic E. coli and a profilin-like protein from Toxoplasma gondii.

TLRs can heterodimerize with one another, resulting in a broadened range of specificities. For example, dimers of TLR2 and TLR6 are required for responses to diacylated lipoproteins while TLR2 and TLR1 interact to recognize triacylated lipoproteins. Specificities of the TLRs are also influenced by various adapter and accessory molecules, such as MD-2 and CD14 that form a complex with TLR4 in response to LPS.

As used herein, the term “TLR signaling activity” refers to one or more signaling activities of a TLR that is induced upon ligand binding by the receptor. TLR signaling occurs through at least two distinct pathways: a MyD88-dependent pathway that leads to the production of inflammatory cytokines, and a MyD88-independent pathway associated with the stimulation of IFN-β and the maturation of dendritic cells. The MyD88-dependent pathway is common to all TLRs. Upon activation by microbial antigens, TLRs induce the recruitment of MyD88 via its TIR domain which activates IRAK-1 by phosphorylation. IRAK-1 then leaves the MyD88-TLR complex and associates temporarily with TRAF6. This association elicits downstream signaling, leading to the activation of NF-κB which in turn induces the production of pro-inflammatory cytokines such as TNF-α, IL-1 and IL-12. Activity of a TLR can thus be measured by measurement of any such downstream signalling activity, including, but not limited to reporters sensitive to the activities of intermediaries in the pathways, or, e.g., reporters responsive to NF-kB or AP-1, which are activated by TLR signalling.

In one embodiment, the vaccine composition comprises an antigen and as an adjuvant, an agent that increases TIM-3 activity, and further comprises one or more additional adjuvants, including, for example, a non-nucleic acid adjuvant, a nucleic acid adjuvant, a non-nucleic acid mucosal adjuvant, or an immune stimulating adjuvant.

A “nucleic acid adjuvant” is an adjuvant that is a nucleic acid or analog thereof. Examples include immunostimulatory nucleic acid molecules such as those containing CpG dinucleotides, as described in U.S. Pat. No. 6,194,388, issued Feb. 27, 2001, U.S. Pat. No. 6,207,646, issued Mar. 27, 2001, and U.S. Pat. No. 6,239,116, issued May 29, 2001.

A “non-nucleic acid adjuvant” is any molecule or compound other than immunostimulatory nucleic acids, which can stimulate the humoral and/or cellular immune response. Non-nucleic acid adjuvants include, for instance, adjuvants that create a depot effect, immune-stimulating adjuvants, adjuvants that create a depot effect and stimulate the immune system and mucosal adjuvants.

An immune stimulating adjuvant is an adjuvant that causes activation of a cell of the immune system. It may, for instance, cause an immune cell to produce and secrete cytokines. This class of adjuvants includes but is not limited to saponins purified from the bark of the Q. saponaria tree, such as QS21 (a glycolipid that elutes in the 21^{st} peak with HPLC fractionation; Antigenics, Inc., Waltham, Mass.); poly[di(carboxylatophenoxy) phosphazene (PCPP polymer; Virus Research Institute, USA); derivatives of lipopolysaccharides such as monophosphoryl lipid A (MPL; Ribi ImmunoChem Research, Inc., Hamilton, Mont.), muramyl dipeptide (MDP; Ribi) and threonyl-muramyl dipeptide (t-MDP; Ribi); OM-174 (a glucosamine disaccharide related to lipid A; OM Pharma SA, Meyrin, Switzerland); and Leishmania elongation factor (a purified Leishmania protein; Corixa Corporation, Seattle, Wash.).

A non-nucleic acid mucosal adjuvant is an adjuvant other than an immunostimulatory nucleic acid that is capable of inducing a mucosal immune response in a subject when administered to a mucosal surface in conjunction with an antigen. Mucosal adjuvants include but are not limited to Bacterial toxins: e.g., Cholera toxin (CT), CT derivatives including but not limited to CT B subunit (CTB) (Wu et al., 1998, Tochikubo et al., 1998); CTD53 (Val to Asp) (Fontana et al., 1995); CTK97 (Val to Lys) (Fontana et al., 1995); CTK104 (Tyr to Lys) (Fontana et al., 1995); CTD53/K63 (Val to Asp, Ser to Lys) (Fontana et al., 1995); CTH54 (Arg to His) (Fontana et al., 1995); CTN107 (His to Asn) (Fontana et al., 1995); CTE114 (Ser to Glu) (Fontana et al., 1995); CTE112K (Glu to Lys) (Yamamoto et al., 1997a); CTS61F (Ser to Phe) (Yamamoto et al., 1997a, 1997b); CTS106 (Pro to Lys) (Douce et al., 1997, Fontana et al., 1995); and CTK63 (Ser to Lys) (Douce et al., 1997, Fontana et al., 1995), Zonula occludens toxin, zot, Escherichia coli heat-labile enterotoxin, Labile Toxin (LT), LT derivatives including but not limited to LT B subunit (LTB) (Verweij et al., 1998); LT7K (Arg to Lys) (Komase et al., 1998, Douce et al., 1995); LT61F (Ser to Phe) (Komase et al., 1998); LT112K (Glu to Lys) (Komase et al., 1998); LT118E (Gly to Glu) (Komase et al., 1998); LT146E (Arg to Glu) (Komase et al., 1998); LT192G (Arg to Gly) (Komase et al., 1998); LTK63 (Ser to Lys) (Marchetti et al., 1998, Douce et al., 1997, 1998, Di Tommaso et al., 1996); and LTR72 (Ala to Arg) (Giuliani et al., 1998), Pertussis toxin, PT. (Lycke et al., 1992, Spangler B D, 1992, Freytag and Clemments, 1999, Roberts et al., 1995, Wilson et al., 1995) including PT-9K/129G (Roberts et al., 1995, Cropley et al., 1995); Toxin derivatives (see below) (Holmgren et al., 1993, Verweij et al., 1998, Rappuoli et al., 1995, Freytag and Clements, 1999); Lipid A derivatives (e.g., monophosphoryl lipid A, MPL) (Sasaki et al., 1998, Vancott et al., 1998; Muramyl Dipeptide (MDP) derivatives (Fukushima et al., 1996, Ogawa et al., 1989, Michalek et al., 1983, Morisaki et al., 1983); Bacterial outer membrane proteins (e.g., outer surface protein A (OspA) lipoprotein of Borrelia burgdorferi, outer membrane protine of Neisseria meningitidis) (Marinaro et al., 1999, Van de Verg et al., 1996); Oil-in-water emulsions (e.g., MF59) (Barchfield et al., 1999, Verschoor et al., 1999, O'Hagan, 1998); Aluminum salts (Isaka et al., 1998, 1999); and Saponins (e.g., QS21) Aquila Biopharmaceuticals, Inc., Worcester, Mass.) (Sasaki et al., 1998, MacNeal et al., 1998), ISCOMS, MF-59 (a squalene-in-water emulsion stabilized with Span 85 and Tween 80; Chiron Corporation, Emeryville, Calif.); the Seppic ISA series of Montanide adjuvants (e.g., Montanide ISA 720; AirLiquide, Paris, France); PROVAX (an oil-in-water emulsion containing a stabilizing detergent and a micelle-forming agent; IDEC Pharmaceuticals Corporation, San Diego, Calif.); Syntext Adjuvant Formulation (SAF; Syntex Chemicals, Inc., Boulder, Colo.); poly[di(carboxylatophenoxy)phosphazene (PCPP polymer; Virus Research Institute, USA) and Leishmania elongation factor (Corixa Corporation, Seattle, Wash.).

One aspect of the invention relates to methods for the treatment of subjects having or at risk of having a disease and/or in a state of immunosuppression with agents that increase TIM-3 activity. For example, the subjects may have or be at risk of developing an infectious disease. In another example, the subject may have or be at risk of developing a cancer. In another example, the subjects may have or may be at risk of developing an immune system suppression, such as from a genetic condition, radiation treatment, chemotherapy, or an infection, such as a chronic infection. Subjects with abnormally low CD4 cell counts are one example of immune suppressed subjects. In general, the number of functional CD4⁺-T cells that is within a normal range is known for various mammalian species. In human blood, e.g., the number of functional CD4⁺-T cells which is considered to be in a normal range is from about 600 to about 1500 CD4⁺-T cells/mm³ blood. An individual having a number of CD4⁺-T cells below the normal range, e.g., below about 600/mm³, may be considered “CD4⁺-deficient.”

Subjects may be exposed to myeloid, lymphoid or general immune suppressing conditions by the use of either immunosuppressant drugs such as cyclosporin or high dose chemotherapeutic compounds which affect dividing hematopoietic cells. Immunosuppression may also arise as a result of treatment modalities such as total body irradiation or conditioning regimens prior to bone marrow transplantation. Viral infection, particularly as in the case of infection with human immunodeficiency virus (HIV), may also immunosuppress an individual. In some embodiments, subjects are those which have not been exposed and are not anticipated to be exposed to the above-mentioned conditions. In other embodiments, the instant invention aims to treat subjects who may have been myelosuppressed or immunosuppressed (e.g., by exposure to one or more of the above conditions).

The invention thus involves treatment in some embodiments of individuals who are immunocompromised and in other embodiments who are not immunocompromised. Subjects who are not immunocompromised are those that have blood cell counts in the normal range. Subjects who are immunocompromised are those that have blood cell counts below the normal range. Normal ranges of blood counts are known to the medical practitioner and reference can be made to a standard hematology textbook for such counts. In addition, reference can be made to published PCT application PCT/US00/14505.

As mentioned above, the subject may have or be at risk of developing an infectious disease. The agents of the invention thus can be used to prevent or treat infectious diseases such as bacterial, viral, fungal, parasitic and myobacterial infections. The disclosed vaccine composition can also be used prophylactically to prevent or reduce the incidence of infection during periods of heightened risk, including for example flu season, epidemics, and travel to places where the risk of pathogen exposure is high. Vaccine compositions can also prepare a subject for passive exposure to a pathogen. Vaccine compositions also may be used to treat a subject who has an infection which is or has become resistant to one or more conventional drug therapies.

Antigens associated with infectious diseases that can be used in the methods of the invention include whole bacteria, whole virus, whole fungi, whole parasites, fragments thereof, lysates thereof, killed versions thereof, etc. TIM-3 activating agents can be used in combination with various vaccines either currently being used or in development, whether intended for human or non-human subjects. Examples of vaccines for human subjects and directed to infectious diseases include the combined diphtheria and tetanus toxoids vaccine; pertussis whole cell vaccine; the inactivated influenza vaccine; the 23-valent pneumococcal vaccine; the live measles vaccine; the live mumps vaccine; live rubella vaccine; Bacille Calmette-Guerin (BCG) tuberculosis vaccine; hepatitis A vaccine; hepatitis B vaccine; hepatitis C vaccine; rabies vaccine (e.g., human diploid cell vaccine); inactivated polio vaccine; meningococcal polysaccharide vaccine; quadrivalent meningococcal vaccine; yellow fever live virus vaccine; typhoid killed whole cell vaccine; cholera vaccine; Japanese B encephalitis killed virus vaccine; adenovirus vaccine; cytomegalovirus vaccine; rotavirus vaccine; varicella vaccine; anthrax vaccine; small pox vaccine.

Examples of bacterial infections include E. coli, Streptococcal infections, Staphylococcal infections, Pseudomonas infections, Clostridium difficile, Legionella infections, Pneumococcus infection, Haemophilus infections (e.g., Haemophilus influenzae infections), Klebsiella infections, Enterobacter infections, Citrobacter infections, Neisseria infections (e.g., N. meningitidis infection, N. gonorrhoeae infection), Shigella infections, Salmonella infections, Listeria infections (e.g., L. monocytogenes infection), Pasteurella infection (e.g., Pasteurella multocida infection), Streptobacillus infection, Spirillum infection, Treponema infection (e.g., Treponema pallidum infection), Actinomyces infection (e.g., Actinomyces israelli infection), Borrelia infection, Corynebacterium infection, Nocardia infection, Gardnerella infections (e.g., Gardnerella vaginalis infection), Campylobacter infections (e.g., Campylobacter fetus infection), Spirochaeta infections, Proteus infections, Bacteriodes infections, H. pylori, and anthrax.

Examples of viral infections include HIV infection, Herpes simplex virus 1 and 2 infections (including encephalitis, neonatal and genital forms), human papilloma virus infection, cytomegalovirus infection, Epstein Barr virus infection, Hepatitis virus A, B and C infections, rotavirus infection, adenovirus infection, influenza A virus infection, respiratory syncytial virus infection, varicella-zoster virus infections, small pox infection, monkey pox infection, and SARS infection. In some embodiments, the methods are not intended to treat or prevent HIV infection.

Examples of fungal infections include candidiasis infection, ringworm, histoplasmosis infection, blastomycosis infections, paracoccidioidomycosis infections, crytococcosis infections, aspergillosis infections, chromomycosis infections, mycetoma infections, pseudallescheriasis infection, and tinea versicolor infection.

Examples of parasite infections include both protozoan infections and nematode infections. These include amebiasis, Trypanosoma cruzi infection (i.e., Chagas' disease), Fascioliasis (e.g., Facioloa hepatica infection), Leishmaniasis, Plasmodium infections (e.g., malaria causing Plasmodium species infections, e.g., P. falciparum, P. knowlesi, P. malariae), Onchocerciasis, Paragonimiasis, Trypanosoma brucei infection (i.e., Sleeping sickness), Pneumocystis infection (e.g., Pneumocystis carinii infection), Trichomonas vaginalis infection, Taenia infections, Hymenolepsis infections (e.g., Hymenolepsis nana infection), Echinococcus infections, Schistosomiasis (e.g., Schistosoma mansoni infection), neurocysticercosis, Necator americanus infection, and Trichuris trichuria infections.

Other infections that can be treated according to the methods of the invention include Chlamydia infection, mycobacterial infection such as tuberculosis and leprosy, and Rickettsiae. The foregoing lists of infections are not intended to be exhaustive but rather exemplary. Those of ordinary skill in the art will identify other infections that are amenable to prevention and treatment using the methods of the invention.

Subjects having an infectious disease are those that exhibit symptoms of infectious disease (e.g., rapid onset, fever, chills, myalgia, photophobia, pharyngitis, acute lymphadenopathy, splenomegaly, gastrointestinal upset, leukocytosis or leukopenia) and in whom infectious pathogens or byproducts thereof can be detected. Tests for diagnosing infectious diseases are known in the art and the ordinary medical practitioner will be familiar with these laboratory tests which include but are not limited to microscopic analyses, cultivation dependent tests (such as cultures), and nucleic acid detection tests. These include wet mounts, stain-enhanced microscopy, immune microscopy (e.g., FISH), hybridization microscopy, particle agglutination, enzyme-linked immunoadsorbent assays, urine screening tests, DNA probe hybridization, serologic tests, etc. The medical practitioner will generally also take a full history and conduct a complete physical examination in addition to running the laboratory tests listed above.

A subject at risk of developing an infectious disease is one that is at risk of exposure to an infectious pathogen. Such subjects include those that live in an area where such pathogens are known to exist and where such infections are common. These subjects also include those that engage in high risk activities such as sharing of needles, engaging in unprotected sexual activity, routine contact with infected samples of subjects (e.g., medical practitioners), people who have undergone surgery, including but not limited to abdominal surgery, etc.

The agents of the invention are also indicated for treatment of human papillomavirus (HPV) infection. The current therapy for HPV is injection of IFN into a lesion and/or surgical ablation. A systemic treatment such as that envisioned for agents of the invention would be desirable in comparison with current clinical therapies. Agents of the invention are similarly useful in combination with HPV vaccines currently in development such as HPV virus-like particle (VLP)-based vaccine (see, for example, Virology 2000 Jan. 20; 266 (2):237-45).

In embodiments relating to the treatment of infectious disease, the treatments and vaccine compositions provided herein thus can further include anti-microbials agents. Examples of anti-microbials include anti-bacterials, anti-mycobacterials, anti-virals, anti-fungal, and anti-parasites.

Examples of anti-bacterials include β-lactam antibiotics, penicillins (such as natural penicillins, aminopenicillins, penicillinase-resistant penicillins, carboxy penicillins, ureido penicillins), cephalosporins (first generation, second generation, and third generation cephalosporins), and other β-lactams (such as imipenem, monobactams), β-lactamase inhibitors, vancomycin, aminoglycosides and spectinomycin, tetracyclines, chloramphenicol, erythromycin, lincomycin, clindamycin, rifampin, metronidazole, polymyxins, sulfonamides and trimethoprim, and quinolines.

Anti-mycobacterials include Myambutol (Ethambutol Hydrochloride), Dapsone (4,4′-diaminodiphenylsulfone), Paser Granules (aminosalicylic acid granules), Priftin (rifapentine), Pyrazinamide, Isoniazid, Rifadin (Rifampin), Rifadin IV, Rifamate (Rifampin and Isoniazid), Rifater (Rifampin, Isoniazid, and Pyrazinamide), Streptomycin Sulfate and Trecator-SC (Ethionamide).

Anti-virals include amantidine and rimantadine, ribivarin, acyclovir, vidarabine, trifluorothymidine, ganciclovir, zidovudine, retinovir, and interferons. Anti-virals further include: Acemannan; Acyclovir; Acyclovir Sodium; Adefovir; Alovudine; Alvircept Sudotox; Amantadine Hydrochloride; Aranotin; Arildone; Atevirdine Mesylate; Avridine; Cidofovir; Cipamfylline; Cytarabine Hydrochloride; Delavirdine Mesylate; Desciclovir; Didanosine; Disoxaril; Edoxudine; Enviradene; Enviroxime; Famciclovir; Famotine Hydrochloride; Fiacitabine; Fialuridine; Fosarilate; Foscarnet Sodium; Fosfonet Sodium; Ganciclovir; Ganciclovir Sodium; Idoxuridine; Kethoxal; Lamivudine; Lobucavir; Memotine Hydrochloride; Methisazone; Nevirapine; Penciclovir; Pirodavir; Ribavirin; Rimantadine Hydrochloride; Saquinavir Mesylate; Somantadine Hydrochloride; Sorivudine; Statolon; Stavudine; Tilorone Hydrochloride; Trifluridine; Valacyclovir Hydrochloride; Vidarabine; Vidarabine Phosphate; Vidarabine Sodium Phosphate; Viroxime; Zalcitabine; Zidovudine; Zinviroxime and integrase inhibitors.

Anti-fungals include imidazoles and triazoles, polyene macrolide antibiotics, griseofulvin, amphotericin B, and flucytosine. Antiparasites include heavy metals, antimalarial quinolines, folate antagonists, nitroimidazoles, benzimidazoles, avermectins, praxiquantel, ornithine decarboxylase inhibitors, phenols (e.g., bithionol, niclosamide); synthetic alkaloid (e.g., dehydroemetine); piperazines (e.g., diethylcarbamazine); acetanilide (e.g., diloxanide furonate); halogenated quinolines (e.g., iodoquinol (diiodohydroxyquin)); nitrofurans (e.g., nifurtimox); diamidines (e.g., pentamidine); tetrahydropyrimidine (e.g., pyrantel pamoate); sulfated naphthylamine (e.g., suramin).

Other anti-infectives include Difloxacin Hydrochloride; Lauryl Isoquinolinium Bromide; Moxalactam Disodium; Ornidazole; Pentisomicin; Sarafloxacin Hydrochloride; Protease inhibitors of HIV and other retroviruses; Integrase Inhibitors of HIV and other retroviruses; Cefaclor (Ceclor); Acyclovir (Zovirax); Norfloxacin (Noroxin); Cefoxitin (Mefoxin); Cefuroxime axetil (Ceftin); Ciprofloxacin (Cipro); Aminacrine Hydrochloride; Benzethonium Chloride: Bithionolate Sodium; Bromchlorenone; Carbamide Peroxide; Cetalkonium Chloride; Cetylpyridinium Chloride: Chlorhexidine Hydrochloride; Clioquinol; Domiphen Bromide; Fenticlor; Fludazonium Chloride; Fuchsin, Basic; Furazolidone; Gentian Violet; Halquinols; Hexachlorophene: Hydrogen Peroxide; Ichthammol; Imidecyl Iodine; Iodine; Isopropyl Alcohol; Mafenide Acetate; Meralein Sodium; Mercufenol Chloride; Mercury, Ammoniated; Methylbenzethonium Chloride; Nitrofurazone; Nitromersol; Octenidine Hydrochloride; Oxychlorosene; Oxychlorosene Sodium; Parachlorophenol, Camphorated; Potassium Permanganate; Povidone-Iodine; Sepazonium Chloride; Silver Nitrate; Sulfadiazine, Silver; Symclosene; Thimerfonate Sodium; Thimerosal: Troclosene Potassium.

The vaccine compositions also are used to treat subjects having or at risk of developing cancer. The invention is used to treat cancers that are immunogenic. Cancers that are immunogenic are cancers that are known to (or likely to) express immunogens on their surface or upon cell death. These immunogens are in vivo endogenous sources of cancer antigens and their release can be exploited by the methods of the invention in order to treat the cancer. In some embodiments, the vaccine composition comprises a cancer antigen as previously disclosed herein. In embodiments relating to the treatment of cancer, the methods and vaccine compositions provided herein can further include anti-cancer agents, including those previously disclosed herein.

The invention also seeks to enhance other forms of immunotherapy including dendritic cell vaccines. These vaccines generally include dendritic cells loaded ex vivo with antigens such as tumor-associated antigens. The dendritic cells can be incubated with the antigen, thereby allowing for antigen processing and expression on the cell surface, or the cells may simply be combined with the antigen prior to injection in vivo. Alternatively, the dendritic cells may be activated in vitro and then re-infused into a subject in the activated state.

The application further provides vaccine compositions that can be combined with dendritic cells in all of these embodiments. Examples of dendritic cell based vaccines include autologous tumour antigen-pulsed dendritic cells (advanced gynecological malignancies); blood-derived dendritic cells loaded ex vivo with prostate cancer antigen (Provenge; Dendreon Corporation); blood-derived dendritic cells loaded ex vivo with antigen for multiple myeloma and other B-cell malignancies (Mylovenge; Dendreon Corporation); and blood-derived dendritic cells loaded ex vivo with antigen for cancers expressing the HER-2/neu proto-oncogene (APC8024; Dendreon Corporation); xenoantigen (e.g., PAP) loaded dendritic cells, and the like.

The agent that increases TIM-3 activity may be used in combination therapies with one or more additional agents to enhance an immune response against tumor, viral or bacterial antigens. For example, CD40 binding proteins, which enhance the ability of dendritic cells to process and present antigens to effector T cells can be administered in combination with an agent that increases TIM-3 activity to dramatically enhance an immune response. Such immune responses can include responses against viral or bacterial antigens that are responsible for infectious diseases and immune responses to tumor antigens. Representative CD40 binding proteins useful in combination therapy with an agent that increases TIM-3 activity include CD40-L and antibodies immunoreactive with CD40 which are described in PCT publications WO 93/08207 and WO 96/40918.

Additionally, 4-1BB-L and antibodies reactive with 4-1BB, both of which are T-cell co-activation factors, can be administered in combination with the agent that increases TIM-3 activity to dramatically enhance immune responses. 4-1BB-L and antibodies reactive with 4-1BB can be used in combination therapies to enhance immune responses to viral antigens and bacterial antigens responsible for infectious diseases and to enhance immune responses to tumor antigens. 4-1BB-L and antibodies reactive with 4-1BB are described in U.S. Pat. No. 5,674,704.

Additionally, interferon alpha, RANKL, or a CD30 ligand antagonist can be administered in combination with agent that increases TIM-3 activity to dramatically enhance immune responses.

Immune responses can be induced or augmented by cytokines or chemokines (Bueler & Mulligan, 1996; Chow et al., 1997; Geissler et al., 1997; Iwasaki et al., 1997; Kim et al., 1997) or B-7 co-stimulatory molecules (Iwasaki et al., 1997; Tsuji et al., 1997). The cytokines and/or chemokines can be administered directly or may be administered in the form of a nucleic acid vector that encodes the cytokine, such that the cytokine can be expressed in vivo. In one embodiment, the cytokine or chemokine is administered in the form of a plasmid expression vector. The term “cytokine” is used as a generic name for a diverse group of soluble proteins and peptides which act as humoral regulators at nano- to picomolar concentrations and which, either under normal or pathological conditions, modulate the functional activities of individual cells and tissues. These proteins also mediate interactions between cells directly and regulate processes taking place in the extracellular environment. Cytokines also are central in directing the T cell response.

Examples of cytokines include, but are not limited to IL-1, IL-2, IL-4, IL-5, IL-6, IL-7, IL-10, IL-12, IL-15, IL-18, granulocyte-macrophage colony stimulating factor (GM-CSF), granulocyte colony stimulating factor (G-CSF), interferon-γ (IFN-γ), IFN-α, tumor necrosis factor (TNF), TGF-β, FLT-3 ligand, and CD40 ligand. In some embodiments, the cytokine is a Th1 cytokine. In still other embodiments, the cytokine is a Th2 cytokine.

The term “chemokine” is used as a generic name for peptides or polypeptides that act principally to chemoattract effector cells of both innate and adaptive immunity. Chemokines are thought to coordinate immunological defenses against tumors and infectious agents by concentrating neutrophils, macrophages, eosinophils and T and B lymphocytes at the anatomical site in which the tumor or infectious agent is present. In addition, many chemokines are known to activate the effector cells so that their immune functions (e.g., cytolysis of tumor cells) are enhanced on a per cell basis. Two groups of chemokines are distinguished according to the positions of the first two cysteine residues that are conserved in the amino-terminal portions of the polypeptides. The residues can either be adjacent or separated by one amino acid, thereby defining the CC and CXC cytokines respectively. The activity of each chemokine is restricted to particular effector cells, and this specificity results from a cognate interaction between the chemokine and a specific cell membrane receptor expressed by the effector cells. For example, the CXC chemokines IL-8, Groα/β and ENA 78 act specifically on neutrophils, whereas the CC chemokines RANTES, MIP-1α and MCP-3 act on monocytes and activated T cells. In addition, the CXC chemokine IP-10 appears to have anti-angiogenic activity against tumors as well as being a chemoattractant for activated T cells. MIP-1α also reportedly has effects on hemopoietic precursor.

Growth factors useful according to the invention include erythropoietin (U.S. Pat. No. 4,703,008) and analogs thereof, dipeptidylpeptidase inhibitors, Platelet Derived Growth Factor (PDGF) (U.S. Pat. No. 4,766,073), Platelet Derived Endothelial Cell Growth Factor (PD-ECGF) (U.S. Pat. No. 5,227,302), Human pituitary Growth Hormone (HGH) (U.S. Pat. No. 3,853,833), Transforming Growth Factor Beta (TGF.beta.) (U.S. Pat. No. 5,168,051), Transforming Growth Factor Alpha (TGF.alpha.) (U.S. Pat. No. 5,633,147), Keratinocyte Growth Factor (KGF) (U.S. Pat. No. 5,731,170), Insulin-like Growth Factor I (IGF-I) (U.S. Pat. No. 4,963,665), Epidermal Growth Factor (EGF) (U.S. Pat. No. 5,096,825), Erythropoietin (EPO), Granulocyte-Macrophage Colony Stimulating Factor (GM-CSF) (U.S. Pat. No. 5,200,327), M-CSF (U.S. Pat. No. 5,171,675), Colony Stimulating Factor-1 (CSF-1) (U.S. Pat. No. 4,847,201), Steel factor, Calcitonin, AP-1 proteins (U.S. Pat. No. 5,238,839), Brain Derived Neurotrophic Factor (BDNF) (U.S. Pat. No. 5,229,500). All of the references cited above are incorporated herein by reference in their entirety.

Other molecules that may be used in combination with agents that increase TIM-3 activity according to the present invention include IL-2, IL-12, IL-15, TRAIL, Fas ligand, VEGF antagonists, Tek antagonists, molecules that enhance dendritic cell function, survival, or expansion, molecules that enhance T cell activation or differentiation, molecules that enhance dendritic cell migration including various chemokines, molecules that increase the availability of target cell antigens, such as apoptotic factors and molecules that enhance MHC Class I presentation including the various interferons, angiogenesis inhibitors, inhibitors of immunosuppressive molecules released by tumors including IL-10, VEGF, and TGF-β, and tumor-specific antibodies including toxin- or radio-labeled antibodies.

In addition to stimulating an immune response to an antigen that already exists within the patient, the agent that increases TIM-3 activity may be administered prior to, concurrently with or subsequent to administration of an antigen to a patient for immunization purposes. Further, the agent that increases TIM-3 activity may be administered as a vaccine adjuvant in combination with additional active compounds prior to, concurrently with or subsequent to administration of an antigen to a patient for immunization purposes to enhance an immune response against tumor, viral or bacterial antigens. For example, CD40 binding proteins, such as CD40-L and antibodies to CD40 which enhance the ability of dendritic cells to present antigens to T cells can be administered in combination with agent that increases TIM-3 activity to dramatically enhance an immune response. Similarly, 4-1BB-L, antibodies reactive with 4-1BB, interferon alpha, RANKL, or CD30 ligand antagonists can be administered in combination with agent that increases TIM-3 activity to enhance an immune response and provide more effective immunization to the antigen.

For in vivo administration to humans, the agent that increases TIM-3 activity can be formulated according to known methods used to prepare pharmaceutically useful compositions. The agent that increases TIM-3 activity can be combined in admixture, either as the sole active material or with other known active materials (e.g. CD40 binding proteins, such as CD40-L or antibodies reactive with CD40, 4-1BB-L or antibodies reactive with 4-1BB, interferon alpha, RANKL, CD30 ligand antagonists), with pharmaceutically suitable diluents (e.g., Tris-HCl, acetate, phosphate), preservatives (e.g., Thimerosal, benzyl alcohol, parabens), emulsifiers, solubilizers, adjuvants and/or carriers. Suitable carriers and their formulations are described in Remington's Pharmaceutical Sciences, 16th ed. 1980, Mack Publishing Co. In addition, such compositions can contain the agent that increases TIM-3 activity complexed with polyethylene glycol (PEG), metal ions, or incorporated into polymeric compounds such as polyacetic acid, polyglycolic acid, hydrogels, etc., or incorporated into liposomes, microemulsions, micelles, unilamellar or multilamellar vesicles, erythrocyte ghosts or spheroblasts. Such compositions will influence the physical state, solubility, stability, rate of in vivo release, and rate of in vivo clearance of the agent that increases TIM-3 activity.

The agent that increases TIM-3 activity can be administered topically, parenterally, or by inhalation. The term “parenteral” includes subcutaneous injections, intravenous, intramuscular, intracisternal injection, or infusion techniques. These compositions will typically contain an effective amount of the agent that increases TIM-3 activity, alone or in combination with an effective amount of any other active material, e.g. those described above. Effective amounts, or dosages, and desired concentrations of the agent that increases TIM-3 activity and active compounds (e.g. CD40-L and/or 4-1BB-L, antibodies reactive with 4-1BB, interferon alpha, RANKL, CD30 ligand antagonists) contained in the compositions may vary depending upon many factors, including the intended use, patient's body weight and age, and route of administration. Preliminary doses can be determined according to animal tests, and the scaling of dosages for human administration can be performed according to art-accepted practices. Keeping the above description in mind, typical dosages of the agent that increases TIM-3 activity may range from about 10 μg per square meter to about 1000 μg per square meter. A preferred dose range is on the order of about 100 μg per square meter to about 300 μg per square meter.

### VIII. Formulations

The therapeutic agents described herein may be formulated into pharmaceutical compositions. Pharmaceutical compositions for use in accordance with the present invention may be formulated in conventional manner using one or more physiologically acceptable carriers or excipients. Thus, the compounds and their physiologically acceptable salts and solvates may be formulated for administration by, for example, by aerosol, intravenous, oral or topical route. The administration may comprise intralesional, intraperitoneal, subcutaneous, intramuscular or intravenous injection; infusion; liposome-mediated delivery; topical, intrathecal, gingival pocket, per rectum, intrabronchial, nasal, transmucosal, intestinal, oral, ocular or otic delivery.

An exemplary composition of the invention comprises an RNAi mixed with a delivery system, such as a liposome system, and optionally including an acceptable excipient. In a preferred embodiment, the composition is formulated for injection.

Techniques and formulations generally may be found in Remington's Pharmaceutical Sciences, Meade Publishing Co., Easton, Pa. For systemic administration, injection is preferred, including intramuscular, intravenous, intraperitoneal, and subcutaneous. For injection, the agents of the invention can be formulated in liquid solutions, preferably in physiologically compatible buffers such as Hank's solution or Ringer's solution. In addition, the agents may be formulated in solid form and redissolved or suspended immediately prior to use. Lyophilized forms are also included.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., ationd oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound. For buccal administration the compositions may take the form of tablets or lozenges formulated in conventional manner. For administration by inhalation, the agents for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration bile salts and fusidic acid derivatives. in addition, detergents may be used to facilitate permeation. Transmucosal administration may be through nasal sprays or using suppositories. For topical administration, the oligomers of the invention are formulated into ointments, salves, gels, or creams as generally known in the art. A wash solution can be used locally to treat an injury or inflammation to accelerate healing.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

For therapies involving the administration of nucleic acids, the oligomers of the invention can be formulated for a variety of modes of administration, including systemic and topical or localized administration. Techniques and formulations generally may be found in Remington's Pharmaceutical Sciences, Meade Publishing Co., Easton, Pa. For systemic administration, injection is preferred, including intramuscular, intravenous, intraperitoneal, intranodal, and subcutaneous for injection, the oligomers of the invention can be formulated in liquid solutions, preferably in physiologically compatible buffers such as Hank's solution or Ringer's solution. In addition, the oligomers may be formulated in solid form and redissolved or suspended immediately prior to use. Lyophilized forms are also included.

Systemic administration can also be by transmucosal or transdermal means, or the compounds can be administered orally. For transmucosal or transdermal administration, penetrants appropriate to the barrier to be permeated are used in the formulation. Such penetrants are generally known in the art, and include, for example, for transmucosal administration bile salts and fusidic acid derivatives. In addition, detergents may be used to facilitate permeation. Transmucosal administration may be through nasal sprays or using suppositories. For oral administration, the oligomers are formulated into conventional oral administration forms such as capsules, tablets, and tonics. For topical administration, the oligomers of the invention are formulated into ointments, salves, gels, or creams as generally known in the art.

Toxicity and therapeutic efficacy of the agents and compositions of the present invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

Dosage ranges for specific aspects of the invention are discussed separately above. To the extent that there is any question for a particular application, the dosage ranges recited for that application should prevail. However, for other applications or indications, dosages will generally range from about 0.001 to 100,000 μg/kg body weight of the subject.

In one embodiment of the methods described herein, the agent is administered at least once per day. In one embodiment, the agent is administered daily. In one embodiment, the agent is administered every other day. In one embodiment, the agent is administered every 6 to 8 days. In one embodiment, the agent is administered weekly.

As for the amount of the compound and/or agent for administration to the subject, one skilled in the art would know how to determine the appropriate amount. As used herein, a dose or amount would be one in sufficient quantities to either inhibit the disorder, treat the disorder, treat the subject or prevent the subject from becoming afflicted with the disorder. This amount may be considered an effective amount. In general, treatment is “effective” if one or more symptoms or markers of a disease or disorder is reduced by a statistically significant amount. Markers of a disease or disorder include, for example, markers which can be assayed for in a biological sample taken from the individual treated, and encompasses, e.g., a protein, a nucleic acid, a metabolite, an antigen, a cytokine, a carbohydrate, a lipid, or any other entity that can be measured as an indicator of a disease or disorder status. It is also contemplated that in some instances, an increase in a marker can be indicative of therapeutic efficacy. In such instances, a statistically significant increase in such marker is evidence of effective treatment. A person of ordinary skill in the art can perform simple titration experiments to determine what amount is required to treat the subject. The dose of the composition of the invention will vary depending on the subject and upon the particular route of administration used. Based upon the composition, the dose can be delivered continuously, such as by continuous pump, or at periodic intervals. For example, on one or more separate occasions. Desired time intervals of multiple doses of a particular composition can be determined without undue experimentation by one skilled in the art.

The effective amount may be based upon or affected by, among other things, the size of the compound, the biodegradability of the compound, the bioactivity of the compound and the bioavailability of the compound. If the compound does not degrade quickly, is bioavailable and highly active, a smaller amount will be required to be effective. The effective amount will be known to one of skill in the art; it will also be dependent upon the form of the compound, the size of the compound and the bioactivity of the compound. One of skill in the art could routinely perform empirical activity tests for a compound to determine the bioactivity in bioassays and thus determine the effective amount. In one embodiment of the above methods, the effective amount of the compound comprises from about 1.0 ng/kg to about 100 mg/kg body weight of the subject. In another embodiment of the above methods, the effective amount of the compound comprises from about 100 ng/kg to about 50 mg/kg body weight of the subject. In another embodiment of the above methods, the effective amount of the compound comprises from about 1 μg/kg to about 10 mg/kg body weight of the subject. In another embodiment of the above methods, the effective amount of the compound comprises from about 100 μg/kg to about 1 mg/kg body weight of the subject.

As for when the compound, compositions and/or agent is to be administered, one skilled in the art can determine when to administer such compound and/or agent. The administration may be constant for a certain period of time or periodic and at specific intervals. The compound may be delivered hourly, daily, weekly, monthly, yearly (e.g. in a time release form) or as a one time delivery. The delivery may be continuous delivery for a period of time, e.g. intravenous delivery. In one embodiment of the methods described herein, the agent is administered at least once per day. In one embodiment of the methods described herein, the agent is administered daily. In one embodiment of the methods described herein, the agent is administered every other day. In one embodiment of the methods described herein, the agent is administered every 6 to 8 days. In one embodiment of the methods described herein, the agent is administered weekly.

In some embodiments of the methods described herein in which an agent comprising a polypeptide is administered to a subject, the polypeptide is administered to the subject by administering a gene encoding such polypeptide. Expression constructs of the therapeutic polypeptides (such as a polypeptide comprising a wildtype or mutant TIM-3 IgV domain) may be administered in any biologically effective carrier, e.g. any formulation or composition capable of effectively transfecting cells in vivo with a recombinant fusion gene. Approaches include insertion of the subject fusion gene in viral vectors including recombinant retroviruses, adenovirus, adeno-associated virus, and herpes simplex virus-1, or recombinant bacterial or eukaryotic plasmids. Viral vectors can be used to transfect cells directly; plasmid DNA can be delivered with the help of, for example, cationic liposomes (lipofectin) or derivatized (e.g. antibody conjugated), polylysine conjugates, gramacidin S, artificial viral envelopes or other such intracellular carriers, as well as direct injection of the gene construct or CaPO₄ precipitation carried out in vivo. It will be appreciated that because transduction of appropriate target cells represents the critical first step in gene therapy, choice of the particular gene delivery system will depend on such factors as the phenotype of the intended target and the route of administration, e.g. locally or systemically. Additionally, molecules encoded within the viral vector, e.g., by a cDNA contained in the viral vector, are expressed efficiently in cells which have taken up viral vector nucleic acid.

Retrovirus vectors and adeno-associated virus vectors are generally understood to be the recombinant gene delivery system of choice for the transfer of exogenous genes in vivo, particularly into humans. These vectors provide efficient delivery of genes into cells, and the transferred nucleic acids are stably integrated into the chromosomal DNA of the host. A major prerequisite for the use of retroviruses is to ensure the safety of their use, particularly with regard to the possibility of the spread of wild-type virus in the cell population. The development of specialized cell lines (termed “packaging cells”) which produce only replication-defective retroviruses has increased the utility of retroviruses for gene therapy, and defective retroviruses are well characterized for use in gene transfer for gene therapy purposes (for a review see Miller, A. D. (1990) Blood 76:271). Thus, recombinant retrovirus can be constructed in which part of the retroviral coding sequence (gag, pol, env) has been replaced by nucleic acid encoding a CKI polypeptide, rendering the retrovirus replication defective. The replication defective retrovirus is then packaged into virions which can be used to infect a target cell through the use of a helper virus by standard techniques. Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14 and other standard laboratory manuals. Examples of suitable retroviruses include pLJ, pZIP, pWE and pEM which are well known to those skilled in the art. Examples of suitable packaging virus lines for preparing both ecotropic and amphotropic retroviral systems include ψCrip, ψCre, ψ2 and ψAm. Retroviruses have been used to introduce a variety of genes into many different cell types, including neural cells, epithelial cells, endothelial cells, lymphocytes, myoblasts, hepatocytes, bone marrow cells, in vitro and/or in vivo (see for example Eglitis, et al. (1985) Science 230:1395-1; Danos and Mulligan (1988) Proc. Natl. Acad. Sci. USA 85:6460-6464; Wilson et al. (1988) Proc. Natl. Acad. Sci. USA 85:3014-3018; Armentano et al. (1990) Proc. Natl. Acad. Sci. USA 87:6141-6145; Huber et al. (1991) Proc. Natl. Acad. Sci. USA 88:8039-8043; Ferry et al. (1991) Proc. Natl. Acad. Sci. USA 88:8377-8381; Chowdhury et al. (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol. 150:4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573).

Furthermore, it has been shown that it is possible to limit the infection spectrum of retroviruses and consequently of retroviral-based vectors, by modifying the viral packaging polypeptides on the surface of the viral particle (see, for example PCT publications WO93/25234, WO94/06920, and WO94/11524). For instance, strategies for the modification of the infection spectrum of retroviral vectors include: coupling antibodies specific for cell surface antigens to the viral env polypeptide (Roux et al. (1989) PNAS 86:9079-9083; Julan et al. (1992) J. Gen Virol 73:3251-3255; and Goud et al. (1983) Virology 163:251-254); or coupling cell surface ligands to the viral env polypeptides (Neda et al. (1991) J Biol Chem 266:14143-14146). Coupling can be in the form of the chemical cross-linking with a polypeptide or other variety (e.g. lactose to convert the env polypeptide to an asialoglycopolypeptide), as well as by generating fusion polypeptides (e.g. single-chain antibody/env fusion polypeptides). This technique, while useful to limit or otherwise direct the infection to certain tissue types, and can also be used to convert an ecotropic vector in to an amphotropic vector.

In addition to viral transfer methods, such as those illustrated above, non-viral methods can also be employed to cause expression of a the subject polypeptides in the tissue of an animal. Most nonviral methods of gene transfer rely on normal mechanisms used by mammalian cells for the uptake and intracellular transport of macromolecules. In preferred embodiments, non-viral gene delivery systems of the present invention rely on endocytic pathways for the uptake of the gene by the targeted cell. Exemplary gene delivery systems of this type include liposomal derived systems, poly-lysine conjugates, and artificial viral envelopes.

In a representative embodiment, a gene encoding one of the subject polypeptides can be entrapped in liposomes bearing positive charges on their surface (e.g., lipofectins) and (optionally) which are tagged with antibodies against cell surface antigens of the target tissue (Mizuno et al. (1992) No Shinkei Geka 20:547-551; PCT publication WO91/06309; Japanese patent application 1047381; and European patent publication EP-A-43075). For example, lipofection of neuroglioma cells can be carried out using liposomes tagged with monoclonal antibodies against glioma-associated antigen (Mizuno et al. (1992) Neurol. Med. Chir. 32:873-876).

In clinical settings, the gene delivery systems can be introduced into a patient by any of a number of methods, each of which is familiar in the art. For instance, a pharmaceutical preparation of the gene delivery system can be introduced systemically, e.g. by intravenous injection, and specific transduction of the target cells occurs predominantly from specificity of transfection provided by the gene delivery vehicle, cell-type or tissue-type expression due to the transcriptional regulatory sequences controlling expression of the gene, or a combination thereof. In other embodiments, initial delivery of the recombinant gene is more limited with introduction into the animal being quite localized. For example, the gene delivery vehicle can be introduced by catheter (see U.S. Pat. No. 5,328,470) or by stereotactic injection (e.g. Chen et al. (1994) PNAS 91: 3054-3057).

### Description of Nucleic Acid and Amino Acid Sequences Included Herein:

SEQ ID NO: 1 is TIM-3 Human Polypeptide. (Genbank Accession No. NP_{—}116171)
SEQ ID NO: 2 is a predicted TIM-3 homolog from Pongo pygmaeus (Genbank Accession No. CAH92001)
SEQ ID NO: 3 is a predicted TIM-3 homolog from Mus musculus (Genbank Accession No. NP_{—}599011)
SEQ ID NO: 4 is a predicted TIM-3 homolog from Bos taurus (Genbank Accession No. NP_{—}001070573)
SEQ ID NO: 5 is Galectin-9 Human Polypeptide. (Genbank Accession No. NP_{—}033665)
SEQ ID NO: 6 is an antisense oligonucleotide against TIM-3.

### The Sequences are as Follows:

SEQ ID NO: 1

MFSHLPFDCVLLLLLLLLTRSSEVEYRAEVGQNAYLPCFYTPAAPGNLVP
VCWGKGACPVFECGNVVLRTDERDVNYWTSRYWLNGDFRKGDVSLTIENV
TLADSGIYCCRIQIPGIMNDEKFNLKLVIKPAKVTPAPTLQRDFTAAFPR
MLTTRGHGPPAETQTLGSLPDINLTQISTLANELRDSRLANDLRDSGATI
RIGIYIGAGICAGLALALIFGALIFKWYSHSKEKIQNLSLISLANLPPSG
LANAVAEGIRSEENIYTIEENVYEVEEPNEYYCYVSSRQQPSQPLGCRFA
MP

SEQ ID NO: 2

MGEPQQVSALPPPPMQYIKEYTDENIQEGLAPKPPPPIKDSYMMFGNQFQ
CDDLIIRPLESQGIERLHPMQFDHKKELRKLNMSILINFLDLLDILIRSP
GNIKREEKLEDLKLLFHVVHHLINEYRPHQARETLRVMMEVQKRQRLETA
ERFQKHLERVIEVIQNCLASLPDDLPHSEAGMRVKTPEMDADDSNNCTGQ
NEHQRENSGSSEVKYIAEVGQNAYLPCFYTPAAPGNLVPVCWGKGACPVF
ECGSVVLRTDERDVNHRTSSRYWLNGDFRKGDVSLTIENVTLADSGIYCC
RIQIPGIMNDEKFNLKLVIKPAKVTPAPTLQRDFTAAFPRMLTTGGHGPA
ETQTPWSLRDINLTQIPTLDKELRDSGLANELRDSTIRIGIYIGAGISAG
LALALIFGALIFKWYSHSKEKIQNLSLISLANLPPSGLANAVAEGIRSEE
NIYTIEENVYEVEEPNEYYCYVSSGMKWRSPMSITAMSAVGSNPHNLWVV
ALQCHRSNHLIFELGVVFFRNYELCQLTGFGGSVHCYGAEFSHFQKIMTH
MGIELGPALNLNRHVIASVFKPTELLNPETVNHGCMAQSRLTVTSASWVQ
AILLPQPPEWLGLQACTTMPN

SEQ ID NO: 3

MFSGLTLNCVLLLLQLLLARSLEDGYKVEVGKNAYLPCSYTLPTSGTLVP
MCWGKGFCPWSQCTNELLRTDERNVTYQKSSRYQLKGDLNKGDVSLIIKN
VTLDDHGTYCCRIQFPGLMNDKKLELKLDIKAAKVTPAQTAHGDSTTASP
RTLTTERNGSETQTLVTLHNNNGTKISTWADEIKDSGETIRTAIHIGVGV
SAGLTLALIIGVLILKWYSCKKKKLSSLSLITLANLPPGGLANAGAVRIR
SEENIYTIEENVYEVENSNEYYCYVNSQQPS

SEQ ID NO: 4

MFSHLLFDCVLLMLLLLTSSLKGAYVSQVGQNADLPCTYSPATTENLVPV
CWGKGPCPVFECYSLVLRTDGRNVTYQTSSRYLLKRDLHKGDVTLTIKNV
TLADSGTYCCRIQFPGLMNDRKSNLELIIKPAKVTPAWTPWRDITTAFPR
MLTTKGPVSETRTLKTLHDKNQTEISTLATELQDMGATTRTGLYIGAGVF
AGLALILISGGLILKWYSDRKEKIQNSSLITLANLSPSGLANTAAEGMHP
VENIYIIEENIYEVEDPYECTCSVNSGHQS

SEQ ID NO: 5

MAFSGSQAPYLSPAVPFSGTIQGGLQDGLQITVNGTVLSSSGTRFAVNFQ
TGFSGNDIAFHFNPRFEDGGYVVCNTRQNGSWGPEERKTHMPFQKGMPFD
LCFLVQSSDFKVMVNGILFVQYFHRVPFHRVDTISVNGSVQLSYISFQNP
RTVPVQPAFSTVPFSQPVCFPPRPRGRRQKPPGVWPANPAPTIQTVIHTV
QSAPGQMFSTPAIPPMMYPHPAYPMPFITTILGGLYPSKSILLSGTVLPS
AQRFHINLCSGNHIAFHLNPRFDENAVVRNTQIDNSWGSEERSLPRKMPF
VRGQSGSVWILCEAHCLKVAVDGQHLFEYYHRLRNLPTINRLEVGGDIQL
THVQT

SEQ ID NO: 6

CAAACCAGGGUAUUCU

SEQ ID NO: 7

AGACACTGGTGACCCTCCATAATAACAATGGAA

SEQ ID NO: 8

CGGAGAGAAATGGTTCAGAGACA

SEQ ID NO: 9

TTCATCAGCCCATGTGGAAAT

The contents of any patents, patent applications, patent publications, or scientific articles referenced anywhere in this application are herein incorporated by reference in their entirety.

The invention now being generally described, it will be more readily understood by reference to the following examples, which are included merely for purposes of illustration of certain aspects and embodiments of the present invention, and are not intended to limit the invention, as one skilled in the art would recognize from the teachings hereinabove and the following examples, that, for example, other cell types, agents, constructs, or data analysis methods, all without limitation, can be employed without departing from the scope of the invention as claimed.

### EXAMPLES

### Methods

### Flow Cytometry

Single cell suspension from collagenase treated spleens were stained with the following antibodies: anti-CD11b, anti-CD11c, anti-TIM-3 (EBioscience) and Rat IgG1 isotype control (BD Biosciences). All data were collected on a FACSCalibur (BD Biosciences) and analyzed with FlowJo software (Tree Star).

### Real-Time Quantitative PCR

Total RNA was prepared from sorted cell populations using Trizol (Invitrogen) followed by RNA clean-up and DNaseI digestion (Qiagen). RNA was then reverse transcribed to cDNA and used as template in quantitative RT-PCR using the Taqman system (Applied Biosystems). Primers and probes for detection of full-length mouse TIM-3 were designed in the mucin domain. Sequences were as follows: Probe, 5′-AGA CAC TGG TGA CCC TCC ATA ATA ACA ATG GAA-3′ (SEQ ID NO:7); Forward primer, 5-CGG AGA GAA ATG GTT CAG AGA CA-3′ (SEQ ID NO:8); Reverse primer, 5-TTC ATC AGC CCA TGT GGA AAT-3′ (SEQ ID NO:9). GAPDH primers and probes were purchased from Applied Biosystems. Samples were run in duplicate. Data are expressed as expression relative to internal GAPDH control.

### In Vitro T Cell Differentiation

Naïve (CD4⁺CD62Lhigh) T cells were isolated from either wildtype or TIM3^{-/-} DO11.10 TCR transgenic mice by cell sorting on a BD FACSAria. Naïve T cells (1×10⁶) were cultured with 5×10⁶ irradiated splenic APC and OVA 323-339 peptide (50 μg/ml). On day 10, cells were harvested and cytokine production analyzed by intracytoplasmic cytokine staining according to manufacturer's protocol (BD Biosciences).

### Dendritic Cell Stimulation

Dendritic cells were isolated from collagenase digested spleens using CD11c magnetic beads (Miltenyi Biotech). DCs (3×10⁵/well) and D2SC1 cells (2.5×10⁵/well) were stimulated with 2 μg/ml recombinant human galectin-9 or 1 ng/ml LPS (Sigma). For stimulation with anti-TIM-3 or Mouse IgG1, tissue culture wells were pre-coated with 25 μg/ml goat anti-mouse IgG FC fragment specific antibody (Jackson Immunoresearch). 10 μg/ml of anti-TIM-3 or Mouse IgG1 were then added to coated wells and incubated. Unbound antibody was washed off prior to addition of D2SC1 cells. Supernatants were collected at 18 hours and cytokine production assessed by cytometric bead array (CBA) (BD Biosciences). For the NF-kB reporter assays, the D2SC1 dendritic cell line was plated at 3×10⁵ cells/well in 6-well plates and transfected with 1 μg of NF-κB-luciferase reporter plasmid construct using Fugene 6 (Roche Applied Science) according to manufacturer's instructions. Thirty six hours post transfection, cells were collected and re-seeded on plates coated with anti-mouse IgG (Fcγ-specific, 25 μg/ml, Jackson Immunolaboratories) and mouse IgG1 (eBioscience) or anti-TIM-3 monoclonal antibody 5D12 (10 μg/ml). After 6 and 24 hours, cells were lysed and luciferase activity was assayed using the Luciferase Assay System (Promega) according to manufacturer's instructions. Luciferase activity is displayed in relative units.

### Immunohistochemistry and Immunofluorescence

Frozen tissue sections were cut at 5 μm and fixed in acetone for 2 minutes. Antibodies to HLA-DR, CD80, CD86, PD-L1 (available from Dako) and against TIM-3 and galectin-9 (GalPharma, Japan) were incubated at optimal concentrations with white and grey matter tissue sections of normal brain of different ages. Secondary antibodies coupled to horseradish peroxidase (for IHC) or fluorochromes (for IF) were used to visualize the expression of these molecules on CD11b⁺ microglia.

### LCM and Quantitative RT-PCR.

To quantitate differences in gene expression, microglia from white and grey matter regions of normal brain or from inflamed CNS tissue samples were isolated by laser capture microdissection (LCM). Sections (5 μm) from frozen tissue samples were stained with anti-CD11b mAbs to identify microglia. Individual microglial cells were selected by laser capture (approximately 200 cells per sample) using a Pixcell system (Arcturus). RNA was extracted from LCM caps using the Absolutely RNA Nanoprep Kit (Stratagene). Complementary DNA was generated by reverse transcription, and following a pre-amplification step, TaqMan PCR was performed using primers/probes for TIM-3 and galectin-9. Each gene expression reaction was performed in duplicate; relative expression of these genes was calculated by comparison to the housekeeping genes β-2 microglobulin and GAPDH. Validated primers and probes for TaqMan RT-PCR were obtained from Applied Biosystems Gene Expression Assays except for analysis of human TIM-3 (Khademi et al., 2004), and the reaction conditions were optimized following the user's manual from the RNA UltraSense One-Step Quantitative RT-PCR kit (Invitrogen).

### FACS Isolation of Ex Vivo Human Microglia for Taqman Analysis of TIM-3

Single cell suspensions of MS plaques or tumor specimens were washed in PBS containing 1% human serum (FACS buffer) and stained for 30 minutes at 4° C. protected from light with optimal dilutions of dialzyed (azide-free) antibodies. Antibodies against CD11b and CD11c (BD Pharmingen) were used to identify and discriminate between microglia/macrophage and dendritic cell populations within the CNS, respectively. Cells were then washed and resuspended in 300 μl of buffer and sorted on a BD FACSAria (BD Biosciences).

### Function of Ex Vivo Human Monocytes

Human monocytes were isolated by negative selection from the peripheral blood of healthy subjects using magnetic beads (Miltenyi Biotech). Monocytes (2×10⁵/well) were stimulated with graded doses of LPS-free recombinant human galectin-9 in the presence of blocking TIM-3 antibody or isotype control to demonstrate specificity. Cytokine production was measured after 48 hours by ELISA.

### Induction of EAE

SJL mice were immunized with 100 μg of PLP 139-151 emulsified in either complete Freund's adjuvant (CFA) supplemented with Mycobacterium tuberculosis (4 mg/ml), incomplete Freund's adjuvant (IFA) containing either 100 μg of anti-TIM-3 (clone 5D12), Mouse IgG1 (Ebioscience) or IFA alone. Mice were also administered 100 ng pertussis toxin (List) intravenously on days 0 and 2. All antibodies used in vivo were LPS free. Mice were monitored daily for the development of disease which was scored according to the following scale: 0, no clinical signs; 1, loss of tail tone; 2, hindlimb weakness; 3, hindlimb paralysis; 4, forelimb paralysis; and 5, moribund or dead.

### Analysis of Murine Microglia

EAE was induced in SJL/J mice by immunization with 100 μg of PLP 139-151 emulsified in complete Freund's adjuvant (Difco). Each mouse also received 100 ng of Pertussis toxin intravenously on days 0 and 2. At different stages of disease, mice were sacrificed and CNS mononuclear cells obtained by percoll gradient centrifugation of collagenase digested CNS tissue (brain and spinal cord). Cells were then stained with antibodies to CD11b, CD45, TIM-3 or RatIgG1 isotype control and analyzed on a BD FACSCalibur.

### Experiment Series

### TIM-3 Expression on Dendritic Cells

### Example 1

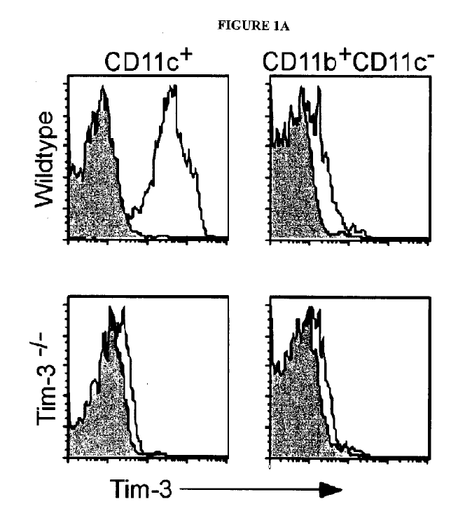

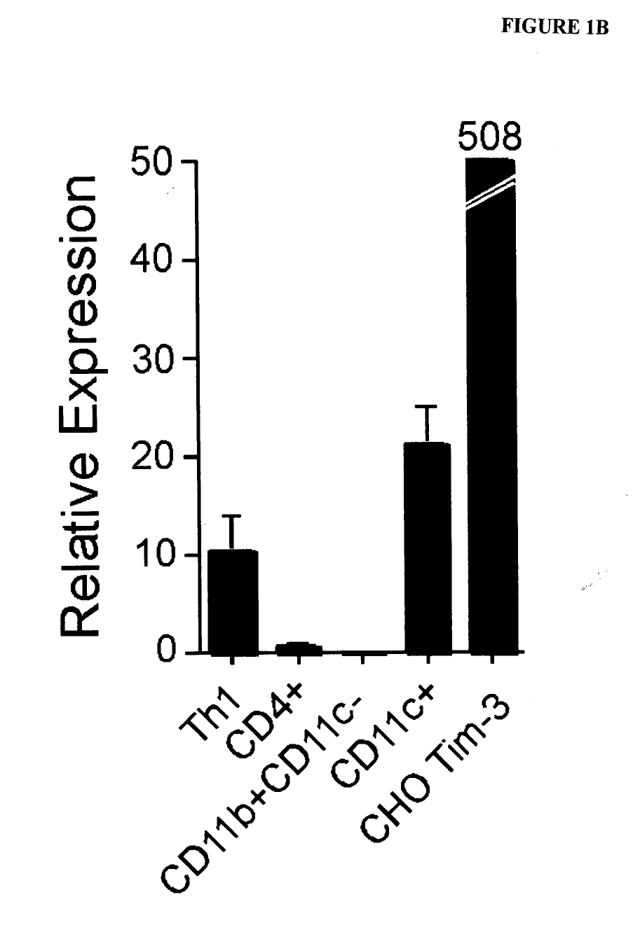

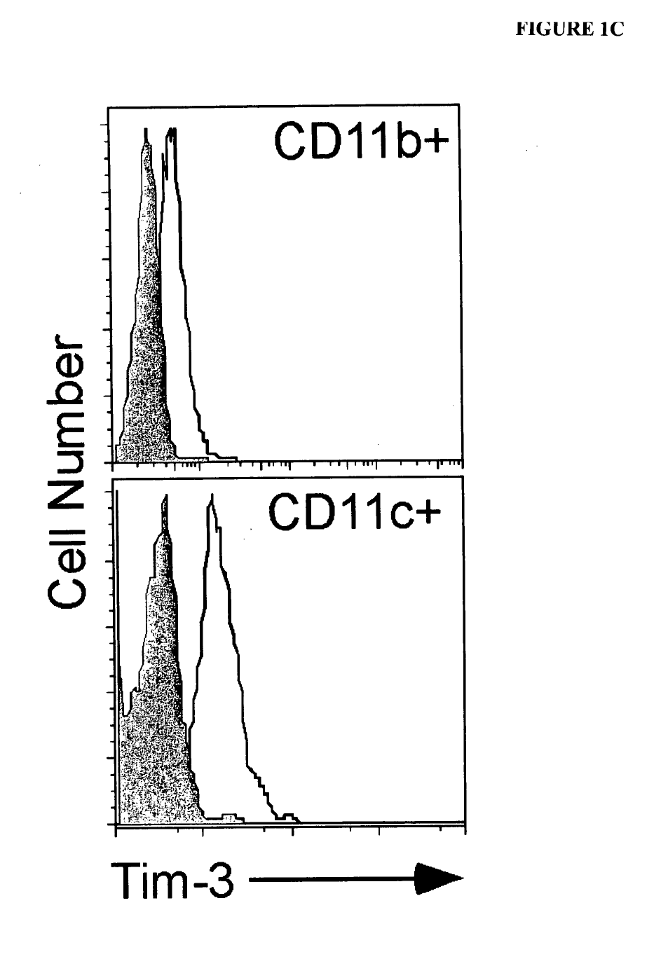

The expression of TIM-3 mRNA and protein in macrophages and myeloid dendritic cells (DCs) was examined. Murine macrophages (CD11b⁺CD11c⁻) did not express TIM-3, while nearly all CD11c⁺DCs expressed high levels of TIM-3 directly ex vivo (FIG. 1A). The staining of TIM-3 on DCs was specific, as no staining was observed on CD11c⁺ cells from TIM-3^{-/-} mice. The expression of TIM-3 on lymphoid (CD8⁺), myeloid (CD11b⁺), and plasmacytoid (B220⁺) DCs was also examined. TIM-3 was equally expressed on all subsets. TIM-3 was expressed at similar levels on immature versus mature bone marrow derived DCs.

### Example 2

Pure populations of both macrophages and dendritic cells were isolated by cell sorting, and examined for TIM-3 mRNA by quantitative RT-PCR. mRNA from ex vivo sorted CD4⁺ T cells, an activated Th1 T cell clone and CHO TIM-3 transfectants were used as controls. In agreement with the flow cytometry data, TIM-3 mRNA was present at high levels in dendritic cells and absent in macrophages (FIG. 1B).

### Example 3

The expression of human TIM-3 on ex vivo CD11b⁺ monocytes and CD11c⁺ myeloid DCs isolated from peripheral blood of healthy subjects was similarly evaluated (FIG. 1C). As in mice, human DCs expressed high levels of TIM-3. Interestingly, unlike murine macrophages, human monocytes constitutively expressed low levels of TIM-3. Thus, TIM-3 was highly expressed on DCs constitutively both in mice and man.

### Experiment Series

### TIM-3 on APCs Promotes Th1 Differentiation

### Example 4

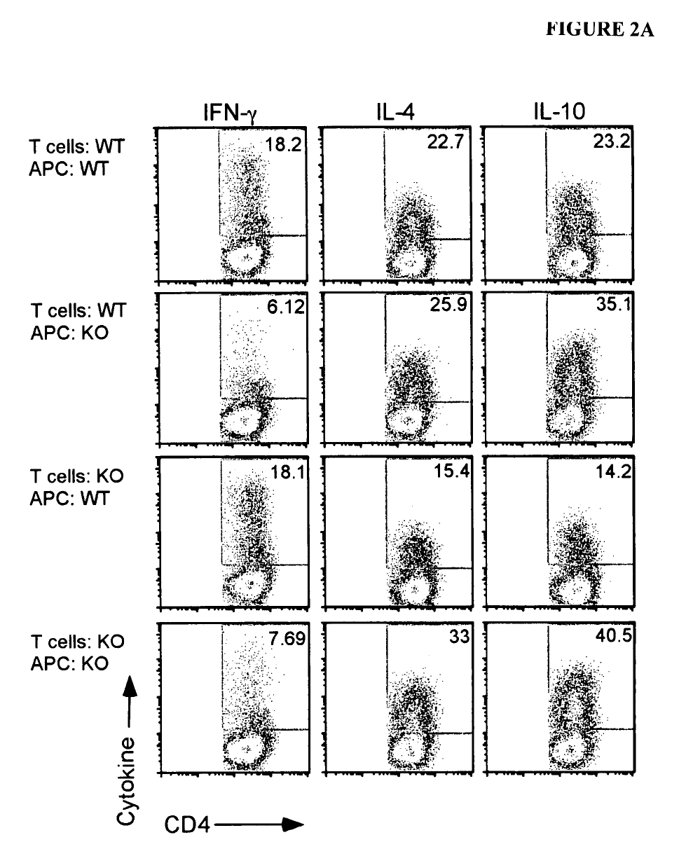

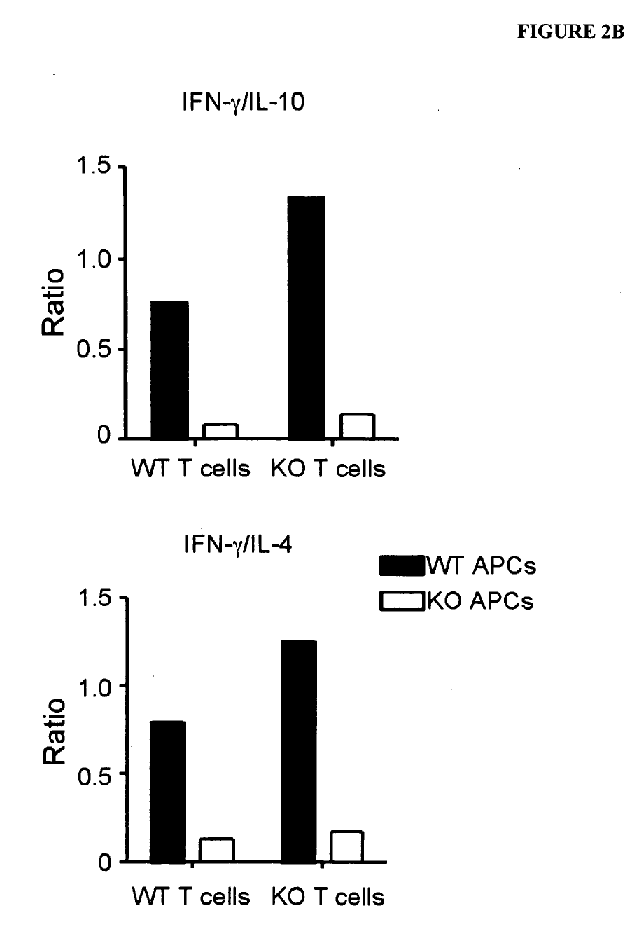

Naïve DO11.10 TCR transgenic T cells were differentiated under neutral conditions with peptide antigen and either wild type or TIM-3^{-/-} APCs. Surprisingly, DO11.10 TCR transgenic T cells produced less IFN-γ and more IL-4 and IL-10 after stimulation with TIM-3^{-/-} APCs (FIG. 2A). A similar Th phenotype was observed in parallel cultures with DO11.10 TCR transgenic TIM-3^{-/-} T cells (FIG. 2A). Differentiation towards a Th2 phenotype was driven by TIM-3 expression on APCs and not T cells, as it was only apparent when using TIM-3^{-/-} APCs and was equivalent when using wild type or TIM-3^{-/-} T cells (FIG. 2B).

### Experiment Series

### TIM-3 Function in Dendritic Cells

### Example 5

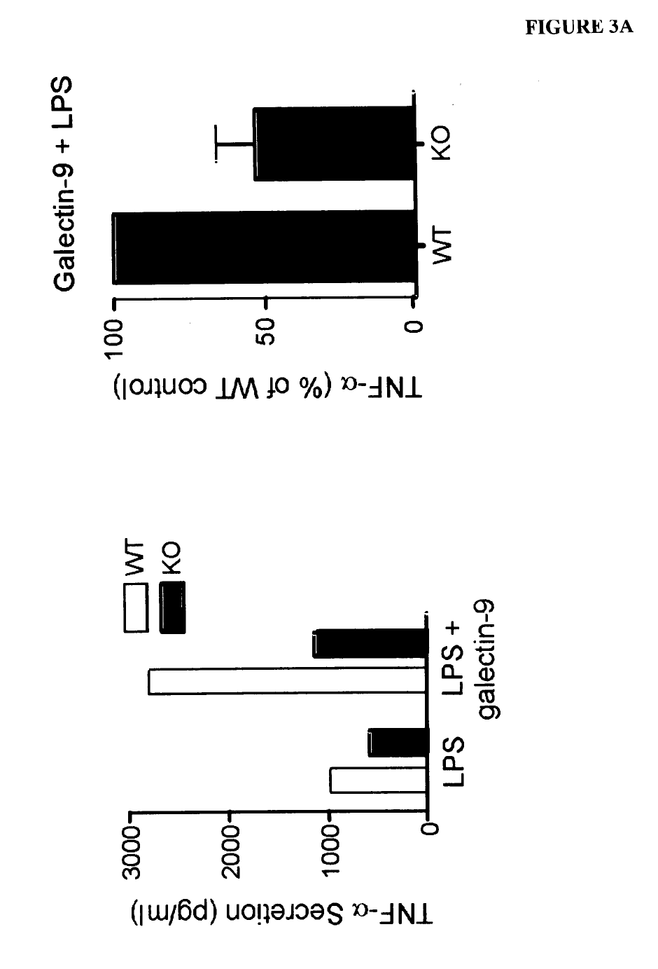

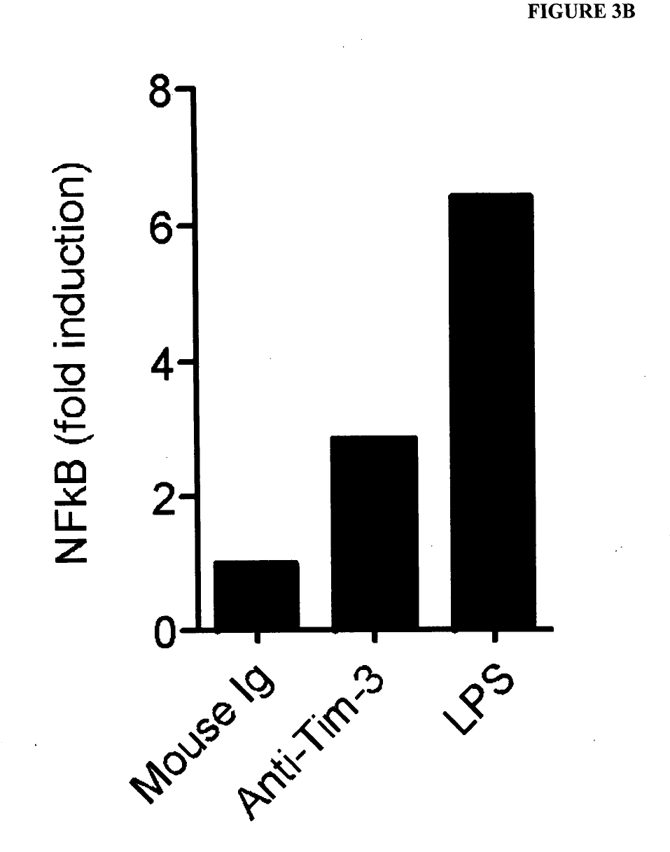

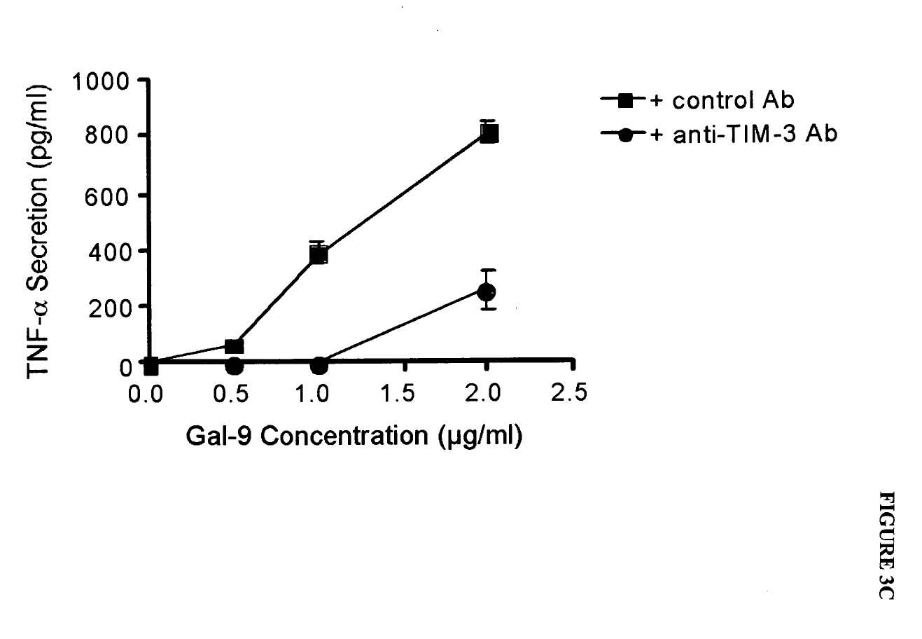

The production of cytokines from wild type and TIM-3^{-/-} DCs in response to galectin-9, LPS, or galectin-9 plus LPS was examined. While some TNF-α production was observed in response to galectin-9 stimulation alone, the production was low and not present in all experiments. However, galectin-9 consistently synergized with LPS to induce a much higher production of TNF-α in wild type DCs than LPS alone, and this synergistic effect was blunted in Tim3^{-/-} DCs (FIG. 3A). Tim3^{-/-} DCs consistently exhibited a blunted response to LPS as well (FIG. 3A). Indeed, the production of TNF-α in response to LPS and LPS plus galectin-9 was blunted by about 30-40% in TIM-3^{-/-} DCs relative to that in wild type DCs. IL-6 and IL-10 levels were also measured. No specific production of IL-10 and little difference in IL-6 production between wild type and TIM-3^{-/-} DCs was observed (data not shown).

### Example 6

An agonist anti-TIM-3 antibody was generated by immunizing TIM-3^{-/-} mice and screening for antibodies that specifically bound TIM-3 and inhibited both the proliferation and IFN-γ secretion of TIM-3⁺Th1 T cells. One anti-TIM-3 antibody (clone 5D12) was identified as exhibiting agonistic activity in T cells (data not shown).

### Example 7

To examine some of the molecular events associated with TIM-3 signaling in DCs, a dendritic cell line that expresses TIM-3 was used. The D2SC1 cell line naturally expresses TIM-3 (data not shown). DCs isolated ex vivo produced TNF-α in response to TIM-3 ligation. The induction of NF-κB was examined. To do this, D2SC1 cells were transfected with an NFκB reporter construct. The transfected cells were stimulated with agonistic anti-TIM-3 antibody and then assayed for luciferase activity. Consistent with the stimulation of this cell line with the agonistic anti-TIM-3 antibody also induced TNF2 (FIG. 3B) cytokine data, engagement of TIM-3 specifically induces NFκB (FIG. 3B).

### Example 8

Whether this effect was mediated by TIM-3 present on human monocytes was examined. Indeed, anti-TIM-3 antibody could inhibit galectin-9-mediated TNF-α secretion from human monocytes by 75% (FIG. 3C), further supporting the observation made in mice, where triggering TIM-3 by galectin-9 induced TNF-α secretion from DCs.

### Experiment Series

### TIM-3 Expression in Human Microglia in CNS

### Example 9

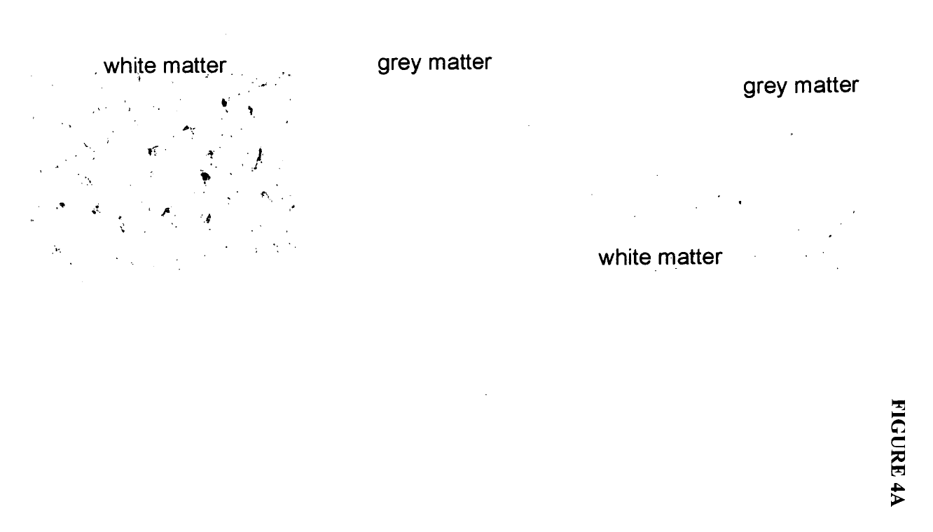

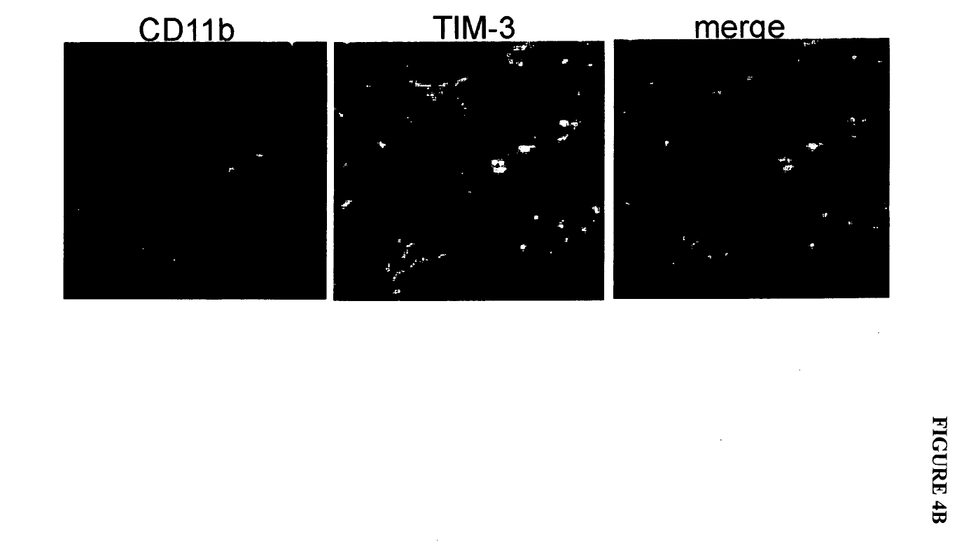

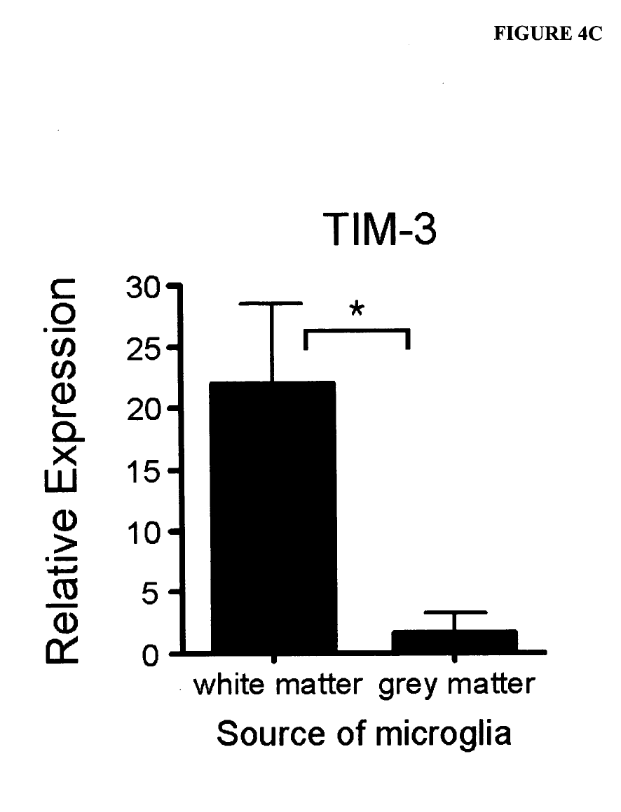

TIM-3 expression was examined in the CNS of fifteen brains obtained from autopsy of subjects with no known inflammatory disease. Surprisingly, robust TIM-3 staining was observed in white matter but not grey matter parenchyma on what appeared histologically to be microglia (FIG. 4A). This marked difference in TIM-3 expression in white versus grey matter regions was observed in the CNS tissues of all fifteen subjects examined. Dual immunofluorescence staining with antibodies against TIM-3 and CD11b confirmed the expression of TIM-3 on microglia in CNS white matter (FIG. 4B). Immunohistochemical staining of non-inflamed murine CNS tissue confirmed a selective expression of TIM-3 in white matter but not grey matter tissue (data not shown).

### Example 10

Some myeloid cell markers stain differently in white matter versus grey matter tissue. Accordingly, laser capture microdisection (LCM) and quantitative RT-PCR analysis of CD11b⁺ microglia from normal white and grey matter tissues was used to confirm the specific and selective expression of TIM-3 on white matter microglia. Consistent with the immunohistochemical staining with TIM-3 antibody, little or no TIM-3 mRNA was detected in microglia obtained from grey matter tissue whereas TIM-3 mRNA was observed in microglia obtained from white matter tissue (FIG. 4C).

### Example 11

To determine whether other cell surface determinants were differentially expressed on CNS microglia, immunohistochemical staining was used to analyze the expression of HLA class II and the costimulatory molecules CD80, CD86, and PDL1. Immunohistochemical staining for HLA-DR revealed comparable expression on both white matter and grey matter microglia (FIG. 4D), while there was little or no expression of CD80, CD86, and PDL1 on either white or grey matter microglia (data not shown).

### Experiment Series

### TIM-3 Expression in Microglia Differs Depending on the Nature of CNS Inflammation

### Example 12

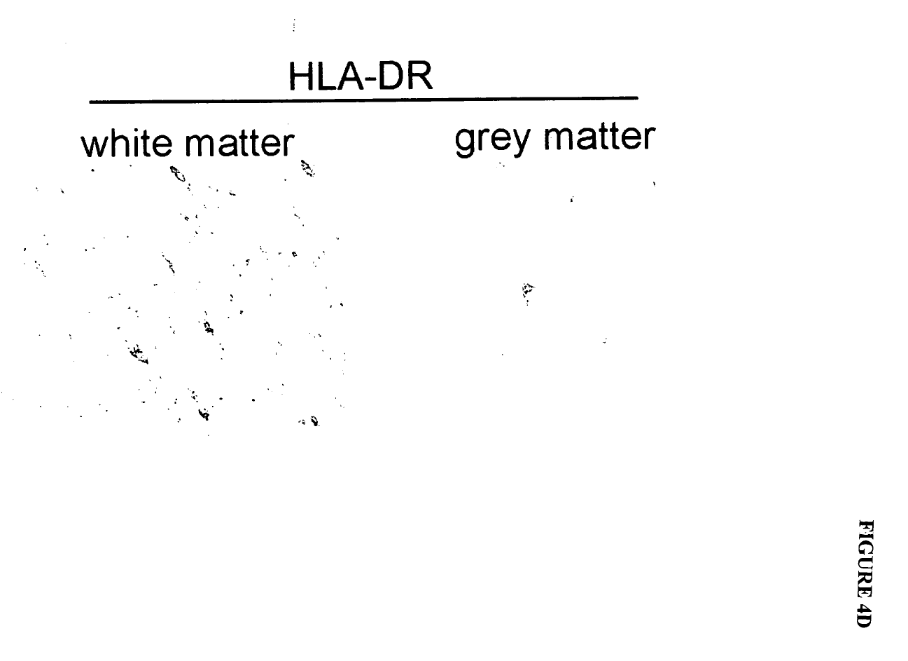

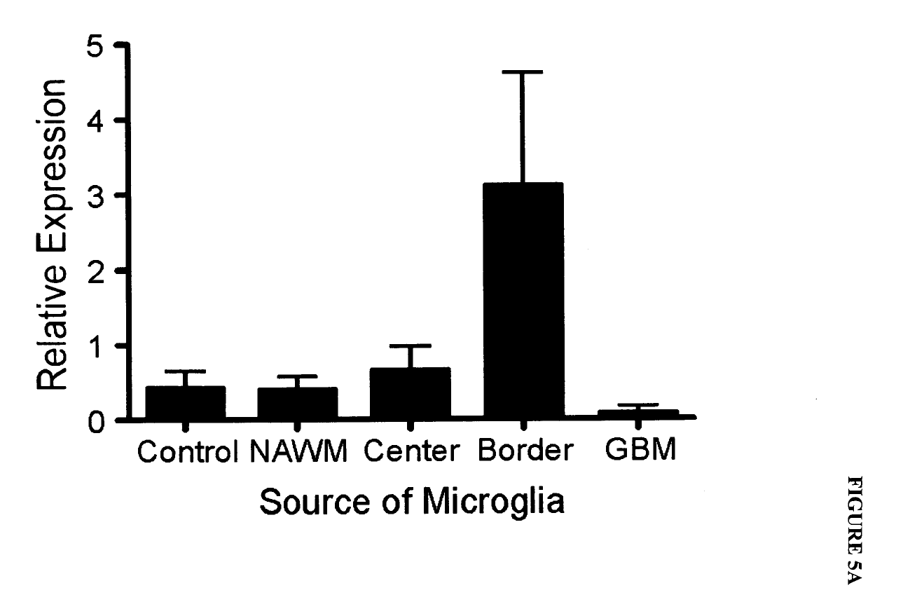

It was examined whether TIM-3 expression would be increased on microglia isolated from the CNS of autoimmune white matter tissue associated with heightened immune responses while decreased on microglia isolated from CNS tumors associated with a compromised response. Thus, the expression of TIM-3 was compared on microglia captured from MS and glioblastoma multiforme (GBM) brain tumor tissue specimens. Lymphocytes and microglia infiltrate both types of inflamed tissues, though the cytokine profiles differ considerably, with Th1 cytokines IFN-γ and TNF-α associated with MS but not GBM tissue infiltrates. Microglia captured from the active border regions of MS lesions expressed higher levels of TIM-3 than did those captured from the quiescent center of MS lesions, adjacent normal appearing white matter, or those obtained from non-inflamed control white matter tissue (FIG. 5A).

### Example 13

TIM-3 expression was significantly lower in microglia obtained from glioblastoma multiforme (GBM) tissues relative to those obtained from control tissue or MS tissue. Differences in TIM-3 expression in microglia obtained from MS and GBM tissue samples were confirmed by quantitative RT-PCR analysis of microglia isolated by fluorescence activated cell sorting (FACS) from viable tissue preparations (FIG. 5B). These data demonstrate an association between TIM-3 expression on microglia and Th1-type CNS inflammation.

### Example 14

Whether galectin-9 was up-regulated on astrocytes present in MS lesions was examined. Indeed, galectin-9 levels were significantly elevated on astrocytes present in MS lesions relative to normal human CNS tissue (FIG. 5C). Collectively, these data demonstrate that both TIM-3 and it's ligand galectin-9 can be up-regulated on glial cells (microglia and astrocytes, respectively) and induce TNF-α secretion. Moreover, they suggest that TIM-3 expression on white matter microglia may be an important means by which innate immunity within the CNS senses tissue inflammation.

Analysis of human and murine glial tumors has consistently demonstrated large numbers of tumor-infiltrating microglia that are widely distributed throughout and around the tumor (Badie et al., 2002, J. Neuroimmunol. 133: 39-45; Badie et al., 2000, Neurosurgery 46: 957-961; Deininger et al., 2001, J. Neuro-Oncology 55: 141-147; Morantz et al., 1979, J. Neurosurg. 50: 298-304; Roggendorf et al., 1996, Acta Neuropathol. 92: 288-293; Tran et al., 1998 J. Immunol. 161: 3767-3775), and indeed, in many cases microglia comprise over a third of the tumor mass (Morimura et al., 1990; Badie et al., 2000, supra; Morantz, 1979, supra. Emerging data suggest that microglia present in the tumor microenvironment are functionally impaired (Flugel et al., 1999, Int. J. Dev. Neurosci. 17: 547-556; Schartner et al., 2005 Glia 51: 279-285; Tran et al., 1998, supra]. While not wishing to be bound by theory, the present experiments indicate that suppression of the TIM-3/galectin-9 pathway may be a mechanism that inhibits the stimulatory and tumoricidal capacity of microglia that infiltrate glioblastomas. Given the central role of inflammation in other CNS diseases, it is contemplated that the TIM-3/galectin-9 pathway is important in other diseases, including, among others, those that more closely parallel the inflammation of MS, such as HIV-associated dementia or viral encepthalitis.

### Experiment Series

### Analysis of TIM-3 on Murine Monocytes/Macrophages and Microglia

### Example 15

There are a number of potential explanations for the selective expression of TIM-3 on white matter microglia but not gray matter microglia in the non-inflamed CNS, including selective migration of TIM-3⁺ monocytes from the blood into white matter regions of the CNS or factors present in white matter or gray matter regions that induce or suppress TIM-3 expression, respectively, on microglia. To investigate these possibilities, Applicants examined the expression of Tim-3 on CD11b⁺ macrophages isolated from the spleens of naïve wild type and Tim-3-deficient mice. In contrast to microglia, peripheral macrophages were uniformly negative for Tim-3.

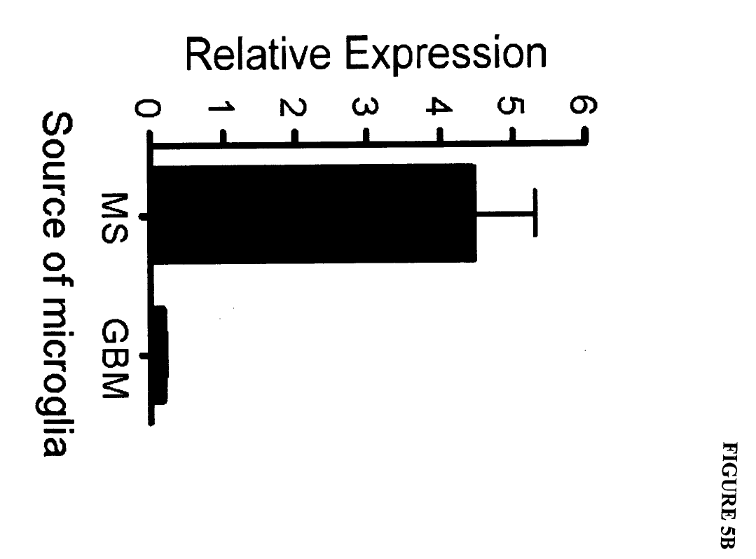

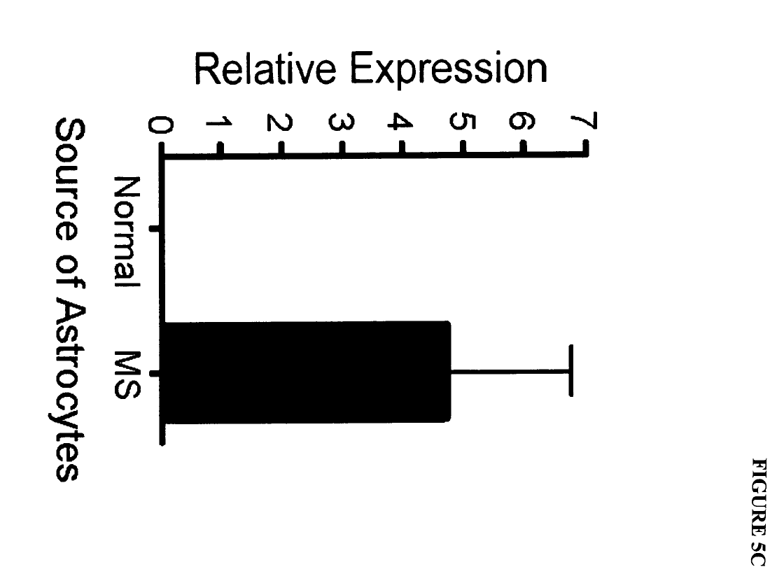

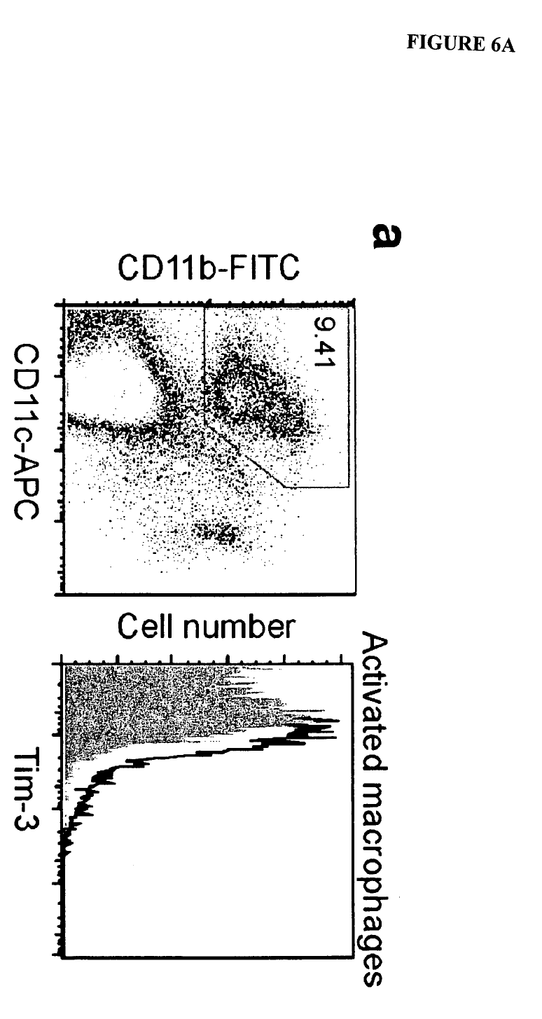

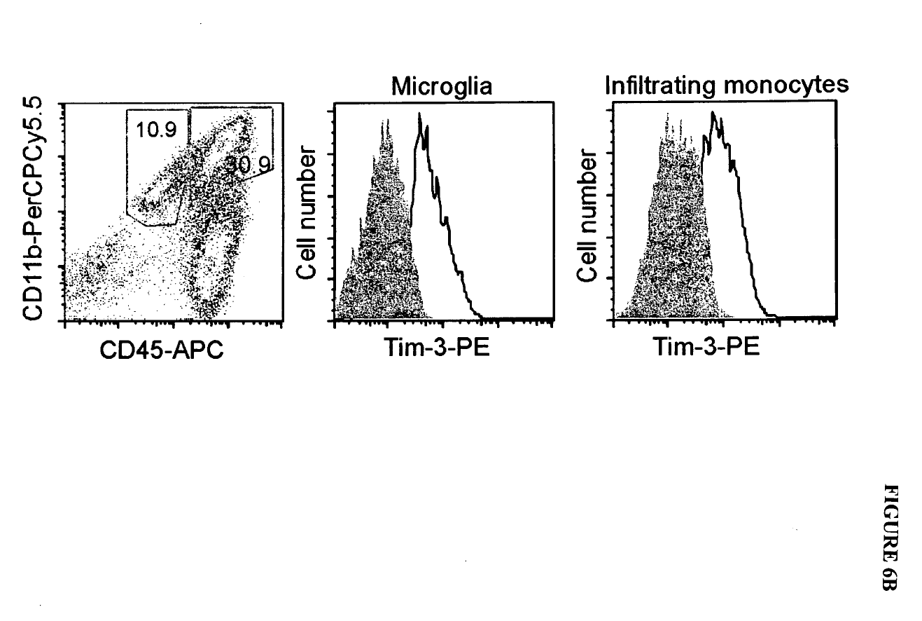

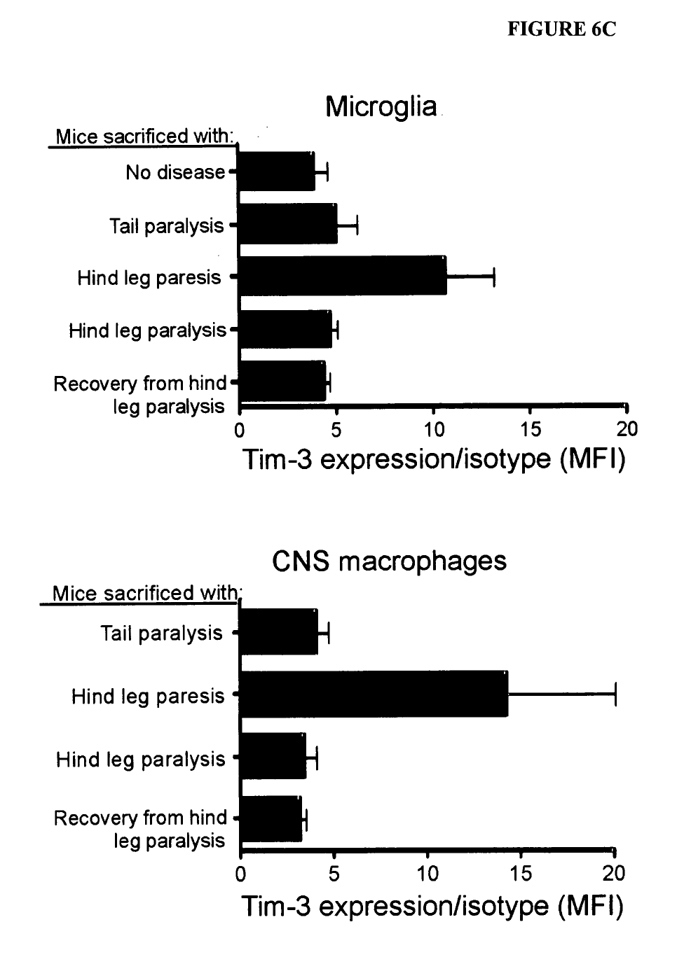

Mice were immunized with myelin proteolipid protein, PLP 139-151, to induce experimental autoimmune encephalomyelitis (EAE), a murine model of MS, and analyzed TIM-3 expression on peripheral macrophages on day 5 after disease induction. Activated CD11b⁺ cells failed to up-regulate TIM-3 expression (FIG. 6A). Surprisingly however, CD11b⁺ monocytes that infiltrated the CNS from the periphery, distinguished from resident microglia by higher expression of CD45 (Ford, A. L. et. al., J Immunol 154, 4309-21 (1995) and Williams, K. et al., J Neuropathol Exp Neurol 51, 538-49 (1992)), did express TIM-3 (FIG. 6B). Moreover, levels of TIM-3 on both microglia and infiltrating monocytes were comparable, increasing with severity of disease, and peaking just prior to the peak of clinical disease (FIG. 6C).

As peripheral macrophages are uniformly negative for Tim-3 but uniformly positive for Tim-3 upon entry into the CNS, these data strongly suggest that factor(s) present in the CNS can induce Tim-3 expression on resident and infiltrating monocytes. Indeed, heterogeneity in the morphology of microglia throughout the CNS has been attributed to sensitivity of these cells to regional differences in their microenvironments (Lawson et al., 1990), and these data demonstrate that expression of TIM-3 is similarly regulated by regional differences.

While there is a small degree of gray matter involvement, MS is predominantly a disease of inflammation in the CNS white matter. It has been widely assumed that MS is a white matter disease because antigen specific cells recognize their antigens predominantly within CNS white matter. While Applicants believe this is fundamentally true, the data described herein raise an additional option. While not wishing to be bound by theory, myelin-reactive Th1 cells may enter white matter regions of the CNS and recognize myelin antigens, where they secrete IFN-γ, inducing high levels of galectin-9 on astrocytes. Expression of galectin-9 on reactive astrocytes may then engage TIM-3 on the surface of microglia and/or infiltrating monocytes in the white matter and induce TNF-α secretion, serving to promote inflammation and demyelination. This is in accord with a major therapeutic observation in the field; early relapsing/remitting patients respond to immunosuppression, while patients with secondary progressive disease, who may be in a degenerative phase not mediated by T cells, are refractory to immunotherapy. This phase of disease may be mediated by chronic activation of infiltrating monocytes/microglia by the TIM-3:galectin-9 axis. Indeed, recent evidence indeed suggest that an early event in secondary progressive MS is the activation of CNS microglia (Trapp et al., 2004). Furthermore, recent EAE studies have demonstrated the importance of microglia in the onset of disease (Heppner et al., 2005; Ponomarev et al., 2005).

In this vein, it is noted that the relapsing/remitting phase of multiple sclerosis is primarily a T-cell-driven process. That is, in relapsing/remitting MS, inappropriate T-cell activation, with its accompanying inflammation, is primarily responsible for the tissue damage. In the later, secondary, progressive stage of the disease, however, the pathology is not due primarily to T-cells, but rather, to inappropriate activities of antigen presenting cells. Without wishing to be bound by theory, it is noted that TIM-3 serves as a negative regulator of T cell activation, but as positive regulator of APC activation. Thus, in relapsing/remitting MS, one would not expect that inhibition of TIM-3 activity would be helpful therapeutically; rather, one might expect that inhibiting the natural inhibitory activity of TIM-3 would make the disease worse by removing a factor protecting from excessive inflammation. However, in secondary, progressive MS, where APC activation plays the predominant role, and where TIM-3 positively regulates APC activation, inhibition of TIM-3 activity would be effective to inhibit the progression of the disease. Therefore, the discovery that TIM-3 is expressed on, and positively regulates APC activation indicates that treatments based on TIM-3 modulation should only be administered to those in the secondary, progressive stage of MS. Treatment involving administration of TIM-3 inhibitors should therefore include, before such administration, a determination that the individual being treated is in the secondary, progressive stage of the disease. An ordinarily skilled clinician can determine whether MS is in the relapsing/remitting stage or in the secondary, progressive stage. Aside from the patient's history of symptoms, remissions and exacerbations, clinical markers and parameters indicative of disease stage are known to those skilled in the art. One example of how the disease stage can be determined from clinical markers is described by Jongen et al., 1997, J. Neurol. Neurosurg. Psychiatry 63: 446-451, which describes the evaluation of cerebrospinal fluid for a panel of markers, including, for example, albumin CSF: peripheral blood ratio, mononuclear cell number, CD4+, CD8+ and B1+ subsets, CD4+:CD8+ ratio, IgG, IgG index, IgM, IgM index, complement components C3 and C4, and C3 and C4 indices, myelin basic protein, neuron-specific enolase, S100 and lactate. The measurement of these parameters and markers permits reliable MS staging as described by the authors of the study, which is incorporated herein by reference.

### Experiment Series

### In Vivo Affect of Agonistic Anti-TIM-3 Antibody

### Example 16

Mice were immunized for the development of experimental autoimmune encephalomyelitis (EAE), an animal model of central nervous system (CNS) autoimmunity in which both Th1 cells and TNF-α play crucial roles in disease pathogenesis. Mice were immunized with PLP 139-151 emulsified in incomplete Freund's adjuvant (IFA) containing either agonistic anti-TIM-3 antibody or isotype control. Mice immunized with IFA containing agonistic anti-TIM-3 developed more severe disease than did mice immunized with IFA and control antibody (FIG. 7A). This observation supports the hypothesis that in the innate immune system, TIM-3 augments generation of Th1 immunity, but later can influence adaptive immunity when expressed on differentiated Th1 cells. Regression curve analysis indicated a highly significant difference in the disease course induced in the presence of agonistic anti-TIM-3 monoclonal antibody versus control antibody (FIG. 7B).

### Example 17

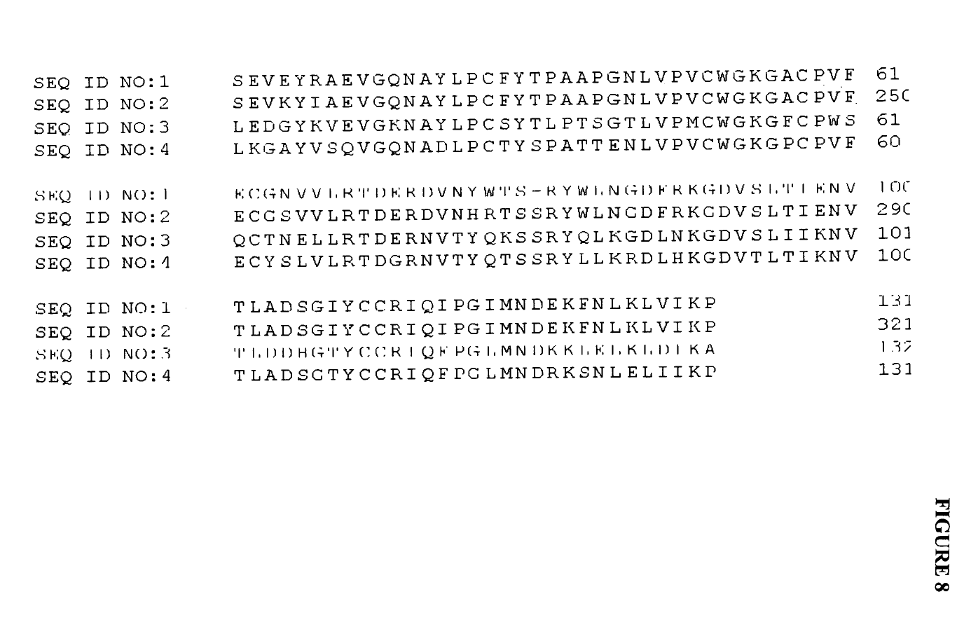

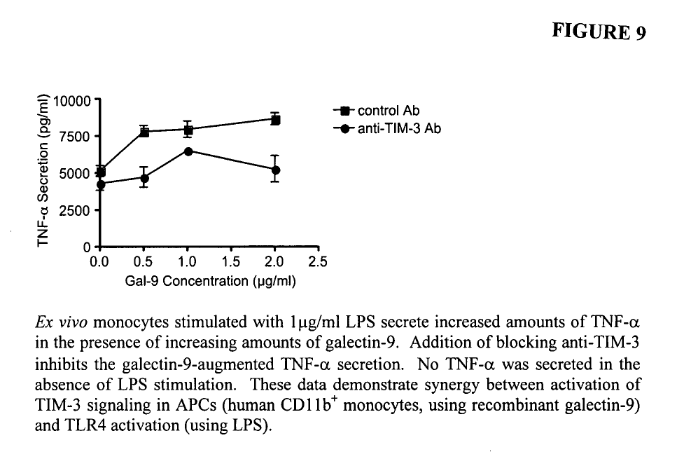

Ex vivo monocytes were stimulated with 1 μg/ml LPS in the absence and presence of increasing amounts galectin-9. It was found that the monocytes secreted increased amounts of TNF-α in the presence of increasing amounts of galectin-9 (FIG. 9). The addition of blocking anti-TIM-3 antibody inhibited the galectin-9-augmented TNF-α secretion (FIG. 9). No TNF-α was secreted in the absence of LPS stimulation. These data demonstrate synergy between activation of TIM-3 signaling in APCs (human CD11b⁺ monocytes, using recombinant galectin-9) and TLR4 activation (using LPS). Thus, TLR agonist can enhance immune responses involving TIM-3 activation.

### Other Embodiments

All patents, patent applications, and published references cited herein are hereby incorporated by reference in their entirety. While this invention has been particularly shown and described with references to preferred embodiments thereof, it will be understood by those skilled in the art that various changes in form and details may be made therein without departing from the scope of the invention encompassed by the appended claims.

1. A method of treating multiple sclerosis in a subject in need of such treatment, the method comprising a) assessing whether the subject is in the remitting/relapsing or the secondary, progressive stage of multiple sclerosis, and, b) if said subject is determined to be in the secondary, progressive stage of multiple sclerosis, administering to the subject a therapeutically effective amount of an agent that decreases TIM-3 activity in the subject.

2. The method of claim 1, wherein administration of the agent decreases the TIM-3 activity in antigen presenting cells (APCs).

3. The method of claim 2, wherein the APCs comprise i) CD1 Ib⁺ microglia cells, ii) CD1 1b+ monocytes, iii) dendritic cells (DCs), iii) or each of these populations.

4. The method of claim 3, wherein the CD1 1b⁺ microglia cells are located in the central nervous system.

5. (canceled)

6. The method of claim 1, wherein the subject is a human.

7. The method of claim 1, wherein the agent is an antibody or an antigen-binding fragment thereof.

8. The method of claim 7, wherein the antibody or fragment thereof binds to TIM-3.

9. (canceled)

10. The method of claim 9, wherein the agent is an antibody or antibody fragment that binds to a polypeptide comprising amino acids 30-128 of SEQ ID NO: 1.

11. The method of claim 1, wherein the agent reduces the binding of galectin-9 to TIM-3.

12. The method of claim 1, wherein the agent comprises a polypeptide comprising
(i) amino acids 30-128 of SEQ ID NO: 1; or
(ii) an amino acid sequence that is at least 90% identical to amino acids 30-128 of SEQ ID NO: 1 and which binds to galectin-9, inhibits release of TNF-α in APCs or both.

13. (canceled)

14. (canceled)

15. The method of claim 1, wherein the agent decreases the expression level a TIM-3 polypeptide or a galectin-9 polypeptide in the subject.

16. The method of claim 15, wherein the agent is a double stranded RNA oligonucleotide.

17. The method of claim 1, wherein the agent inhibits binding of full-length TIM-3 to a galectin-9 polypeptide.

18. The method of claim 1, wherein the agent inhibits binding of a polypeptide comprising amino acids 30-128 of SEQ ID NO: 1 to galectin-9.

19. The method of claim 18, wherein said galectin-9 polypeptide comprises the amino acid sequence set forth in SEQ ID NO:5.

20. (canceled)

21. (canceled)

22. (canceled)

23. (canceled)

24. A method of enhancing an immune response, the method comprising administering a Toll-like Receptor (TLR) agonist and an agent that increases a TIM-3 activity to a subject in need of an enhanced immune response.

25. The method of claim 24, further comprising administering an antigen to which an immune response is to be generated with said TLR agonist and said agent that increases a TIM-3 activity.

26. The method of claim 24, wherein the TIM-3 activity is increased in antigen presenting cells (APCs) in the central nervous system.

27. (canceled)

28. (canceled)

29. The method of claim 24, wherein the agent is a TIM-3 ligand.

30. The method claim 29, wherein the TIM-3 ligand comprises a galectin-9 polypeptide.

31. (canceled)

32. (canceled)

33. (canceled)

34. The method of claim 24 wherein said subject has a tumor, and wherein said method treats said tumor.

35. The method of claim 34, wherein the tumor is a central nervous system tumor.

36. (canceled)

37. A vaccine composition comprising an agent that increases TIM-3 activity and a TLR ligand.

38. (canceled)

39. The vaccine composition of claim 37 further comprising an antigen to which an immune response is to be generated, wherein said antigen is not the agent that increases TIM-3 activity.

40. The vaccine composition according to claim 37, wherein the agent is an antibody, or antigen-binding fragment thereof, or a polypeptide.

41. The vaccine composition according to claim 40, wherein the antibody or antibody fragment binds TIM-3 and increases TIM-3 signaling.

42. The vaccine composition according to claim 37, wherein the agent is a TIM-3 ligand.

43. (canceled)

44. (canceled)

45. (canceled)

46. (canceled)

47. (canceled)

48. (canceled)

49. (canceled)

50. A method of vaccinating an animal against an antigen, the method comprising administering to the animal an agent that increases TIM-3 activity, and a TLR ligand.

51. The method of claim 50 further comprising administering said antigen to said animal, wherein said antigen is not the agent that increases TIM-3 activity.

52. (canceled)

53. (canceled)

54. (canceled)

55. (canceled)

56. (canceled)

57. (canceled)

58. (canceled)

59. (canceled)

60. (canceled)

61. (canceled)
